SEQUENCE LISTING (39 sequences)

SEQ ID NO: 1 - DNA, 2665 bp, Human
gtcgactggaggcccagctgcccgtcatcagagtgacaggtcttatgacagcctgattggtgactcgggctgggtgtggattctcaccccaggcctctgcctgctttctcagaccctcatctgtcacccccacgctgaacccagctgccacccccagaagcccatcagactgcccccagcacacggaatggatttctgagaaagaagccgaaacagaaggcccgtgggagtcagcatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatgaggctgactggatgtccagagctggccaggccactgggccctgagccagagacaaggtcacctgggctgtgatgtgaagacacctgcagcctttggtctcctggatgggcctttgagcctgatgtttacagtgtctgtgtgtgtgtgtgcatatgtgtgtgtgtgcatatgcatgtgtgtgtgtgtgtgtgtcttaggtgcgcagtggcatgtccacgtgtgtgtgtgattgcacgtgcctgtgggcctgggataatgcccatggtactccatgcattcacctgccctgtgcatgtctggactcacggagctcacccatgtgcacaagtgtgcacagtaaacgtgtttgtggtcaacagatgacaacagccgtcctccctcctagggtcttgtgttgcaagttggtccacagcatctccggggctttgtgggatcagggcattgcctgtgactgaggcggagcccagccctccagcgtctgcctccaggagctgcaagaagtccatattgttccttatcacctgccaacaggaagcgaaaggggatggagtgagcccatggtgacctcgggaatggcaattttttgggcggcccctggacgaaggtctgaatcccgactctgataccttctggctgtgctacctgagccaagtcgcctcccctctctgggctagagtttccttatccagacagtggggaaggcatgacacacctgggggaaattggcgatgtcacccgtgtacggtacgcagcccagagcagaccctcaataaacgtcagcttccttcaaaaaaaaaaaaaaaaatctaga

SEQ ID NO: 2 - protein, 538 aa, Human
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 3 - protein, 70 aa, Human
LMTNAFISIIDDLSKYDVQVRAAVSSMCREAGLWSEWSQPIYVGNDEHKPLREWFVIVIMATICFILLIL

SEQ ID NO: 4 - DNA, 25 bp, Artificial Sequence
  Description of Artificial Sequence PCR Primer
gagtccgaggagaaagctgatctca

SEQ ID NO: 5 - DNA, 24 bp, Artificial Sequence
  Description of Artificial Sequence PCR Primer
gaaagatgaccgggtcactccatt

SEQ ID NO: 6 - DNA, 29 bp, Artificial Sequence
  Description of Artificial Sequence Labelled HybridizationOligonucleotide
actcgagctatgagctgcaggtgcgggca

SEQ ID NO: 7 - DNA, 29 bp, Artificial Sequence
  Description of Artificial Sequence NN14-1b (MU-1) Labelled Hybridization Oligonucleotide
actcgagctatgagctgcaggtgcgggca

SEQ ID NO: 8 - protein, 5 aa, Artificial Sequence
  Description of Artificial Sequence Motif Characteristic of theHematopoietin Receptor Family
WSXWS

SEQ ID NO: 9 - DNA, 2628 bp, Mouse
gtcgacgcggcggtaccagctgtctgcccacttctcctgtggtgtgcctcacggtcacttgcttgtctgaccgcaagtctgcccatccctggggcagccaactggcctcagcccgtgccccaggcgtgccctgtctctgtctggctgccccagccctactgtcttcctctgtgtaggctctgcccagatgcccggctggtcctcagcctcaggactatctcagcagtgactcccctgattctggacttgcacctgactgaactcctgcccacctcaaaccttcacctcccaccaccaccactccgagtcccgctgtgactcccacgcccaggagaccacccaagtgccccagcctaaagaatggctttctgagaaagaccctgaaggagtaggtctgggacacagcatgccccggggcccactggctgccttactcctgctgattctccatggagcttggagctgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggaccctcacatgctgctgctcctggctgtcttgatcattgtcctggttttcatgggtctgaagatccacctgccttggaggctatggaaaaagatatgggcaccagtgcccacccctgagagtttcttccagcccctgtacagggagcacagcgggaacttcaagaaatgggttaatacccctttcacggcctccagcatagagttggtgccacagagttccacaacaacatcagccttacatctgtcattgtatccagccaaggagaagaagttcccggggctgccgggtctggaagagcaactggagtgtgatggaatgtctgagcctggtcactggtgcataatccccttggcagctggccaagcggtctcagcctacagtgaggagagagaccggccatatggtctggtgtccattgacacagtgactgtgggagatgcagagggcctgtgtgtctggccctgtagctgtgaggatgatggctatccagccatgaacctggatgctggccgagagtctggccctaattcagaggatctgctcttggtcacagaccctgcttttctgtcttgcggctgtgtctcaggtagtggtctcaggcttggaggctccccaggcagcctactggacaggttgaggctgtcatttgcaaaggaaggggactggacagcagacccaacctggagaactgggtccccaggagggggctctgagagtgaagcaggttccccccctggtctggacatggacacatttgacagtggctttgcaggttcagactgtggcagccccgtggagactgatgaaggaccccctcgaagctatctccgccagtgggtggtcaggacccctccacctgtggacagtggagcccagagcagctagcatataataaccagctatagtgagaagaggcctctgagcctggcatttacagtgtgaacatgtaggggtgtgtgtgtgtgtgtgtgtgtgtgtgtgtgtgtgtgtgtgtgtgtgtgtgtgtgtgtgtcttgggttgtgtgttagcacatccatgttgggatttggtctgttgctatgtattgtaatgctaaattctctacccaaagttctaggcctacgagtgaattctcatgtttacaaacttgctgtgtaaaccttgttccttaatttaataccattggttaaataaaattggctgcaaccaattactggagggattagaggtagggggcttttgagttacctgtttggagatggagaaggagagaggagagaccaagaggagaaggaggaaggagaggagaggagaggagaggagaggagaggagaggagaggagaggagaggagaggagaggctgccgtgaggggagagggaccatgagcctgtggccaggagaaacagcaagtatctggggtacactggtgaggaggtggccaggccagcagttagaagagtagattaggggtgacctccagtatttgtcaaagccaattaaaataacaaaaaaaaaaaaaaagcggccgctctaga

SEQ ID NO: 10 - protein, 529 aa, Mouse
MPRGPVAALLLLILHGAWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSS

SEQ ID NO: 11 - DNA, 20 bp, Artificial Sequence
  Description of Artificial Sequence PCR Primer
agcatcaagccggctccccc

SEQ ID NO: 12 - DNA, 20 bp, Artificial Sequence
  Description of Artificial Sequence PCR Primer
ctccattcactccaggtccc

SEQ ID NO: 13 - DNA, 20 bp, Artificial Sequence
  Description of Artificial Sequence PCR Primer
ttgaacgtgactgrggcctt

SEQ ID NO: 14 - DNA, 20 bp, Artificial Sequence
  Description of Artificial Sequence Murine MU-1 cDNA Internal Oligonucleotide
tgaatgaagtgcctggctga

SEQ ID NO: 15 - DNA, 40 bp, Artificial Sequence
  Description of Artificial Sequence 5′ PCR Primer
cacaaagcttcagtatgagctgcagtacaggaaccgggga

SEQ ID NO: 16 - DNA, 40 bp, Artificial Sequence
  Description of Artificial Sequence 3′ PCR primer
cacaggatccctttaactcctctgactgggtctgaaagat

SEQ ID NO: 17 - protein, 224 aa, Unknown Organism
  Unknown Organism (1)..(224): Description of Unknown Organism Second polypeptide comprising anFc region
HTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 617 bp, Human
gctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 19 - protein, 162 aa, Human
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 20 - protein, 7 aa, Human
EDDGYPA

SEQ ID NO: 21 - protein, 16 aa, Human
MPLLLLLLLLPSPLHP

SEQ ID NO: 22 - DNA, 786 bp, Human
atgaaattcttagtcaacgttgcccttgtttttatggtcgtgtacatttcttacatctatgccggcagcggacaccaccatcatcaccacggtagcggcgactataaagacgatgacgataagggttccggatgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaactaatga

SEQ ID NO: 23 - protein, 260 aa, Human
MKFLVNVALVFMVVYISYIYAGSGHHHHHHGSGDYKDDDDKGSGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWN

SEQ ID NO: 24 - DNA, 1426 bp, Human
gcggccgcaccaccatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaacggctccggctctagagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgaattc

SEQ ID NO: 25 - protein, 467 aa, Human
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNGSGSRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1499 bp, Human
gcggccgcaccaccatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaacggctccggctctagagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatcaggaatggcatcaatgacaggaggtcaacaaatgggttctggatctcatcatcatcatcatcattctggaggttgagaattc

SEQ ID NO: 27 - protein, 492 aa, Human
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNGSGSRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGMASMTGGQQMGSGSHHHHHHSGG

SEQ ID NO: 28 - DNA, 1426 bp, Human
gcggccgcaccaccatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaacggctccggctctagagacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgaattc

SEQ ID NO: 29 - protein, 467 aa, Human
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNGSGSRDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 741 bp, Human
atgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaacaaaaccgaaacctcccaggttgctccggcataatga

SEQ ID NO: 31 - protein, 245 aa, Human
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNKTETSQVAPA

SEQ ID NO: 32 - DNA, 1413 bp, Human
atgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaacgatgacgatgacaagggctccggcgacaaaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 33 - protein, 470 aa, Human
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNDDDDKGSGDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1754 bp, Mouse
atgccccggggcccagtggctgccttactcctgctgattctccatggagcttggagctgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggacggctccggctctagagagccccgcggaccgacaatcaagccctgtcctccatgcaaatgcccaggtaagtcactagaccagagctccactcccgggagaatggtaagtgctataaacatccctgcactagaggataagccatgtacagatccatttccatctctcctcatcagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggctttcgcatgcgccgtcaacaacaaagacctcccagcgcccatcgagagaaccatctcaaaacccaaaggtgagagctgcagcctgactgcatgggggctgggatgggcataaggataaaggtctgtgtggacagccttctgcttcagccatgacctttgtgtatgtttctaccctcacagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctcagcacccacaaaactctcaggtccaaagagacacccacactcatctccatgcttcccttgtataaataaagcacccagcaatgcctgggaccatgtaataggaattc

SEQ ID NO: 35 - protein, 240 aa, Mouse
MPRGPVAALLLLILHGAWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWDGSGSR

SEQ ID NO: 36 - DNA, 795 bp, Mouse
ctgcaggtcgacaccaccatgccccggggcccagtggctgccttactcctgctgattctccatggagcttggagctgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggacggcagcggacaccaccatcatcaccacggtagcggcgactataaagacgatgacgataagtagtgagaattc

SEQ ID NO: 37 - protein, 255 aa, Mouse
MPRGPVAALLLLILHGAWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWDGSGHHHHHHGSGDYKDDDDK

SEQ ID NO: 38 - DNA, 792 bp, Mouse
atgaaattcttagtcaacgttgcccttgtttttatggtcgtgtacatttcttacatctatgccggcagcggacaccaccatcatcaccacggtagcggcgactataaagacgatgacgataagggttccggatgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggactagtgagaattc

SEQ ID NO: 39 - protein, 260 aa, Mouse
MKFLVNVALVFMVVYISYIYAGSGHHHHHHGSGDYKDDDDKGSGCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWD

(19) United States
(12) Patent Application Publication
(10) Pub. No.: US 2008/0241098 A1
(43) Pub. Date: Oct. 2, 2008

(54) ANTAGONIZING INTERLEUKIN-21 RECEPTOR ACTIVITY

(75) Inventors: Deborah A. Young, Melrose, MA (US); Mary Collins, Natick, MA (US); Kyriaki Dunussi-Joannopoulos, Belmont, MA (US); Richard Michael O'Hara, Quincy, MA (US); Marion T. Kasaian, Charlestown, MA (US); Matthew J. Whitters, Hudson, MA (US)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 11/947,425

(22) Filed: Nov. 29, 2007

Related U.S. Application Data
(62) Division of application No. 11/197,488, filed on Aug. 5, 2005, now abandoned.
(60) Provisional application No. 60/599,086, filed on Aug. 5, 2004.
(60) Provisional application No. 60/639,176, filed on Dec. 23, 2004.

(51) Int. Cl. A61K 38/20; A61K 39/395; A61K 39/44; C07K 16/46; C12N 15/63; C12N 5/06; C12P 21/00; A61P 41/00

(52) U.S. Cl. 424/85.2; 424/158.1; 424/139.1; 424/178.1; 530/391.7; 435/320.1; 435/325; 435/69.7

(57) ABSTRACT

Methods and compositions for inhibiting interleukin-21 (IL-21)/IL-21 receptor (MU-1) activity using antagonists of IL-21 or IL-21 receptor (“IL-21R” or “MU-1”), are disclosed. IL-21/IL-21R antagonists can be used to induce immune suppression in vivo, e.g., for treating, ameliorating or preventing autoimmune or inflammatory disorders, including, e.g., inflammatory bowel disease (IBD), rheumatoid arthritis (RA), transplant/graft rejection, psoriasis, asthma, fibrosis, and systemic lupus erythematosus (SLE).

This application is a divisional of U.S. application Ser. No. 11/197,488, filed Aug. 5, 2005, which claims the benefit of U.S. Provisional Application No. 60/599,086, filed Aug. 5, 2004, and U.S. Provisional Application No. 60/639,176, filed Dec. 23, 2004, all of which are hereby incorporated by reference herein in their entireties.

### BACKGROUND OF THE INVENTION

1. Field of the Invention

The present invention relates to methods and compositions for antagonizing, reducing, and/or inhibiting interleukin-21 (IL-21)/IL-21 receptor (MU-1) activity using IL-21 receptor antagonists. The methods and compositions disclosed herein are useful as immunotherapeutic agents.

2. Related Background Art

Human IL-21 is a cytokine that shows sequence homology to IL-2, IL-4 and IL-15 (Parrish-Novak et al. (2000) Nature 408:57-63). Despite low sequence homology among interleukin cytokines, cytokines share a common fold into a “four-helix-bundle” structure that is representative of the family. Most cytokines bind either class I or class II cytokine receptors. Class II cytokine receptors include the receptors for IL-10 and the interferons, whereas class I cytokine receptors include the receptors for IL-2 through IL-7, IL-9, IL-11, IL-12, IL-13, and IL-15, as well as hematopoietic growth factors, leptin, and growth hormone (Cosman (1993) Cytokine 5:95-106).

Human IL-21 receptor (IL-21R) is a class I cytokine receptor that is expressed in lymphoid tissues, in particular by NK, B, and T cells (Parrish-Novak et al. (2000) supra). The nucleotide and amino acid sequences encoding human interleukin-21 (IL-21) and its receptor (IL-21R) are described in WO 00/53761; WO 01/85792; Parrish-Novak et al. (2000) supra; and Ozaki et al. (2000) Proc. Natl. Acad. Sci. U.S.A. 97:11439-44. IL-21R has the highest sequence homology to IL-2 receptor β chain and IL-4 receptor α chain (Ozaki et al. (2000) supra). Upon ligand binding, IL-21R associates with the common gamma cytokine receptor chain (γc) that is shared by receptors for IL-2, IL-3, IL-4, IL-7, IL-9, IL-13 and IL-15 (Ozaki et al. (2000) supra; Asao et al. (2001) J. Immunol. 167:1-5). The widespread lymphoid distribution of IL-21R suggests that IL-21 may play a role in immune regulation. Indeed, in vitro studies have shown that IL-21 significantly modulates the function of B cells, CD4⁺ and CD8⁺ T cells, and NK cells (Parrish-Novak et al. (2000) supra; Kasaian et al. (2002) Immunity. 16:559-69). Nevertheless, evidence supporting a regulatory effect of IL-21 in vivo is limited.

### SUMMARY OF THE INVENTION

Methods and compositions for interfering with the activity of and/or an interaction between interleukin-21 (IL-21) and an IL-21 receptor (also referred to herein as “IL-21R” or “MU-1”), e.g., using antagonists of IL-21 or IL-21R, are disclosed (also referred to herein as an “IL-21/IL-21R antagonist” or “antagonist” or “IL-21/IL-21R pathway antagonist”).

For example, Applicants have shown that reducing IL-21R activity by using an IL-21 antagonist, e.g., a fusion protein that includes the extracellular domain of the IL-21R fused to an Fc immunoglobulin region, ameliorates inflammatory symptoms in several different animal models reasonably predictive of inflammatory and/or autoimmune disorders, such as inflammatory bowel disease (IBD), rheumatoid arthritis (RA), transplant/graft rejection, graft vs. host disease, asthma, systemic lupus erythematosus (SLE) (including a form of glomerulonephritis), and psoriasis (Examples 7-14). Expression of IL-21R mRNA is upregulated in the paws of collagen-induced arthritis (CIA) mice (Example 8). Furthermore, a mouse deficient in IL-21R showed a reduction of symptoms in an asthma model (Example 12). Accordingly, antagonists of IL-21/IL-21R activity can be used to induce immune suppression in vivo, e.g., for treating or preventing inflammatory or autoimmune disorders. These antagonists can also be used to treat or prevent an immune cell-associated disorder, e.g., a disorder associated with aberrant activity of one or more of mature T cells (e.g., mature CD8+ or mature CD4+ T cells), mature NK cells, B cells, macrophages, and megakaryocytes.

Accordingly, in one aspect, the invention features a method of treating (e.g., curing, suppressing, delaying), ameliorating (e.g., lessening, alleviating, reducing, decreasing) and/or preventing (e.g., preventing the onset of, or preventing recurrence or relapse of) an inflammatory or an autoimmune disorder in a subject. The method includes: administering to the subject an IL-21/IL-21R antagonist, e.g., in an amount sufficient to treat, ameliorate, or prevent the disorder or in an amount sufficient to inhibit or reduce immune cell activity and/or cell number.

The IL-21/IL-21R antagonist can be administered to the subject alone, or in combinations of IL-21/IL-21R antagonists, or in combination with other therapeutic modalities as described herein. Preferably, the subject is a mammal, e.g., a human, suffering from or at risk for an inflammatory or an autoimmune disorder. For example, the method can be used to treat or prevent, in a subject, an inflammatory or an autoimmune disorder. Examples of such a disorder include, but are not limited to: transplant/graft rejection; diabetes mellitus (e.g., type I); multiple sclerosis; an arthritic disorder (e.g., rheumatoid arthritis (RA), juvenile rheumatoid arthritis, osteoarthritis, psoriatic arthritis, or ankylosing spondylitis (preferably, RA)); myasthenia gravis; vasculitis; systemic lupus erythematosus (SLE); glomerulonephritis; autoimmune thyroiditis; a skin inflammatory disorder (e.g., dermatitis (including atopic dermatitis and eczematous dermatitis), scleroderma, or psoriasis); lupus erythematosus; a fibrosis or fibrotic disorder (e.g., pulmonary fibrosis or liver fibrosis); a respiratory disorder (e.g., asthma or COPD); an atopic disorder (e.g., including allergy); or an intestinal inflammatory disorder (e.g., an IBD, e.g., Crohn's disease or ulcerative colitis).

Treatment of a disorder chosen from lupus erythematosus, a skin inflammatory disorder (e.g., psoriasis), an intestinal inflammatory disorder (e.g., IBD, Crohn's disease, ulcerative colitis), transplant/graft rejection, asthma, an atopic disorder, or rheumatoid arthritis, using the IL-21 or IL-21R antagonists of the present invention is preferred.

In one embodiment, the IL-21/IL-21R antagonist interacts with, e.g., binds to, IL-21 or IL-21R, preferably, mammalian, e.g., human IL-21 or IL-21R (referred to herein as an “IL-21 antagonist” and “IL-21R antagonist,” respectively), and reduces or inhibits one or more IL-21 and/or IL-21R activities. Preferred antagonists bind to IL-21 or IL-21R with high affinity, e.g., with an affinity constant of at least about 10⁷ M⁻¹, preferably about 10⁸ M⁻¹, and more preferably, about 10⁹ M⁻¹ to 10¹⁰ M⁻¹ or stronger.

For example, an IL-21/IL-21R antagonist can reduce and/or inhibit IL-21R activity by neutralizing IL-21. In one embodiment, the antagonist can be a fusion protein that includes a fragment of an IL-21R fused to a non-IL-21R fragment, e.g., an immunoglobulin Fc region. In other embodiments, the antagonist is an anti-IL-21R or anti-IL-21 antibody or an antigen-binding fragment thereof, a soluble form of the IL-21 receptor, a peptide or a small molecule inhibitor.

In one embodiment, the IL-21/IL-21R antagonist is an anti-IL-21R or anti-IL-21 antibody, or an antigen-binding fragment thereof; e.g., the antibody is a monoclonal or single specificity antibody that binds to IL-21, e.g., human IL-21, or an IL-21 receptor, e.g., human IL-21 receptor polypeptide, or an antigen-binding fragment thereof (e.g., an Fab, F(ab′), Fv or a single chain Fv fragment). Preferably, the antibody is a human, humanized, chimeric, or in vitro-generated antibody to human IL-21 or human IL-21 receptor polypeptide. Preferably, the antibody is a neutralizing antibody.

In other embodiments, the IL-21/IL-21R antagonist includes full length, or a fragment of an IL-21 polypeptide, e.g., an inhibitory IL-21 receptor-binding domain of an IL-21 polypeptide, e.g., a human IL-21 polypeptide (e.g., a human IL-21 polypeptide as described herein having an amino acid sequence shown as SEQ ID NO:19) or a sequence at least 85%, 90%, 95%, 98% or more identical thereto; or encoded by a corresponding nucleotide sequence shown as SEQ ID NO:18 or a sequence at least 85%, 90%, 95%, 98% or more identical thereto. Alternatively, the antagonist includes full length (e.g., from about amino acids 1-538 or 20-538 of SEQ ID NO:2; or from about amino acids 1-529 or 20-529 of SEQ ID NO:10), or a fragment of an IL-21 receptor polypeptide, e.g., an IL-21-binding domain of an IL-21 receptor polypeptide, e.g., a soluble fragment of an IL-21R (e.g., a fragment of an IL-21R comprising the extracellular domain of murine or human IL-21R; e.g., from about amino acids 1-235, 1-236, 20-235, 20-236 of SEQ ID NO:2 (human), or from about amino acids 1-236, 20-236 of SEQ ID NO:10 (murine), or encoded by the corresponding nucleotides of SEQ ID NO:1 or 9, or a sequence at least 85%, 90%, 95%, 98% or more identical thereto.

In one embodiment, the antagonist is a fusion protein comprising the aforesaid IL-21 or IL-21 receptor polypeptides or fragments thereof and, e.g., fused to a second moiety, e.g., a polypeptide (e.g., an immunoglobulin chain, a GST, Lex-A or MBP polypeptide sequence). In a preferred embodiment, the fusion protein includes at least a fragment of an IL-21R polypeptide that is capable of binding IL-21, e.g., a soluble fragment of an IL-21R (e.g., a fragment of an IL-21R comprising the extracellular domain of murine or human IL-21R, e.g., from about amino acids 1-235, 1-236, 20-235, 20-236 of SEQ ID NO:2 (human), or from about amino acids 1-236, 20-236 of SEQ ID NO:10 (murine), or encoded by the corresponding nucleotides of SEQ ID NO:1 or 9, or a sequence at least 85%, 90%, 95%, 98% or more identical thereto) and, e.g., fused to, a second moiety, e.g., a polypeptide (e.g., an immunoglobulin chain, an Fc fragment, a heavy chain constant regions of the various isotypes, including: IgG1, IgG2, IgG3, IgG4, IgM, IgA1, IgA2, IgD, and IgE). For example, the fusion protein can include the extracellular domain of human IL-21R, e.g., from about amino acids 1-235, 1-236, 20-235, 20-236 of SEQ ID NO:2, and, e.g., fused to, a human immunoglobulin Fc chain (e.g., human IgG, e.g., human IgG1, e.g., a naturally occurring human IgG1 or a mutated form of human IgG1). In one embodiment, the human Fc sequence has been mutated at one or more amino acids, e.g., mutated at residues 254 and 257 of SEQ ID NO:28, from the naturally occurring sequence to reduce Fc receptor binding. In other embodiments, the fusion protein can include the extracellular domain of murine IL-21R, e.g., from about amino acids 1-236, 20-235 of SEQ ID NO:10 (murine), and, e.g., fused to, a murine immunoglobulin Fc chain (e.g., murine IgG, e.g., murine IgG2a or a mutated form of murine IgG2a).

The fusion proteins may additionally include a linker sequence joining the first moiety, e.g., an IL-21R fragment, to the second moiety, e.g., the immunoglobulin fragment. In other embodiments, additional amino acid sequences can be added to the N— or C-terminus of the fusion protein to facilitate expression, steric flexibility, detection, and/or isolation or purification.

Examples of antagonistic fusion proteins that can be used in the methods of the invention are shown in FIGS. 7-15. In one embodiment, the fusion protein includes an amino acid sequence chosen from, e.g., SEQ ID NO:23, SEQ ID NO:25, SEQ ID NO:27, SEQ ID NO:29, SEQ ID NO:31, SEQ ID NO:33, SEQ ID NO:35, SEQ ID NO:37, or SEQ ID NO:39, or a sequence at least 85%, 90%, 95%, 98% or more identical thereto. In other embodiments, the fusion protein includes an amino acid sequence encoded by a nucleotide sequence chosen from, e.g., SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38, or a sequence at least 85%, 90%, 95%, 98% or more identical thereto. Preferred fusion proteins have the amino acid sequence shown as SEQ ID NO:25 or SEQ ID NO:29 (FIGS. 8A-8C and 10A-10C, respectively), or a sequence at least 85%, 90%, 95%, 98% or more identical thereto. In other embodiments, the fusion protein includes an amino acid sequence encoded by a nucleotide sequence chosen from, e.g., SEQ ID NO:24 or SEQ ID NO:28 (FIGS. 8A-8C and 10A-10C, respectively), or a sequence at least 85%, 90%, 95%, 98% or more identical thereto. Most preferably, the fusion protein has the amino acid sequence shown as SEQ ID NO:29 or has an amino acid sequence encoded by a nucleotide sequence shown as SEQ ID NO:28 (FIG. 10A-10C).

The IL-21/IL-21R antagonists described herein, e.g., the fusion protein described herein, can be derivatized or linked to another functional molecule, e.g., another peptide or protein (e.g., an Fab′ fragment). For example, the fusion protein or an antibody, or antigen-binding portion, can be functionally linked (e.g., by chemical coupling, genetic fusion, noncovalent association or otherwise) to one or more other molecular entities, such as an antibody (e.g., a bispecific or a multispecific antibody), toxins, radioisotopes, cytotoxic or cytostatic agents, among others.

In one embodiment, the IL-21/IL-21R antagonists described herein, e.g., the pharmaceutical compositions thereof, are administered in combination therapy, i.e., combined with other agents, e.g., therapeutic agents, which are useful for treating inflammatory or autoimmune disorders, e.g., a disorder chosen from one or more of: an arthritic disorder (including RA, juvenile rheumatoid arthritis, osteoarthritis, psoriatic arthritis, or ankylosing spondylitis); SLE; glomerulonephritis; a skin inflammatory disorder (e.g., psoriasis); a respiratory disorder (e.g., asthma, COPD); an atopic disorder; a fibrotic disorder (e.g., pulmonary fibrosis or liver fibrosis); an intestinal inflammatory disorder (e.g., an IBD, e.g., Crohn's disease or ulcerative colitis); or transplant/graft rejection. For example, the combination therapy can include one or more IL-21/IL-21R antagonists, e.g., an anti-IL-21 or anti-IL-21R antibody or an antigen-binding fragment thereof; an IL-21R fusion protein; a soluble IL-21R receptor; a peptide inhibitor or a small molecule inhibitor) coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described herein.

Examples of preferred additional therapeutic agents that can be coadministered and/or coformulated with one or more IL-21/IL-21R antagonists, include, but are not limited to, one or more of: TNF antagonists (e.g., chimeric, humanized, human or in vitro-generated antibodies, or antigen-binding fragments thereof, that bind to TNF; soluble fragments of a TNF receptor, e.g., p55 or p75 human TNF receptor or derivatives thereof, e.g., 75 kdTNFR-IgG (75 kDa TNF receptor-IgG fusion protein, ENBREL™), p55 kDa TNF receptor-IgG fusion protein; TNF enzyme antagonists, e.g., TNFα converting enzyme (TACE) inhibitors); antagonists of IL-6, IL-12, IL-15, IL-17, IL-18, IL-22; T cell and B cell depleting agents (e.g., anti-CD4 or anti-CD22 antibodies); small molecule inhibitors, e.g., methotrexate and leflunomide; sirolimus (rapamycin) and analogs thereof, e.g., CCI-779; Cox-2 and cPLA2 inhibitors; NSAIDs; p38 inhibitors, TPL-2, Mk-2 and NFib inhibitors; RAGE or soluble RAGE; P-selectin or PSGL-1 inhibitors (e.g., small molecule inhibitors, antibodies thereto, e.g., antibodies to P-selectin); estrogen receptor beta (ERB) agonists or ERB-NFκb antagonists. Most preferred additional therapeutic agents that can be coadministered and/or coformulated with one or more IL-21/IL-21R antagonists include one or more of: a soluble fragment of a TNF receptor, e.g., p55 or p75 human TNF receptor or derivatives thereof, e.g., 75 kdTNFR-IgG (75 kDa TNF receptor-IgG fusion protein, ENBREL™); methotrexate, leflunomide, or sirolimus (rapamycin) or an analog thereof, e.g., CCI-779.

In another aspect, a method for decreasing immune cell activity (e.g., the activity of one or more of: a mature T cell (mature CD8⁺, CD4⁺, lymph node T cell, memory T cell), mature NK cell, B cell, antigen presenting cell (APC), e.g., a dendritic cell, macrophage or megakaryocyte, or a population of cells, e.g., a mixed or a substantially purified immune cell population, is provided. The method includes contacting the immune cell with an IL-21/IL-21R antagonist, e.g., an antagonist as described herein, in an amount sufficient to decrease immune cell activity.

In another aspect, the invention features a fusion protein that includes at least a fragment of an IL-21R polypeptide, which is capable of binding an IL-21 polypeptide, e.g., a soluble fragment of an IL-21R (e.g., a fragment of an IL-21R comprising the extracellular domain of murine or human IL-21R; e.g., from about amino acids 1-235, 1-236, 20-235, 20-236 of SEQ ID NO:2 (human), or from about amino acids 1-236, 20-236 of SEQ ID NO:10 (murine), or encoded by the corresponding nucleotides of SEQ ID NO:1 or SEQ ID NO:9, or a sequence at least 85%, 90%, 95%, 98% or more identical thereto) and, e.g., fused to, a second moiety, e.g., a polypeptide (e.g., an immunoglobulin chain, an Fc fragment, a heavy chain constant regions of the various isotypes, including: IgG1, IgG2, IgG3, IgG4, IgM, IgA1, IgA2, IgD, and IgE). For example, the fusion protein can include the extracellular domain of human IL-21R, e.g., from about amino acids 1-235, 1-236, 20-235, 20-236 of SEQ ID NO:2, and, e.g., fused to, a human immunoglobulin Fc chain (e.g., human IgG, e.g., human IgG1 or a mutated form of human IgG1). In one embodiment, the human Fc sequence has been mutated at one or more amino acids, e.g., mutated at residues 254 and 257 of SEQ ID NO:28, from the wild type sequence to reduce Fc receptor binding. In other embodiments, the fusion protein can include the extracellular domain of murine IL-21R, e.g., from about amino acids 1-236, 20-236 of SEQ ID NO:10 (murine), and, e.g., fused to, a murine immunoglobulin Fc chain (e.g., murine IgG, e.g., murine IgG2a or a mutated form of murine IgG2a). The fusion proteins may additionally include a linker sequence joining the IL-21R fragment to the second moiety. In other embodiments, additional amino acid sequences can be added to the N— or C-terminus of the fusion protein to facilitate expression, detection and/or isolation or purification.

The invention also features nucleic acid sequences that encode the fusion proteins described herein.

In another aspect, the invention features host cells and vectors containing the nucleic acids of the invention. Preferably, the host cell is a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. For example, nucleic acids encoding the fusion proteins described herein can be expressed in a transgenic animal. In one embodiment, the nucleic acids are placed under the control of a tissue-specific promoter (e.g., a mammary-specific promoter) and the antibody is produced in the transgenic animal. For example, the fusion protein is secreted into the milk of the transgenic animal, such as a transgenic cow, pig, horse, sheep, goat, or rodent.

In another aspect, the invention provides a process for producing a fusion protein, e.g., a fusion protein as described herein. The process comprises: (a) growing a culture of the host cell of the present invention in a suitable culture medium and (b) purifying the fusion protein from the culture. Proteins produced according to these methods are also provided.

In another aspect, the invention provides compositions, e.g., pharmaceutical compositions, which include a pharmaceutically acceptable carrier and at least one of IL-21/IL-21R antagonist as described herein (e.g., a fusion protein described herein). In one embodiment, the compositions, e.g., pharmaceutical compositions, comprise a combination of two or more IL-21/IL-21R antagonists. Combinations of the IL-21/IL-21R antagonists and a drug, e.g., a therapeutic agent (e.g., one or more cytokine and growth factor inhibitors, immunosuppressants, anti-inflammatory agents, metabolic inhibitors, enzyme inhibitors, and/or cytotoxic or cytostatic agents, as described herein) or an antigen, e.g., an antigenic peptide and/or an antigen-presenting cell, are also within the scope of the invention.

In one embodiment, the pharmaceutical composition includes an IL-21/IL-21R antagonist and at least one additional therapeutic agent, in a pharmaceutically acceptable carrier. Examples of preferred additional therapeutic agents that can be coformulated in a composition, e.g., a pharmaceutical composition, with one or more IL-21/IL-21R antagonists, include, but are not limited to, one or more of: TNF antagonists (e.g., chimeric, humanized, human or in vitro-generated antibodies, or antigen-binding fragments thereof, that bind to TNF; soluble fragments of a TNF receptor, e.g., p55 or p75 human TNF receptor or derivatives thereof, e.g., 75 kdTNFR-IgG (75 kDa TNF receptor-IgG fusion protein, ENBREL™), p55 kDa TNF receptor-IgG fusion protein; TNF enzyme antagonists, e.g., TNFα converting enzyme (TACE) inhibitors); antagonists of IL-6, IL-12, IL-15, IL-17, IL-18, IL-22; T cell and B cell depleting agents (e.g., anti-CD4 or anti-CD22 antibodies); small molecule inhibitors, e.g., methotrexate and leflunomide; sirolimus (rapamycin) and analogs thereof, e.g., CCI-779; Cox-2 and cPLA2 inhibitors; NSAIDs; p38 inhibitors, TPL-2, Mk-2 and NFκb inhibitors; RAGE or soluble RAGE; P-selectin or PSGL-1 inhibitors (e.g., small molecule inhibitors, antibodies thereto, e.g., antibodies to P-selectin); estrogen receptor beta (ERB) agonists or ERB-NFκb antagonists. Most preferred additional therapeutic agents that can be coadministered and/or coformulated with one or more IL-21/IL-21R antagonists include one or more of: a soluble fragment of a TNF receptor, e.g., p55 or p75 human TNF receptor or derivatives thereof, e.g., 75 kdTNFR-IgG (75 kDa TNF receptor-IgG fusion protein, ENBREL™); methotrexate, leflunomide, or sirolimus (rapamycin) or an analog thereof, e.g., CCI-779.

In another aspect, the invention features methods to treat, ameliorate, or prevent an atopic disorder in a subject, e.g., a mammal, e.g., a human. The method includes: administering to the subject an IL-21/IL-21R antagonist, e.g., in an amount sufficient to treat, ameliorate, or prevent the disorder or in an amount sufficient to inhibit or reduce immune cell activity and/or cell number. In one embodiment, the atopic disorder is allergic asthma. In another embodiment, the atopic disorder is atopic dermatitis, urticaria, eczema, allergic rhinitis, or allergic enterogastritis. In one embodiment, the IL-21/IL-21R antagonist can be administered in combination with another therapeutic agent, e.g., a cytokine inhibitor, an immunosuppressant, an anti-inflammatory agent, an enzyme inhibitor, a leukotriene antagonist, a bronchodilator, a beta 2-adrenoceptor agonist, an antimuscarinic, or a mast cell stabilizer. Examples of preferred therapeutic agents that can be administered in conjunction with an IL-21/IL-21R antagonist to treat, ameliorate, or prevent an atopic disorder include, e.g., TNF antagonists, IL-6 antagonists, IL-12 antagonists, IL-15 antagonists, IL-17 antagonists, IL-18 antagonists, IL-22 antagonists, T cell-depleting agents, B cell-depleting agents, methotrexate, leflunomide, sirolimus (rapamycin) or analogs thereof, Cox-2 inhibitors, cPLA2 inhibitors, NSAIDs, and p38 inhibitors.

In another aspect, the invention features methods to treat, ameliorate, or prevent an autoimmune disorder in a subject. The method includes: administering to the subject an IL-21/IL-21R antagonist, e.g., in an amount sufficient to treat, ameliorate, or prevent the disorder or an in amount sufficient to inhibit or reduce immune cell activity and/or cell number. In one embodiment, the autoimmune disorder is lupus, e.g., SLE. In one embodiment, the IL-21/IL-21R antagonist can be administered in combination with another therapeutic agent, e.g., a cytokine inhibitor, a growth factor inhibitor, an immunosuppressant, an anti-inflammatory agent, a metabolic inhibitor, an enzyme inhibitor, a cytotoxic agent, or a cytostatic agent. Examples of preferred therapeutic agents that can be administered in conjunction with an IL-21/IL-21R antagonist to treat, ameliorate, or prevent an autoimmune disorder include, e.g., TNF antagonists, IL-6 antagonists, IL-12 antagonists, IL-15 antagonists, IL-17 antagonists, IL-18 antagonists, IL-22 antagonists, T cell-depleting agents, B cell-depleting agents, chloroquine, hydroxychloroquine, methotrexate, leflunomide, sirolimus (rapamycin) or analogs thereof, Cox-2 inhibitors, cPLA2 inhibitors, NSAIDs, and p38 inhibitors.

In another aspect, the invention features methods to treat, ameliorate, or prevent a fibrotic disorder in a subject. The method includes: administering to the subject an IL-21/IL-21R antagonist, e.g., in an amount sufficient to treat, ameliorate, or prevent the disorder or an in amount sufficient to inhibit or reduce immune cell activity and/or cell number. For example, the subject may have or be at risk for fibrosis of an internal organ (e.g., liver fibrosis, renal fibrosis, or pulmonary fibrosis), a dermal fibrosing disorder, or a fibrotic condition of the eye.

In another aspect, the invention features methods of transplanting or grafting organs, tissues, or cells to a subject. The method includes administering to the subject an IL-21/IL-21R antagonist, e.g., before, during, or after the transplantation or grafting. The organs and tissues transplanted/grafted can include, but are not limited to, e.g., heart, kidney, liver, lung, pancreas, bone marrow, cartilage, cornea, neuronal tissue, and cells thereof. In one embodiment, the IL-21/IL-21R antagonist is administered in combination with another therapeutic agent, e.g., a cytokine inhibitor, a growth factor inhibitor, an immunosuppressant, an anti-inflammatory agent, a metabolic inhibitor, an enzyme inhibitor, a cytotoxic agent, and a cytostatic agent. Examples of preferred therapeutic agents that can be administered in conjunction with IL-21/IL-21R antagonists include, e.g., rapamycin, cyclosporine, anti-CTLA-4 antibodies, anti-CD40 antibodies, anti-CD40L antibodies, and anti-CD154 antibodies.

In another aspect, the invention features a method of evaluating and treating a transplant/graft recipient for symptoms of transplant/graft rejection or a disorder associated with transplant/graft rejection, e.g., fibrosis or graft-versus-host-disease (GVHD). The method includes identifying a subject with symptoms of transplant/graft rejection and administering an IL-21/IL-21R antagonist, e.g., in an amount sufficient to treat or ameliorate the symptoms of transplant rejection. Symptoms of transplant/graft rejection include, e.g., inflammation, decreased organ function, abnormal biopsy, and fibrosis. In another embodiment, the invention provides a method of preventing (e.g., reducing the risk of) transplant/graft rejection or a disorder associated with transplant/graft rejection by administering an IL-21/IL-21R antagonist.

In another aspect, the invention features methods to treat, ameliorate, or prevent transplant/graft rejection or a disorder associated with transplant/graft rejection in a subject. The method features administering to the subject an IL-21/IL-21R antagonist in an amount sufficient to treat, ameliorate, or prevent (e.g., reduce the risk of) the rejection or in an amount sufficient to inhibit or reduce immune cell activity and/or cell number. The organs or tissues transplanted can include, e.g., heart, kidney, liver, lung, pancreas, and bone marrow. In one embodiment, the IL-21/IL-21R antagonist can be administered in combination with another therapeutic agent, e.g., a cytokine inhibitor, a growth factor inhibitor, an immunosuppressant, an anti-inflammatory agent, a metabolic inhibitor, an enzyme inhibitor, a cytotoxic agent, or a cytostatic agent. Examples of preferred therapeutic agents that can be administered in conjunction with IL-21/IL-21R antagonists to treat, ameliorate, or prevent transplant/graft rejection include, e.g., rapamycin, cyclosporine, anti-CTLA-4 antibodies, anti-CD40 antibodies, anti-CD40L antibodies, and anti-CD154 antibodies.

The following sets of terms are used interchangeably herein: “MU-1” and “IL-21R,” and peptides, polypeptides, and proteins.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 depicts the full-length cDNA sequence of murine IL-21R/MU-1. The nucleotide sequence corresponds to nucleotides 1-2628 of SEQ ID NO:9.

FIGS. 2A-2B depict the amino acid sequences of murine and human IL-21R/ MU-1. FIG. 2A depicts the amino acid sequence of murine IL-21R/MU-1 (corresponding to the amino acids 1-529 of SEQ ID NO:10). There is a predicted leader sequence at amino acids 1-19 (predicted by SPScan) with score of 10.1 (bold-face type). There is a predicted transmembrane domain at amino acids 237-253 of SEQ ID NO:10 (underlined). Predicted signaling motifs include the following regions: Box 1: amino acids 265-274 and Box 2: amino acids 310-324 (bold and underlined); six tyrosines are located at amino acid positions 281, 319, 361, 368, 397, and 510, of SEQ ID NO:10. The WSXWS motif (SEQ ID NO:8) is located at amino acid residue 214 to amino acid residue 218 (in large, bold-face type). Potential STAT docking sites include, amino acids 393-398 and amino acids 510-513 of SEQ ID NO:10. FIG. 2B depicts the amino acid sequence of human MU-1 (corresponding to SEQ ID NO:2). The location of the predicted signal sequence (about amino acids 1-19 of SEQ ID NO:2); WSXWS motif (about amino acids 213-217 of SEQ ID NO:2); and transmembrane domain (about amino acids 236-252, 236-253, 236-254, of SEQ ID NO:2 (underlined)) are indicated. Potential JAK binding sites, signaling motifs and STAT docking sites are also indicated. The approximate location of these sites is boxed.

FIG. 3 depicts the GAP comparison of human and murine MU-1 cDNA sequences (corresponding to nucleic acids 1-2665 of SEQ ID NO:1 and nucleic acids 1-2628 of SEQ ID NO:9, respectively). HuMU-1=human MU-1, murMU-1=murine MU-1. Gap Parameters: Gap Weight=50, Average Match=10.000, Length Weight=3, Average Mismatch=0.000, Percent Identity=66.116.

FIG. 4 depicts a GAP comparison of the human MU-1 protein (corresponding to amino acids of SEQ ID NO:2) and the murine MU-1 protein (corresponding to amino acids of SEQ ID NO:10). The alignment was generated by BLOSUM62 amino acid substitution matrix (Henikoff and Henikoff (1992) Proc. Natl. Acad. Sci. U.S.A. 89:10915-19). Gap parameters=Gap Weight: 8, Average Match=2.9 12, Length Weight=2, Average Mismatch=−2.003; Percent Identity=65.267.

FIG. 5 depicts a multiple sequence alignment of the amino acids of human MU-1 (corresponding to SEQ ID NO:2), murine MU-1 (corresponding to SEQ ID NO: 10), and human IL2 beta chain (GENBANK® Accession No. M26062). Leader and transmembrane domains are underlined. Conserved cytokine receptor module motifs are indicated by bold-face type. Potential signaling regions are indicated by underlining and bold-face type.

FIG. 6 depicts signaling through MU-1. MU-1 phosphorylates STAT 5 in Clone E7 EPO-MU-1 chimera. Under the conditions specified in Example 3, signaling through MU-1 results in the phosphorylation of STAT 5 at all time-points tested. Treatment of controls or the chimeric BAF-3 cells with IL-3 resulted in phosphorylation of STAT 3, but not STAT 1 or 5.

FIGS. 7A-7B depict an alignment of the nucleotide and amino acid sequences of human IL-21R monomer (corresponding to amino acids 20-235 of SEQ ID NO:2) fused at the amino terminal to honey bee leader sequence and His6 tags (amino acids 1-44 of SEQ ID NO:23). The nucleotide and amino acid sequences are shown as SEQ ID NO:22 and SEQ ID NO:23, respectively.

FIGS. 8A-8C depict an alignment of the nucleotide and amino acid sequences of human IL-21R extracellular domain (corresponding to amino acids 1-235 of SEQ ID NO:2) fused at the C-terminus via a linker (corresponding to amino acids 236-243 of SEQ ID NO:25) to human immunoglobulin G1 (IgG1) Fc sequence (corresponding to amino acids 244-467 of SEQ ID NO:25). The nucleotide and amino acid sequences are shown as SEQ ID NO:24 and SEQ ID NO:25, respectively.

FIGS. 9A-9C depict an alignment of the nucleotide and amino acid sequences of human IL-21R extracellular domain (corresponding to amino acids 1-235 of SEQ ID NO:2) fused at the C-terminus via a linker (corresponding to amino acids 236-243 of SEQ ID NO:27) to human immunoglobulin G1 (IgG1) Fc sequence (corresponding to amino acids 244-467 of SEQ ID NO:27), and His₆ sequence tag (corresponding to amino acids 468-492 of SEQ ID NO:27). The nucleotide and amino acid sequences are shown as SEQ ID NO:26 and SEQ ID NO:27, respectively.

FIGS. 10A-10C depict an alignment of the nucleotide and amino acid sequences of human IL-21R extracellular domain (corresponding to amino acids 1-235 of SEQ ID NO:2) fused at the C-terminus via a linker (corresponding to amino acids 236-243 of SEQ ID NO:29) to human immunoglobulin G1 (IgG1) Fc mutated sequence (corresponding to amino acids 244-467 of SEQ ID NO:29). The human Fc sequence has been mutated at residues 254 and 257 from the wild-type sequence to reduce Fc receptor binding. The nucleotide and amino acid sequences are shown as SEQ ID NO:28 and SEQ ID NO:29, respectively.

FIGS. 11A-11B depict an alignment of the nucleotide and amino acid sequences of human IL-21R extracellular domain (corresponding to amino acids 1-235 of SEQ ID NO:2) fused at the C-terminus to a rhodopsin sequence tag. The nucleotide and amino acid sequences are shown as SEQ ID NO:30 and SEQ ID NO:31, respectively.

FIGS. 12A-12C depict an alignment of the nucleotide and amino acid sequences of human IL-21R extracellular domain (corresponding to amino acids 1-235 of SEQ ID NO:2) fused at the C-terminus to an EK cleavage site and mutated IgG1 Fc region (corresponding to amino acids 236-470 of SEQ ID NO:33). The nucleotide and amino acid sequences are shown as SEQ ID NO:32 and SEQ ID NO:33, respectively.

FIGS. 13A-13B depict an alignment of the nucleotide and amino acid sequences of murine IL-21R extracellular domain fused at the C-terminus to mouse immunoglobulin G2a (IgG2a). The nucleotide (genomic) and amino acid sequences are shown as SEQ ID NO:34 and SEQ ID NO:35, respectively.

FIGS. 14A-14B depict an alignment of the nucleotide and amino acid sequences of murine IL-21R extracellular domain fused at the C-terminus to Flag and His₆ sequence tags. The nucleotide (genomic) and amino acid sequences are shown as SEQ ID NO:36 and SEQ ID NO:37, respectively.

FIGS. 15A-15B depict an alignment of the nucleotide and amino acid sequences of (honey bee leader) murine IL21R extracellular domain fused at the C-terminus to Flag and His₆ sequence tags. The nucleotide (genomic) and amino acid sequences are shown as SEQ ID NO:38 and SEQ ID NO:39, respectively.

FIG. 16 is a timetable summarizing the prophylactic, therapeutic and semi-therapeutic treatment schedules for the experiments using collagen-induced arthritis (CIA) mouse models.

FIG. 17 is a graph depicting the effects of MuIL-21RFc (200 μg/mouse 3×/week) on a semi-therapeutic CIA mouse as a function of days post-treatment. Mouse Ig (200 μg/mouse 3×/week) was used as a control.

FIGS. 18A-18B are photographs showing increased expression of IL-21R mRNA in arthritic paws of mice with CIA (panel A) compared to negative controls (panel B).

FIGS. 19 and 20 depict linear graphs showing a marked reduction in the clinical score of IBD-like symptoms in rats treated with muIL-21RFc and mEnbrel, compared to the IgG control. FIG. 19, left side panel, is a photograph showing in situ hybridization of MU-1 mRNA in the lymphocytes and lymph nodes of the normal human intestine.

FIG. 21 is a table summarizing a reduction in histological scoring of disease severity in a rat IBD model after administration of MuIL-21RFc.

FIG. 22 is a linear graph showing the percentage of graft survival relative to days post-adoptive transfer in mice injected with retrovirally transduced T cells expressing IL-21, muIL-21RFc or control (GFP).

FIG. 23 is a linear graph showing an improvement of clinical scores in psoriatic lesions in a CD45RB^{hi} adoptive transfer model after administration of MuIL-21RFc. FIG. 23, left hand side, shows photographs of mice before and after treatment with MuIL-21RFc.

FIG. 24 is a line graph depicting the levels of airway hyperresponsiveness (AHR) of ovalbumin (OVA)-sensitized mice challenged with either phosphate buffered saline (PBS) or OVA. Mice were administered sequentially increasing doses of methacholine. The Penh (enhanced pause) change is an indicator of AHR.

FIGS. 25A-25D are bar graphs depicting numbers of cells in bronchoalveolar lavage fluid (BALF) of OVA-sensitized mice challenged with either PBS or OVA. FIG. 25A depicts total BALF cell numbers. FIG. 25B depicts numbers of eosinophils in BALF. FIG. 25C depicts numbers of lymphocytes in BALF. FIG. 25D depicts numbers of neutrophils in BALF. Unfilled bars indicate PBS-challenged WT mice; filled bars indicate OVA-challenged WT mice; gray bars indicate PBS-challenged IL-21R −/− mice; hatched bars indicate OVA-challenged IL-21R −/− mice. *indicates p<0.05 as determined by Mann-Whitney U test.

FIGS. 26 and 27 are graphs depicting levels of cytokines in BALF of OVA-sensitized mice challenged with OVA. FIG. 26 depicts levels of TNFα and IL-5. FIG. 27 depicts levels of IL-13. Unfilled bars indicate PBS-challenged WT mice; filled bars indicate OVA-challenged WT mice; gray bars indicate PBS-challenged IL-21R −/− mice; hatched bars indicate OVA-challenged IL-21R −/− mice. *indicates p<0.05 as determined by Mann-Whitney U test.

FIGS. 28A-28B are bar graphs depicting levels of serum IgE in OVA-sensitized mice challenged with OVA or PBS. FIG. 28A depicts levels of total serum IgE. FIG. 28B depicts levels of anti-OVA specific IgE. Unfilled bars indicate PBS-challenged WT mice; filled bars indicate OVA-challenged WT mice; gray bars indicate PBS-challenged IL-21R −/− mice; hatched bars indicate OVA-challenged IL-21R −/− mice. *indicates p<0.05 as determined by Mann-Whitney U test.

FIGS. 29A-29D are graphs depicting the levels of circulating dsDNA autoantibodies in MRL-Fas^{lpr} mice following treatment with MuIL-21RFc or control. FIG. 29A depicts levels of IgG1. FIG. 29B depicts levels of IgG2a. FIG. 29C depicts levels of IgG2b. FIG. 29D depicts levels of IgG3. *indicates p<0.05 as determined by Mann-Whitney U test.

FIGS. 30A-30D are graphs depicting circulating total IgG in MRL-Fas^{lpr} mice following treatment with MuIL-21RFc or control. FIG. 30A depicts levels of IgG1. FIG. 30B depicts levels of IgG2a. FIG. 30C depicts levels of IgG2b. FIG. 30D depicts levels of IgG3. *indicates p<0.05 as determined by Mann-Whitney U test.

FIG. 31 is a graph depicting levels of fluorescence in mouse kidney slices stained with goat anti-mouse IgG-FITC.

FIG. 32 is a schematic diagram depicting exemplary effects of IL-21 on immune responses.

FIG. 33 is a schematic diagram depicting exemplary strategies for inhibiting the IL-21/IL-21R pathway.

FIG. 34 is a schematic diagram depicting an exemplary soluble IL-21RFc receptor fusion protein.

FIG. 35 is a line graph depicting the mean psoriasis score of MuIL-21RFc-treated and control-treated groups of mice stimulated with E. tenella (“Etenella”).

FIG. 36 is a table summarizing a delay in onset and reduction of symptoms of psoriasis in E. tenella-stimulated mice treated with MuIL-21RFc compared to control-treated mice.

FIG. 37 is a line graph depicting a reduction in weight loss in E. tenella-stimulated mice treated with MuIL-21RFc compared to control treated mice. Weight index is defined as the ratio of weight measured to initial weight.

FIG. 38A is a line graph depicting a reduction in mean stool score in E. tenella-stimulated mice treated with MuIL-21RFc compared to control-treated mice.

FIG. 38B is a graph depicting stool scores of individual E. tenella-stimulated mice of each treatment group at day 77 post transfer.

FIG. 39 is a table summarizing the data depicted in FIG. 38A.

FIG. 40A is a graph depicting serum IFN-γ levels in E. tenella-stimulated mice treated with MuIL-21RFc compared to control-treated mice.

FIG. 40B is a graph depicting stool scores for E. tenella-stimulated mice treated with MuIL-21RFc compared to control-treated mice.

FIG. 41 is a line graph depicting ³H-thymidine incorporation into activated CD45RB^{hi} and CD45RB^{lo} cells following treatment with IL-21.

FIGS. 42A-B are bar graphs depicting an increase in secretion of cytokines by activated CD45RB^{hi} cells following IL-21 treatment.

FIG. 43 is a bar graph depicting a reduction in secretion of cytokines by activated CD45RB^{hi} cells following treatment with MuIL-21RFc.

FIG. 44A-B are bar (A) and scatter (B) graphs depicting that, in the GVHD model of SLE, IL-21R knockout mice engrafted with B6 bm12 spleen cells do not make anti-dsDNA autoantibodies (A) and IgG deposition is not observed in the kidneys of these mice (B).

### DETAILED DESCRIPTION OF THE INVENTION

Methods and compositions for inhibiting interleukin-21 (IL-21)/IL-21 receptor (MU-1) activity using antagonists of IL-21 or IL-21 receptor (“IL-21R” or “MU-1”), are disclosed. IL-21/IL-21R antagonists can be used to induce immune suppression in vivo, e.g., for treating or preventing inflammatory or autoimmune disorders (e.g., disorders associated with aberrant activity of one or more of mature T cells (mature CD8⁺, mature CD4⁺ T cells), mature NK cells, B cells, macrophages and megakaryocytes, including transplant/graft rejection, psoriasis and autoimmune disorders such as rheumatoid arthritis and IBD).

In one embodiment, Applicants have shown that a reduction of IL-21R activity by using a neutralizing fusion protein that includes the extracellular domain of the IL-21R fused to an Fc immunoglobulin region ameliorates inflammatory symptoms in collagen-induced arthritis (CIA) animal models (Example 7), as well as animal models for IBD (Examples 9 and 11), graft rejection (Example 10), psoriasis (Example 11), and lupus (Example 13). Expression of IL-21R mRNA is upregulated in the paws of CIA mice (Example 8). Mice deficient in IL-21R show a reduction in antigen-induced airway inflammation (Example 12). Accordingly, IL-21R binding agents that antagonize IL-21/IL-21R activity can be used to induce immune suppression in vivo, e.g., for treating or preventing inflammatory or autoimmune disorders (e.g., glomerulonephritis, transplant/graft rejection, psoriasis, atopic disorders, asthma, autoimmune disorders such as rheumatoid arthritis and SLE, and IBD (e.g., Crohn's disease, ulcerative colitis)).

In order that the present invention may be more readily understood, certain terms are first defined. Additional definitions are set forth throughout the detailed description.

The term “MU-1,” “MU-1 protein,” “interleukin-21 receptor” or “IL-21R,” as used herein, refers to a class I cytokine family receptor, also known as NILR (WO 01/85792; Parrish-Novak et al. (2000) Nature 408:57-63; Ozaki et al. (2000) Proc. Natl. Acad. Sci. U.S.A. 97:11439-444). MU-1 is homologous to the shared β chain of the IL-2 and IL-15 receptors, and IL-4α (Ozaki et al. (2000) supra). Upon ligand binding, IL-21R/MU-1 is capable of interacting with a common γ cytokine receptor chain (γc) (Asao et al. (2001) J. Immunol. 167:1-5), and inducing the phosphorylation of STAT1 and STAT3 (Asao et al. (2001) supra) or STAT5 (Ozaki et al. (2000) supra). MU-1 shows widespread lymphoid tissue distribution. The tern “MU-1” refers to a receptor (preferably of mammalian, e.g., murine or human origin) which is capable of interacting with, e.g., binding to, IL-21 (preferably of mammalian, e.g., murine or human IL-21) and having one of the following features: (i) an amino acid sequence of a naturally occurring mammalian MU-1 polypeptide IL-21R/MU-1 or a fragment thereof, e.g., an amino acid sequence shown as SEQ ID NO:2 (human) or SEQ ID NO:10 (murine) or a fragment thereof; (ii) an amino acid sequence substantially homologous to, e.g., at least 85%, 90%, 95%, 98%, or 99% homologous to, an amino acid sequence shown as SEQ ID NO:2 (human) or SEQ ID NO:10 (murine) or a fragment thereof; (iii) an amino acid sequence that is encoded by a naturally occurring mammalian IL-21/R/MU-1 nucleotide sequence or a fragment thereof (e.g., SEQ ID NO:1 (human) or SEQ ID NO:9 (murine) or a fragment thereof); (iv) an amino acid sequence encoded by a nucleotide sequence which is substantially homologous to, e.g., at least 85%, 90%, 95%, 98%, 99% homologous to, a nucleotide sequence shown as SEQ ID NO:1 (human) or SEQ ID NO:9 (murine) or a fragment thereof; (v) an amino acid sequence encoded by a nucleotide sequence degenerate to a naturally occurring IL-21R/MU-1 nucleotide sequence or a fragment thereof, e.g., SEQ ID NO:1 (human) or SEQ ID NO:9 (murine) or a fragment thereof; or (vi) a nucleotide sequence that hybridizes to one of the foregoing nucleotide sequences under stringent conditions, e.g., highly stringent conditions.

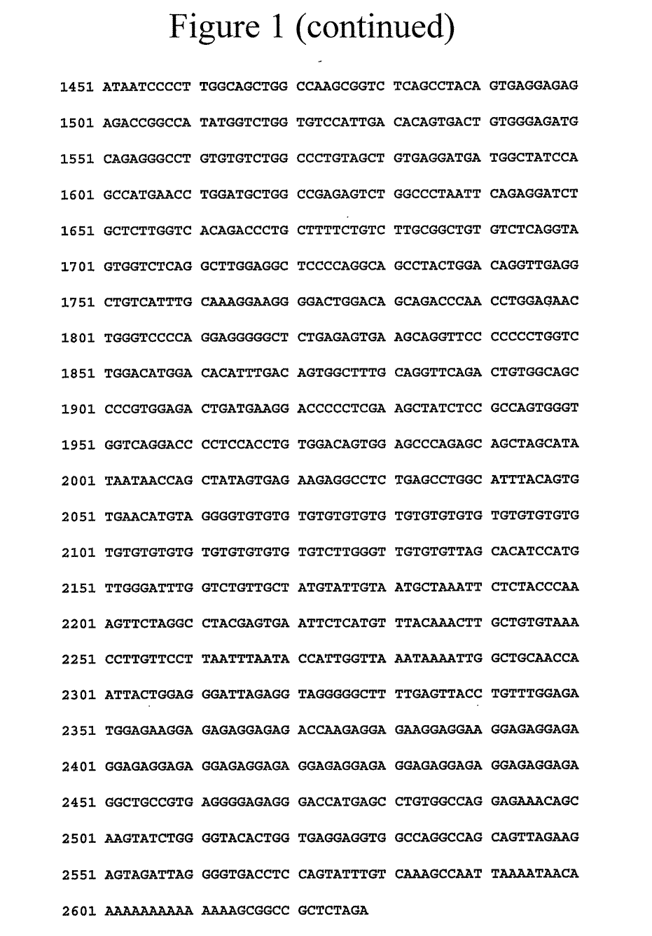

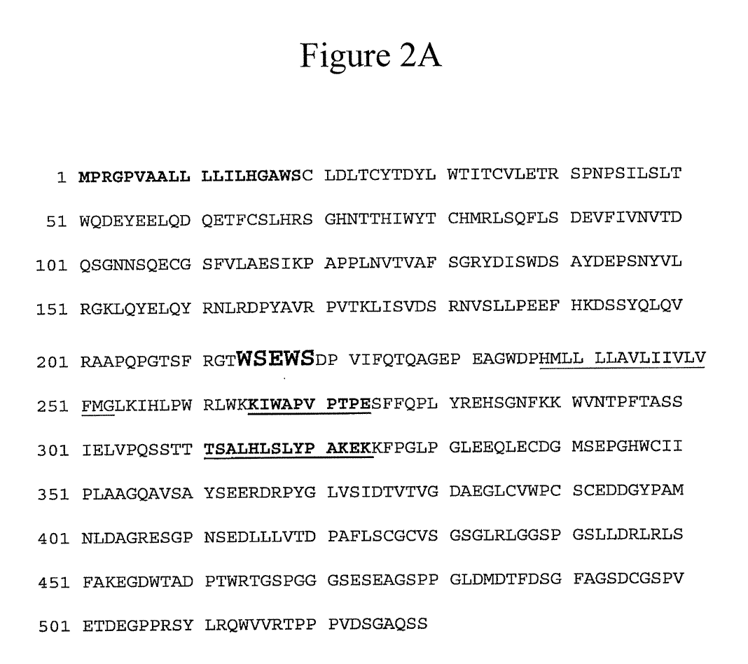

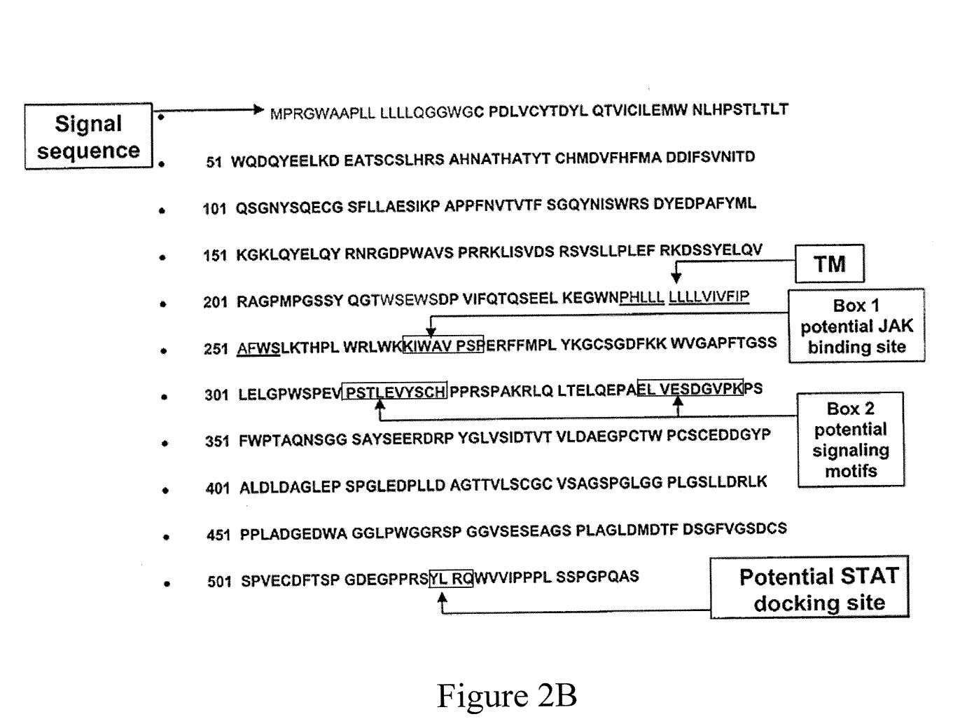

The IL-2/R/MU-1 is of mammalian, preferably, human origin. The nucleotide sequence and the predicted amino acid sequence of human IL-21R/MU-1 are shown in SEQ ID NO:1 and SEQ ID NO:2, respectively. Analysis of the human IL-21R/MU-1 amino acid sequence (SEQ ID NO:2; FIG. 2B) revealed the following structural features: a leader sequence (about amino acids 1-19 of SEQ ID NO:2 (FIG. 2B)); WSXWS motif (about amino acids 213-217 of SEQ ID NO:2); transmembrane domain (about amino acids 236-252 of SEQ ID NO:2 (FIG. 2B)); an extracellular domain from about amino acids 1-235 of SEQ ID NO:2; and an intracellular domain from about 253-538 of SEQ ID NO:2. The mature human IL-21R/MU-1 is believed to have the sequence of amino acids 20-538 of SEQ ID NO:2.

The IL-21R/MU-1 cDNA was deposited with the American Type Culture Collection on Mar. 10, 1998, as accession number ATCC 98687.

Any form of L-21R/MU-1 proteins of less than full length can be used in the methods and compositions of the present invention, provided that it retains the ability to bind to an IL-21 polypeptide. IL-21R/MU-1 proteins of less than full length, e.g., soluble IL-21R, can be produced by expressing a corresponding fragment of the polynucleotide encoding the full-length MU-1 protein in a host cell. These corresponding polynucleotide fragments are also part of the present invention. Modified polynucleotides as described above may be made by standard molecular biology techniques, including construction of appropriate desired deletion mutants, site-directed mutagenesis methods or by polymerase chain reaction using appropriate oligonucleotide primers.

As used herein, a “soluble IL-21R/MU-1 polypeptide” is an IL-21R/MU-1 polypeptide incapable of anchoring itself in a membrane. Such soluble polypeptides include, for example, MU-1 or IL-21R polypeptides that lack a sufficient portion of their membrane-spanning domain to anchor the polypeptide or are modified such that the membrane-spanning domain is nonfunctional, e.g., a soluble fragment of an IL-21R (e.g., a fragment of an IL-21R comprising the extracellular domain of murine or human IL-21R includes an amino acid sequence from about amino acids 1-235, 1-236, 20-235, 20-236 of SEQ ID NO:2 (human), or from about amino acids 1-236, 20-236 of SEQ ID NO:10 (murine). A soluble IL-21R/MU-1 polypeptide can additionally include, e.g., be fused to, a second moiety, e.g., a polypeptide (e.g., an immunoglobulin chain, a GST, Lex-A or MBP polypeptide sequence). For example, a fusion protein can include at least a fragment of an IL-21R polypeptide, which is capable of binding IL-21, e.g., a soluble fragment of an IL-21R (e.g., a fragment of an IL-21R comprising the extracellular domain of murine or human IL-21R; e.g., from about amino acids 1-235, 1-236, 20-235, 20-236 of SEQ ID NO:2 (human), or from about amino acids 1-236, 20-236 of SEQ ID NO:10 (murine), fused to a second moiety, e.g., a polypeptide (e.g., an immunoglobulin chain, an Fc fragment, a heavy chain constant region of the various isotypes, including: IgG1, IgG2, IgG3, IgG4, IgM, IgA1, IgA2, IgD, and IgE).

The term “interleukin-21” or “IL-21” refers to a cytokine showing sequence homology to IL-2, IL-4 and IL-15 (Parrish-Novak et al. (2000) supra). Despite low sequence homology among interleukin cytokines, cytokines share a common fold into a “four-helix-bundle” structure that is representative of the family. It is expressed primarily in activated CD4+ T cells, and has been reported to have effects on NK, B and T cells (Parrish-Novak et al. (2000) supra; Kasaian et al. (2002) supra). IL-21 binds to IL-21R (also referred to herein as MU-1 and NILR). Upon IL-21 binding, activation of IL-21R leads to STAT5 or STAT3 signaling (Ozaki et al. (2000) supra). The term “IL-21” or “IL-21 polypeptide” refers to a protein (preferably of mammalian, e.g., murine or human origin) which is capable of interacting with, e.g., binding to, IL-21R (preferably of mammalian, e.g., murine or human origin) and having one of the following features: (i) an amino acid sequence of a naturally occurring mammalian IL-21 or a fragment thereof, e.g., an amino acid sequence shown as SEQ ID NO:19 (human) or a fragment thereof; (ii) an amino acid sequence substantially homologous to, e.g., at least 85%, 90%, 95%, 98%, or 99% homologous to, an amino acid sequence shown as SEQ ID NO:19 (human) or a fragment thereof; (iii) an amino acid sequence which is encoded by a naturally occurring mammalian IL-21 nucleotide sequence or a fragment thereof (e.g., SEQ ID NO:18 (human) or a fragment thereof); (iv) an amino acid sequence encoded by a nucleotide sequence which is substantially homologous to, e.g., at least 85%, 90%, 95%, 98%, or 99% homologous to, a nucleotide sequence shown as SEQ ID NO:18 (human) or a fragment thereof; (v) an amino acid sequence encoded by a nucleotide sequence degenerate to a naturally occurring IL-21 nucleotide sequence or a fragment thereof, e.g., SEQ ID NO:19 (human) or a fragment thereof; or (vi) a nucleotide sequence that hybridizes to one of the foregoing nucleotide sequences under stringent conditions, e.g., highly stringent conditions.

The phrase “a biological activity of” a MU-1 or IL-21R polypeptide refers to one or more of the biological activities of the corresponding mature MU-1 protein, including, but not limited to, (1) interacting with, e.g., binding to, an IL-21 polypeptide (e.g., a human IL-21 polypeptide); (2) associating with signal transduction molecules, e.g., γc, JAK1; (3) stimulating phosphorylation and/or activation of stat proteins, e.g., STAT5 and/or STAT3; and/or (4) modulating, e.g., stimulating or decreasing, proliferation, differentiation, effector cell function, cytolytic activity, cytokine secretion, and/or survival of immune cells, e.g., T cells (CD8+, CD4+ T cells), NK cells, B cells, macrophages and megakaryocytes).

As used herein, an “IL-21/IL-21R antagonist” that is useful in the method of the invention refers to an agent which reduces, inhibits or otherwise diminishes one or more biological activities of an IL-21R/MU-1 polypeptide. In one preferred embodiment, the antagonist interacts with, e.g., binds to, an IL-21R/MU-1 polypeptide. In another preferred embodiment, the antagonist interacts with, e.g., binds to, an IL-21 polypeptide. Antagonism using an IL-21/IL-21R antagonist does not necessarily indicate a total elimination of the biological activity of the IL-21R/MU-1 polypeptide and/or the IL-21 polypeptide.

As used herein, a “therapeutically effective amount” of an IL-21/IL-21R antagonist refers to an amount of an agent which is effective, upon single or multiple dose administration to a subject, e.g., a human patient, at curing, reducing the severity of, ameliorating, or preventing one or more symptoms of a disorder, or in prolonging the survival of the subject beyond that expected in the absence of such treatment.

As used herein, “a prophylactically effective amount” of an IL-21/IL-21R antagonist refers to an amount of an IL-21/IL-21R antagonist which is effective, upon single or multiple dose administration to a subject, e.g., a human patient, in preventing or delaying the occurrence of the onset or recurrence of a disorder, e.g., a disorder as described herein.

The terms “induce,” “inhibit,” “potentiate,” “elevate,” “increase,” “decrease” or the like, e.g., which denote quantitative differences between two states, refer to at least statistically significant differences between the two states.

The term “in combination” in this context means that the agents are given substantially contemporaneously, either simultaneously or sequentially. If given sequentially, at the onset of administration of the second compound, the first of the two compounds is preferably still detectable at effective concentrations at the site of treatment or in the subject.

As used herein, a “fusion protein” refers to a protein containing two or more operably associated, e.g., linked, moieties, e.g., protein moieties. Preferably, the moieties are covalently associated. The moieties can be directly associated, or connected via a spacer or linker.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, e.g., Kabat et al. (1991) Sequences of Proteins of Immunological Interest, Fifth Edition, U.S. Department of Health and Human Services, NIH Publication No. 91-3242, and Chothia et al. (1987) J. Mol. Biol. 196:901-17, which are incorporated herein by reference). Each VH and VL is composed of three CDRs and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are interconnected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH-terminus. Full-length immunoglobulin “heavy chains” (about 50 kDa or 446 amino acids), are similarly encoded by a variable region gene (about 116 amino acids) and one of the other aforementioned constant region genes, e.g., gamma (encoding about 330 amino acids).

As used herein, “isotype” refers to the antibody class (e.g., IgM or IgG1) that is encoded by heavy chain constant region genes.

The term “antigen-binding fragment” of an antibody (or simply “antibody portion,” or “fragment”), as used herein, refers to one or more fragments of a full-length antibody that retain the ability to specifically bind to an antigen (e.g., CD3). Examples of binding fragments encompassed within the term “antigen-binding fragment” of an antibody include (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-26; and Huston et al. (1988) Proc. Natl. Acad Sci. U.S.A. 85:5879-83). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

Sequences similar or homologous (e.g., at least about 85% sequence identity) to the sequences disclosed herein are also part of this application. In some embodiments, the sequence identity can be about 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions) to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and nonhomologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J Mol. Biol. 48:444-53) algorithm which has been incorporated into the GAP program in the GCG software package (available at www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS, 4:11-17), which has been incorporated into the ALIGN program (version 2.0), using a PAM 120 weight residue table, a gap length penalty of 12 and a gap penalty of 4.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6×SSC at about 45° C., followed by one or more washes in 0.2×SSC, 0.1% SDS at 55° C. A further example of stringent hybridization conditions are hybridization in 6×SSC at about 45° C., followed by one or more washes in 0.2×SSC, 0.1% SDS at 60° C. Preferably, stringent hybridization conditions are hybridization in 6×SSC at about 45° C., followed by one or more washes in 0.2×SSC, 0.1% SDS at 65° C. Particularly preferred highly stringent conditions (and the conditions that should be used if the practitioner is uncertain about what conditions should be applied to determine if a molecule is within a hybridization limitation of the invention) are 0.5M sodium phosphate, 7% SDS at 65° C., followed by one or more washes at 0.2×SSC, 1% SDS at 65° C. The isolated polynucleotides of the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridizations, in situ hybridization and Northern hybridization, and are well known to those skilled in the art. Further disclosure regarding hybridization conditions and reactions is provided herein.

Hybridization reactions can be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| | | | Hybridization | |
|---|---|---|---|---|
| Stringency | Poly-nucleotide | Hybrid Length | Temperature and | Wash Temperature |
| Condition | Hybrid | (bp)¹ | Buffer² | and Buffer² |
| A | DNA:DNA | >50 | 65° C.; 1X SSC -or- | 65° C.; 0.3X SSC |
| | | | 42° C.; 1X SSC, | |
| | | | 50% formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X SSC |
| | | | 45° C.; 1X SSC, | |
| | | | 50% formamide | |
| D | DNA:RNA | <50 | T_{D}*; 1X SSC | T_{D}*; 1X SSC |
| E | RNA:RNA | >50 | 70° C.; 1X SSC -or- | 70° C.; 0.3X SSC |
| | | | 50° C.; 1X SSC, | |
| | | | 50% formamide | |
| F | RNA:RNA | <50 | T_{F}*; 1X SSC | T_{F}*; 1X SSC |
| G | DNA:DNA | >50 | 65° C.; 4X SSC -or- | 65° C.; 1X SSC |
| | | | 42° C.; 4X SSC, | |
| | | | 50% formamide | |
| H | DNA:DNA | <50 | T_{H}*; 4X SSC | T_{H}*; 4X SSC |
| I | DNA:RNA | >50 | 67° C.; 4X SSC -or- | 67° C.; 1X SSC |
| | | | 45° C.; 4X SSC, | |
| | | | 50% formamide | |
| J | DNA:RNA | <50 | T_{J}*; 4X SSC | T_{J}*; 4X SSC |
| K | RNA:RNA | >50 | 70° C.; 4X SSC -or- | 67° C.; 1X SSC |
| | | | 50° C.; 4X SSC, | |
| | | | 50% formamide | |
| L | RNA:RNA | <50 | T_{L}*; 2X SSC | T_{L}*; 2X SSC |
| M | DNA:DNA | >50 | 50° C.; 4X SSC -or- | 50° C.; 2X SSC |
| | | | 40° C.; 6X SSC, | |
| | | | 50% formamide | |
| N | DNA:DNA | <50 | T_{N}*; 6X SSC | T_{N}*; 6X SSC |
| O | DNA:RNA | >50 | 55° C.; 4X SSC -or- | 55° C.; 2X SSC |
| | | | 42° C.; 6X SSC, | |
| | | | 50% formamide | |
| P | DNA:RNA | <50 | T_{P}*; 6X SSC | T_{P}*; 6X SSC |
| Q | RNA:RNA | >50 | 60° C.; 4X SSC -or- | 60° C.; 2X SSC |
| | | | 45° C.; 6X SSC, | |
| | | | 50% formamide | |
| R | RNA:RNA | <50 | T_{R}*; 4X SSC | T_{R}*; 4X SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be lessthan 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1X SSC = 0.165 M).
Additional examples of stringency conditions for polynucleotide hybridization are provided in Sambrook et al., Molecular Cloning: A Laboratory Manual, Chs. 9 & 11, Cold Spring Harbor Laboratory Press, Cold Spring Harbor, NY (1989), and Ausubel et al., eds., Current Protocols in Molecular Biology, Sects. 2.10 & 6.3-6.4, John Wiley & Sons, Inc. (1995), herein incorporated by reference.

The isolated polynucleotides of the present invention may be used as hybridization probes and primers to identify and isolate DNA having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least 90% sequence identity (more preferably, at least 95% identity; most preferably, at least 99% identity) with the disclosed polynucleotides.

The isolated polynucleotides of the present invention may also be used as hybridization probes and primers to identify and isolate DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. Preferably, polynucleotide homologs have at least 50% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

The isolated polynucleotides of the present invention may also be used as hybridization probes and primers to identify cells and tissues that express the polypeptides of the present invention and the conditions under which they are expressed.

It is understood that the IL-21/IL-21R antagonists of the present invention may have additional conservative or nonessential amino acid substitutions, which do not have a substantial effect on their functions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “recombinant host cell” (or simply “host cell”), as used herein, is intended to refer to a cell into which a recombinant expression vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

### IL-21/IL-21R Antagonists

In one embodiment, an IL-21R/MU-1 polypeptide or active fragments thereof may be fused to a second moiety, e.g., an immunoglobulin or a fragment thereof (e.g., an Fc binding fragment thereof). For example, soluble forms of the IL-21R/MU-1 may be fused through “linker” sequences to the Fc portion of an immunoglobulin. Other fusions proteins, such as those with GST, Lex-A or MBP, may also be used.

The fusion proteins may additionally include a linker sequence joining the IL-21 or IL-21R fragment to the second moiety. For example, the fusion protein can include a peptide linker, e.g., a peptide linker of about 4 to 20, more preferably, 5 to 10, amino acids in length; in one embodiment, the peptide linker is 8 amino acids in length. Each of the amino acids in the peptide linker is selected from the group consisting of Gly, Ser, Asn, Thr and Ala; in one embodiment, the peptide linker includes a Gly-Ser element. In other embodiments, the fusion protein includes a peptide linker and the peptide linker includes a sequence having the formula (Ser-Gly-Gly-Gly-Gly) wherein y is 1, 2, 3, 4, 5, 6, 7, or 8.

In other embodiments, additional amino acid sequences can be added to the N— or C-terminus of the fusion protein to facilitate expression, detection and/or isolation or purification. For example, IL-21/IL-21R fusion protein may be linked to one or more additional moieties, e.g., GST, His₆ tag, or FLAG tag. For example, the fusion protein may additionally be linked to a GST fusion protein in which the fusion protein sequences are fused to the C-terminus of the GST (i.e., glutathione S-transferase) sequences. Such fusion proteins can facilitate the purification of the MU-1 fusion protein.

In another embodiment, the fusion protein includes a heterologous signal sequence (i.e., a polypeptide sequence that is not present in a polypeptide encoded by a MU-1 nucleic acid) at its N-terminus. For example, the native MU-1 signal sequence can be removed and replaced with a signal sequence from another protein. In certain host cells (e.g., mammalian host cells), expression and/or secretion of MU-1 can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Moreover, many expression vectors are commercially available that encode a fusion moiety (e.g., an Fe region of an immunoglobulin heavy chain). A MU-1-encoding nucleic acid can be cloned into such an expression vector such that the fusion moiety is linked in-frame to the immunoglobulin protein. In some embodiments, MU-1 fusion polypeptides exist as oligomers, such as dimers or trimers. The first polypeptide, and/or nucleic acids encoding the first polypeptide, can be constructed using methods known in the art.

In some embodiments, the MU-1 polypeptide moiety is provided as a variant MU-1 polypeptide having a mutation in the naturally occurring MU-1 sequence (wild type) that results in higher affinity (relative to the nonmutated sequence) binding of the MU-1 polypeptide to an IL-21.

In some embodiments, the MU-1 polypeptide moiety is provided as a variant MU-1 polypeptide having mutations in the naturally occurring MU-1 sequence (wild type) that results in a MU-1 sequence more resistant to proteolysis (relative to the nonmutated sequence). In some embodiments, the first polypeptide includes full-length MU-1 polypeptide. Alternatively, the first polypeptide comprises less than full-length MU-1 polypeptide.

A signal peptide that can be included in the fusion protein is MPLLLLLLLLPSPLHP (SEQ ID NO:21). If desired, one or more amino acids can additionally be inserted between the first polypeptide moiety comprising the MU-1 moiety and the second polypeptide moiety.

The second polypeptide is preferably soluble. In some embodiments, the second polypeptide enhances the half-life, (e.g., the serum half-life) of the linked polypeptide. In some embodiments, the second polypeptide includes a sequence that facilitates association of the fusion polypeptide with a second MU-1 polypeptide. In preferred embodiments, the second polypeptide includes at least a region of an immunoglobulin polypeptide. Immunoglobulin fusion polypeptides are known in the art and are described in, e.g., U.S. Pat. Nos. 5,516,964; 5,225,538; 5,428,130; 5,514,582; 5,714,147; and 5,455,165.

In some embodiments, the second polypeptide comprises a full-length immunoglobulin polypeptide. Alternatively, the second polypeptide comprises less than full-length immunoglobulin polypeptide, e.g., a heavy chain, light chain, Fab, Fab₂, Fv, or Fc. Preferably, the second polypeptide includes the heavy chain of an immunoglobulin polypeptide. More preferably, the second polypeptide includes the Fc region of an immunoglobulin polypeptide.

In another aspect of the invention, the second polypeptide has less effector function than the effector function of an Fc region of a wild-type immunoglobulin heavy chain. Fc effector function includes for example, Fc receptor binding, complement fixation and T cell depleting activity (see, e.g., U.S. Pat. No. 6,136,310). Methods for assaying T cell-depleting activity, Fc effector function, and antibody stability are known in the art. In one embodiment, the second polypeptide has low or no affinity for the Fc receptor. In an alternative embodiment, the second polypeptide has low or no affinity for complement protein C1q.

A preferred second polypeptide sequence includes the amino acid sequence of SEQ ID NO:17. This sequence includes an Fc region. Underlined amino acids are those that differ from the amino acid found in the corresponding position of the wild-type immunoglobulin sequence:

(SEQ ID NO: 17)

HTCPPCPAPEALGAPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPVPIEKTISKAKGQPREPQVYTLPPSREEMTKNQVSLTCLVKGFY
PSDIAVEWESNGQPENNYKTTPPVLDSDGSFFLYSKLTVDKSRWQ
QGNVFSCSVMHEALHNHYTQKSLSLSPGK

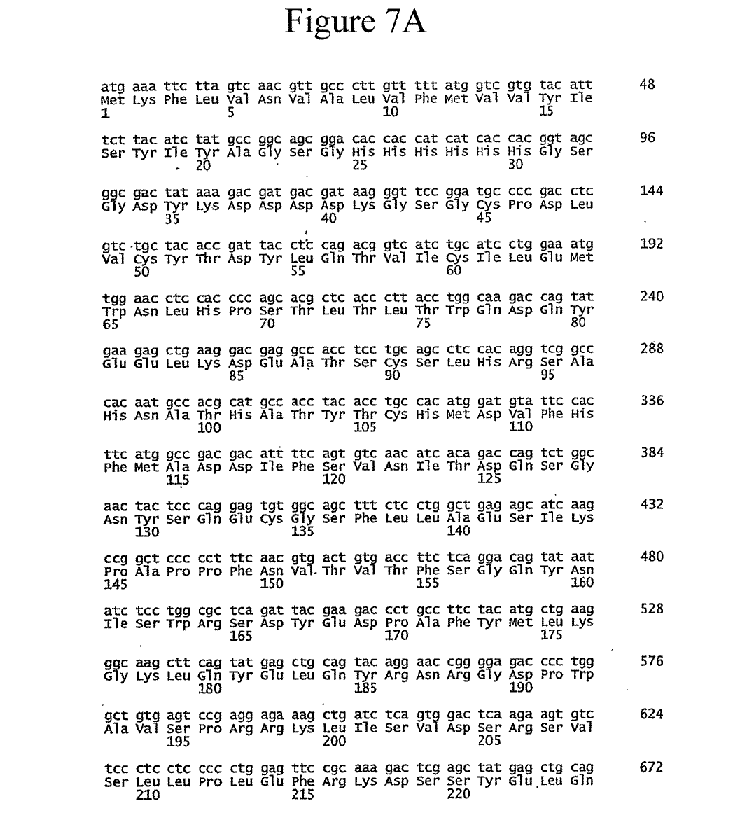

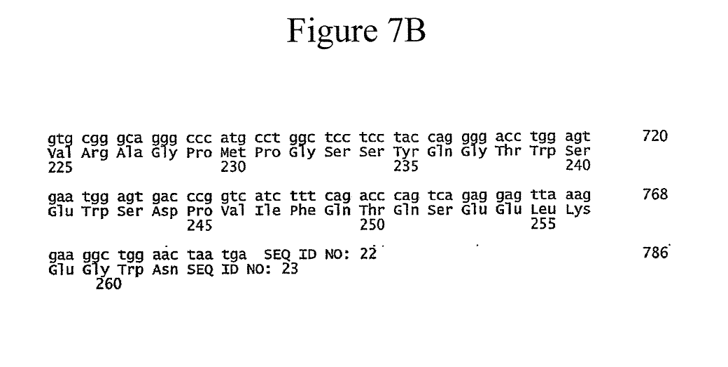

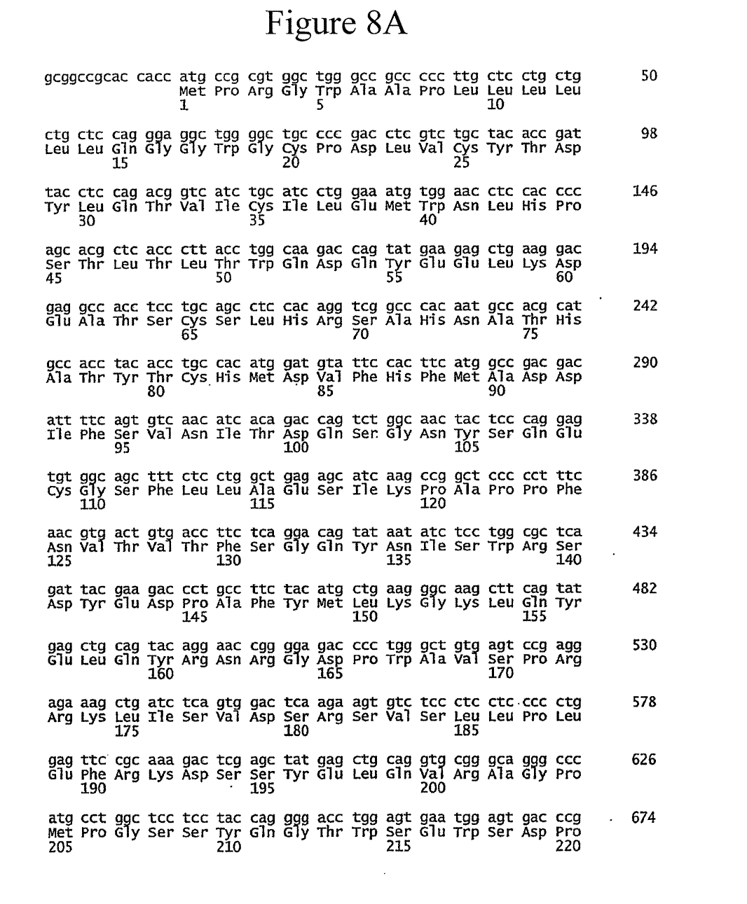

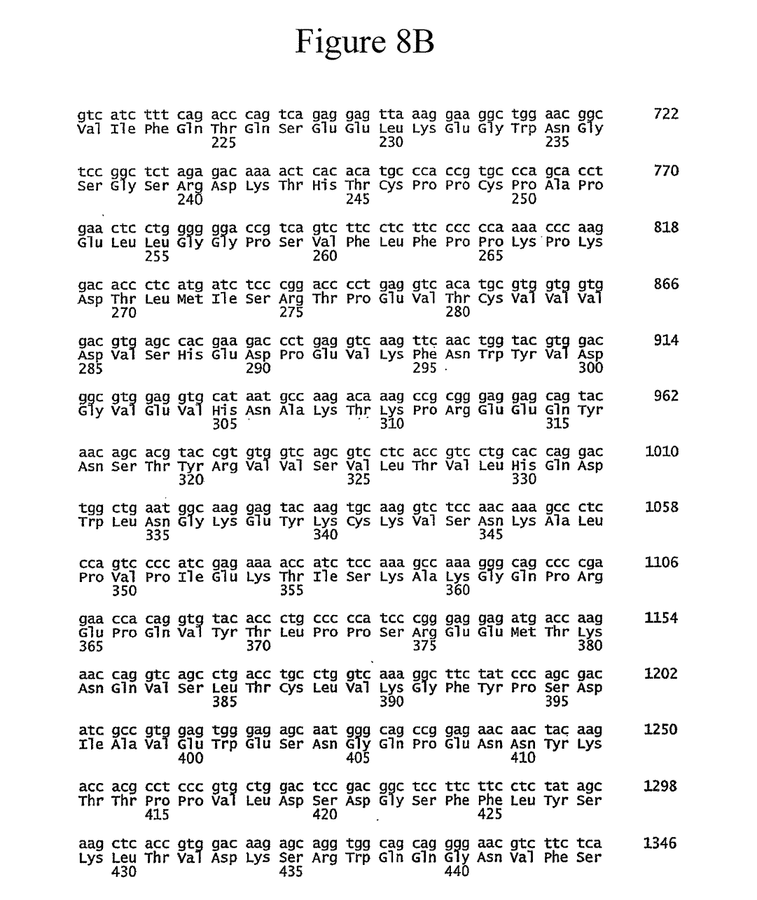

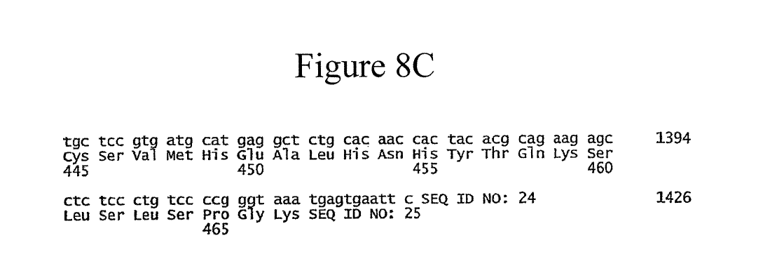

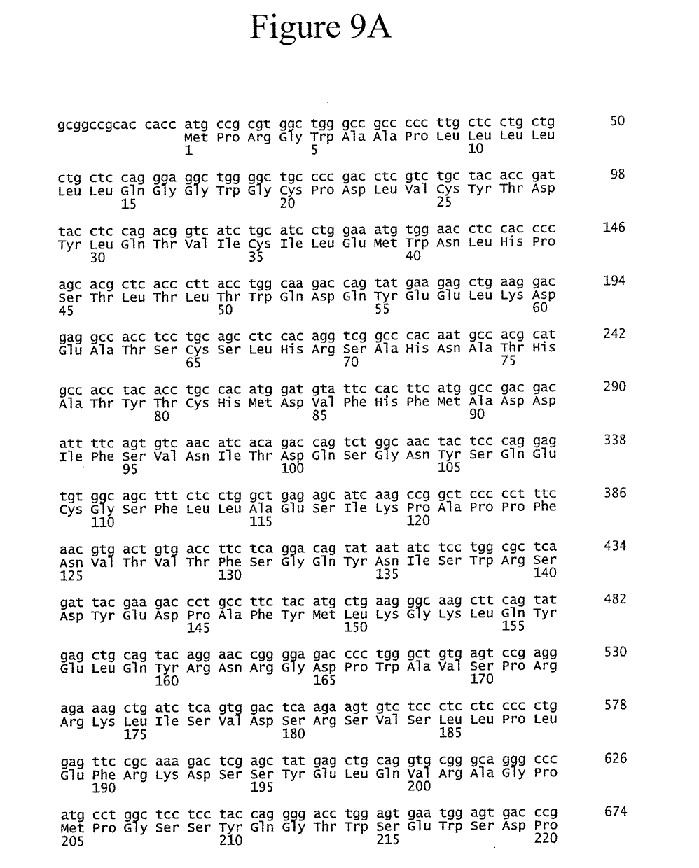

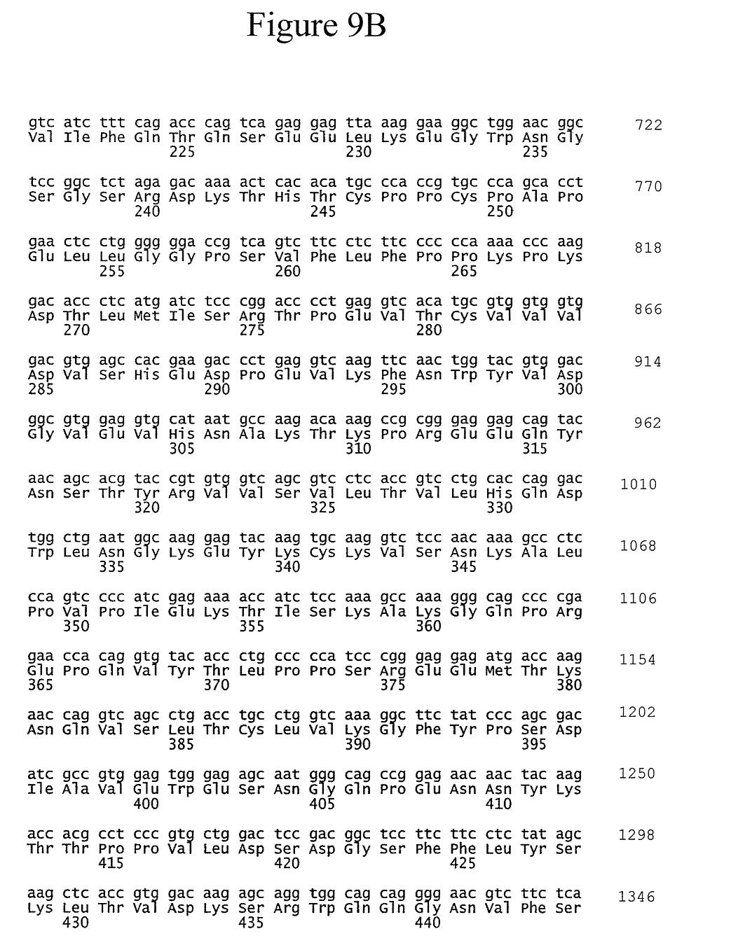

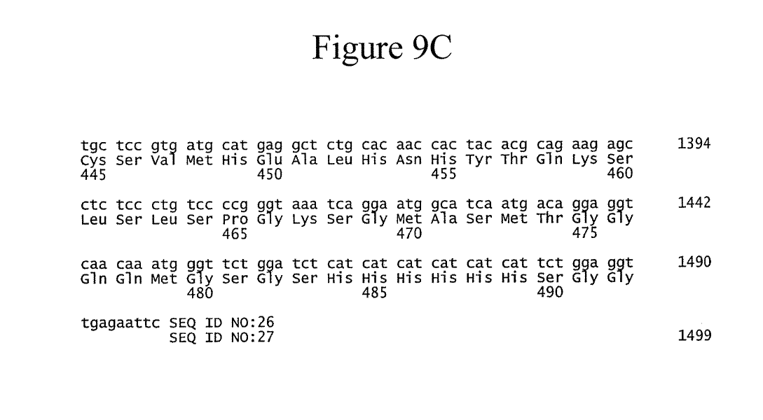

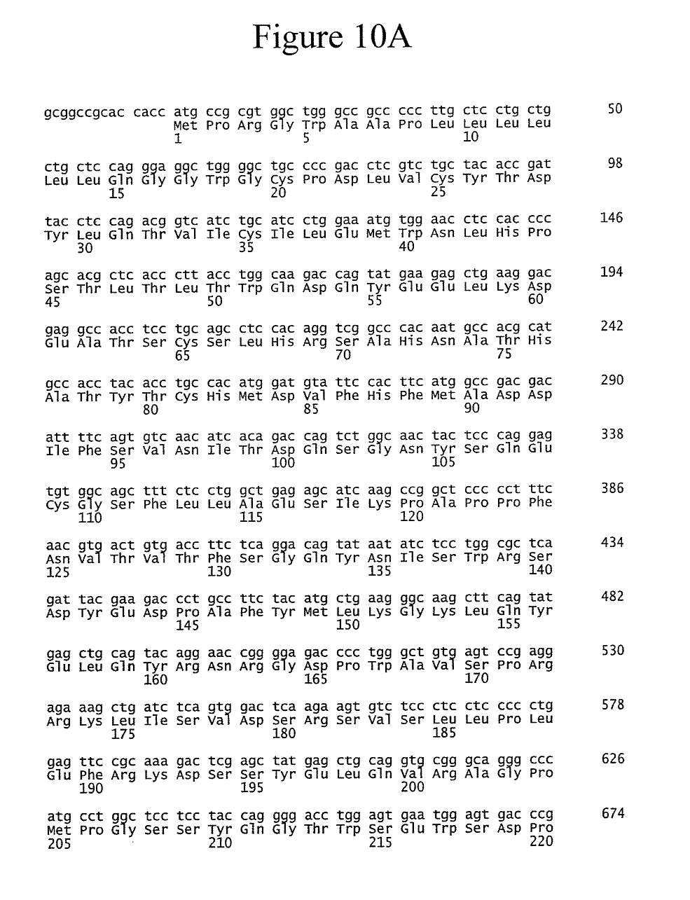

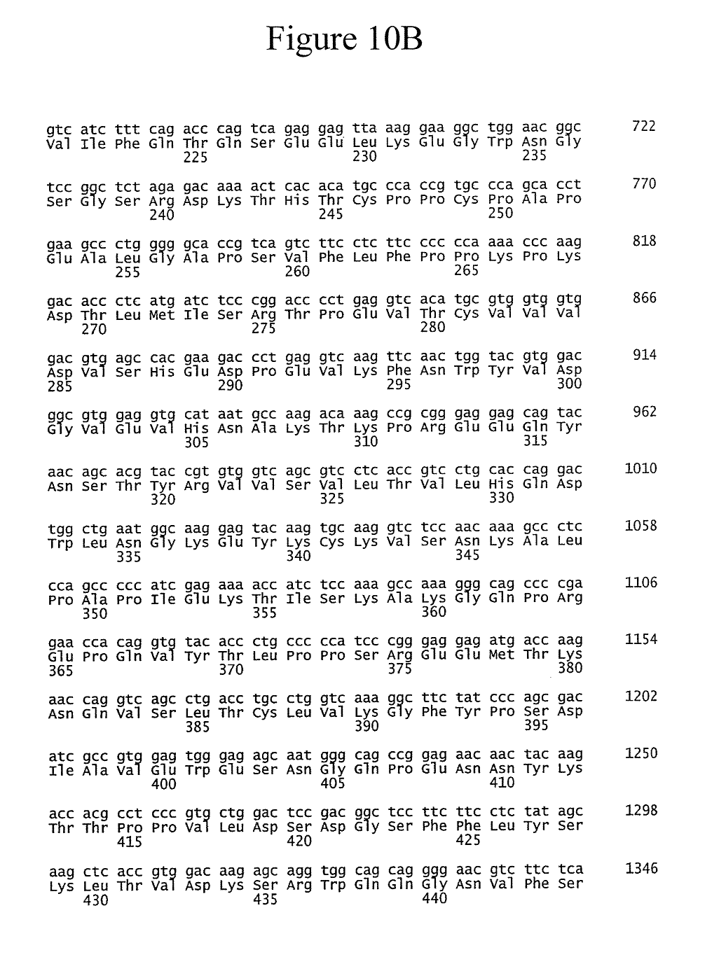

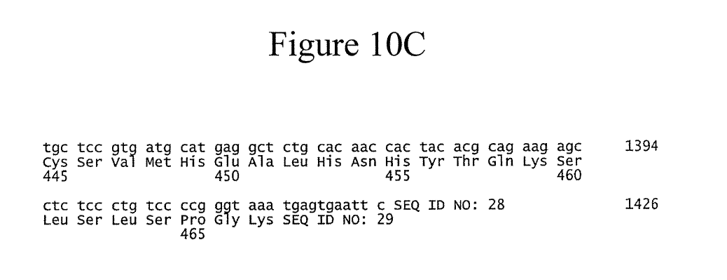

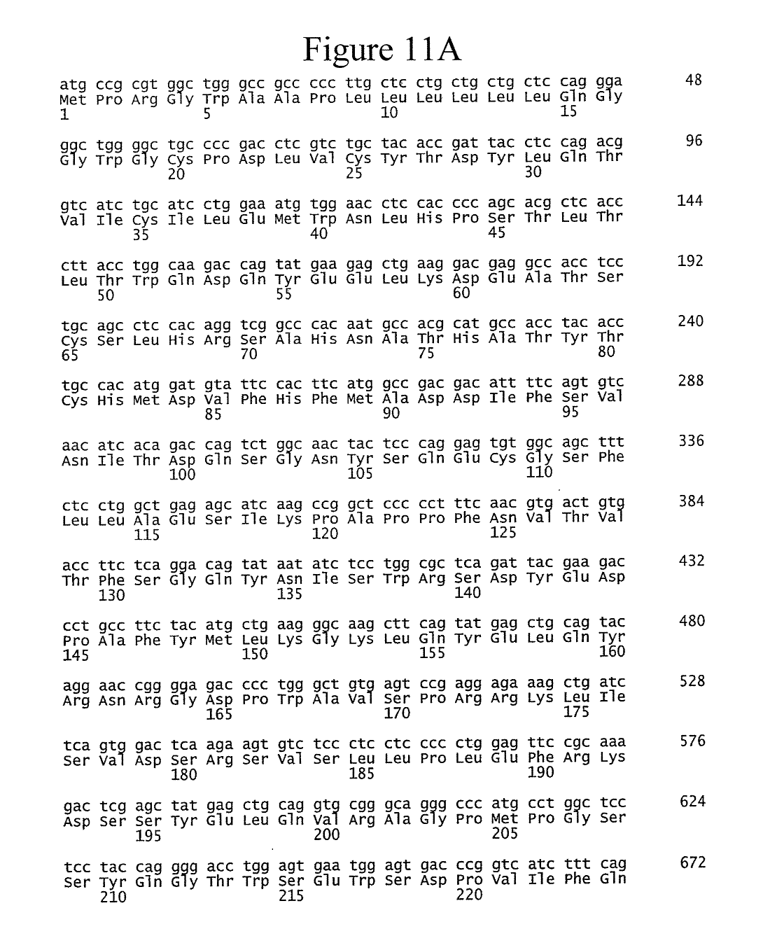

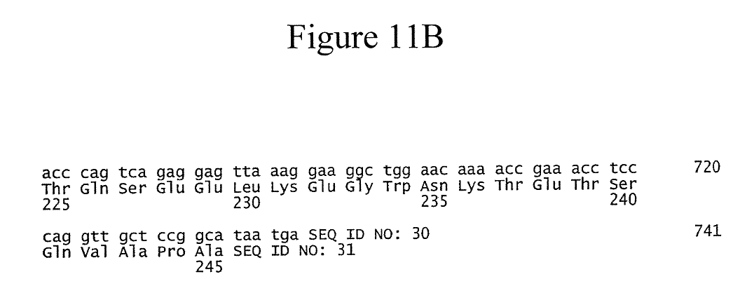

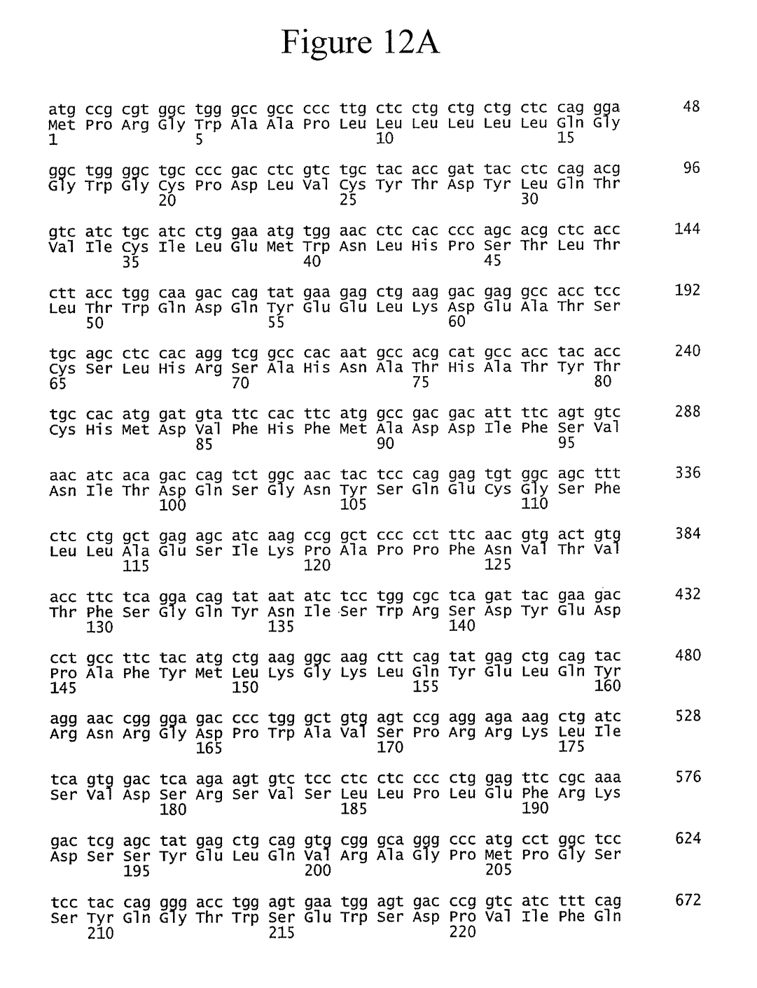

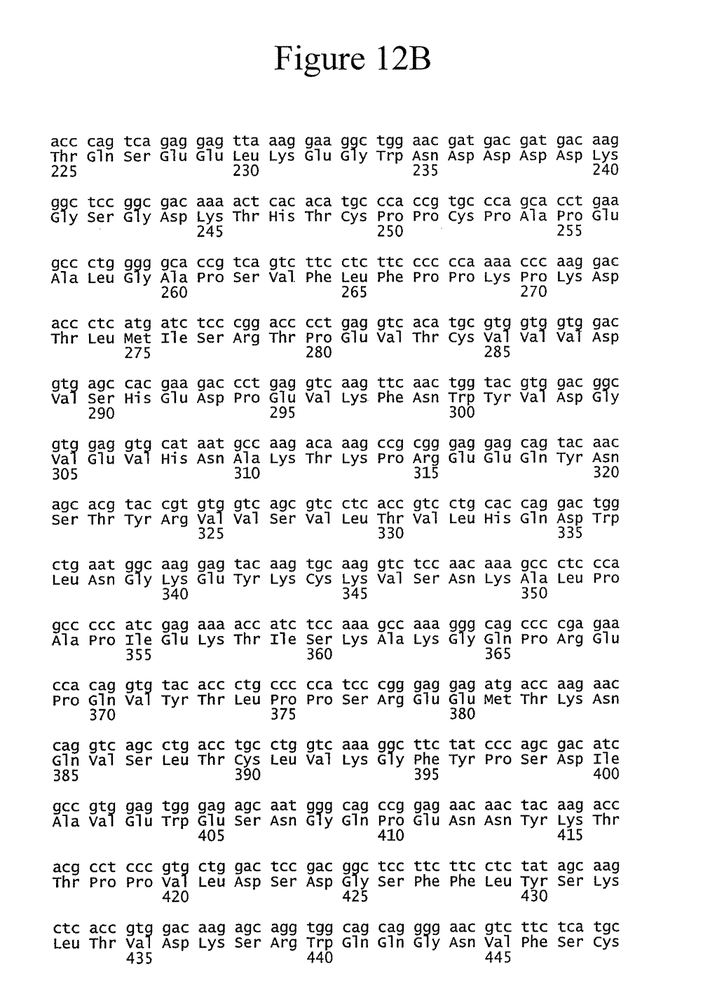

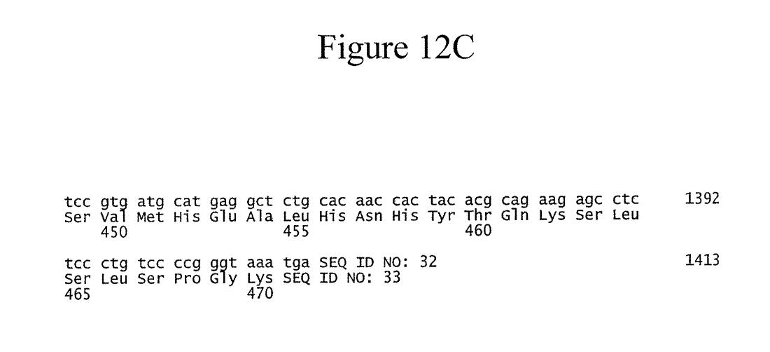

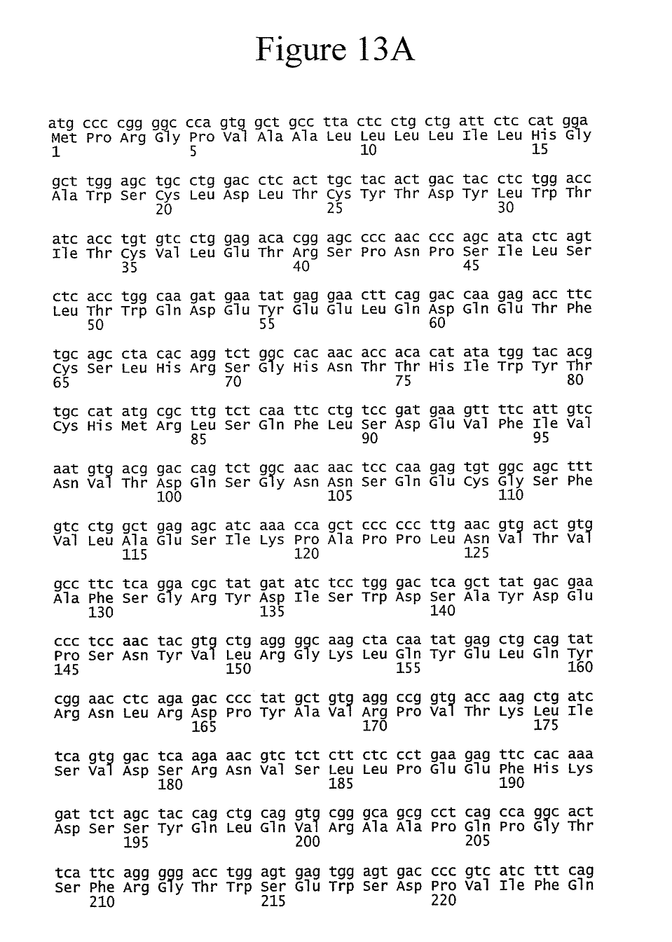

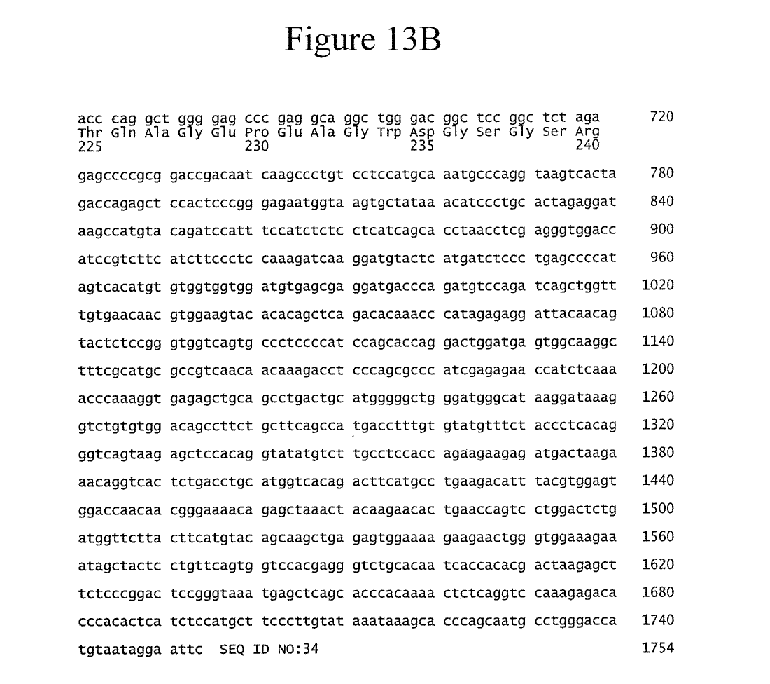

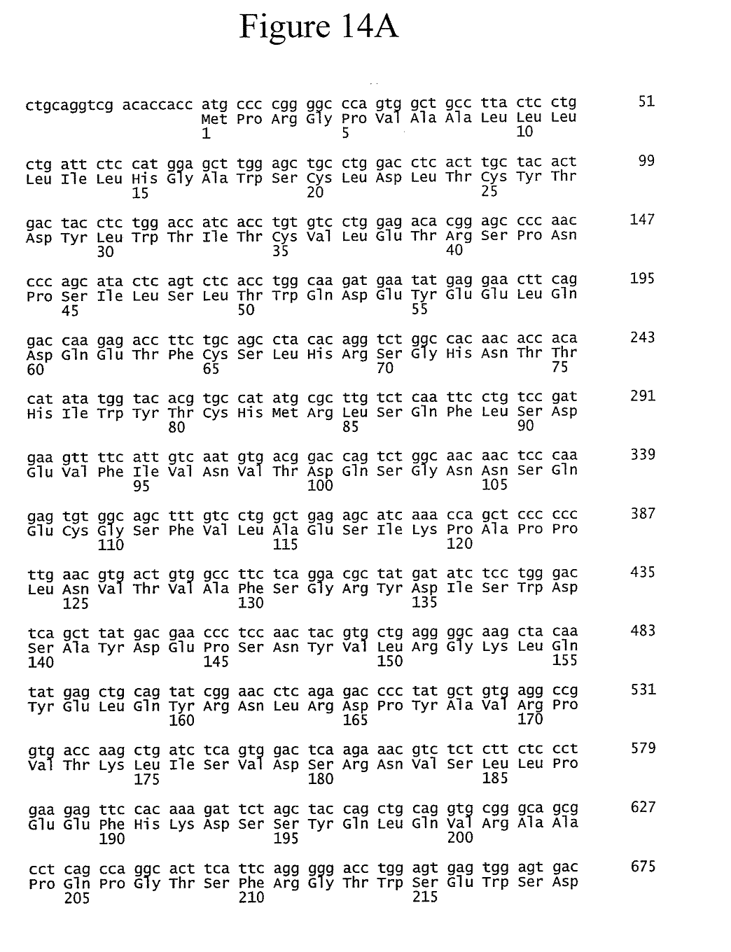

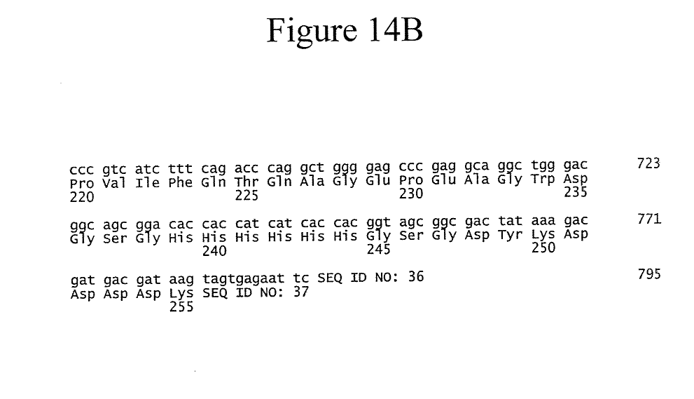

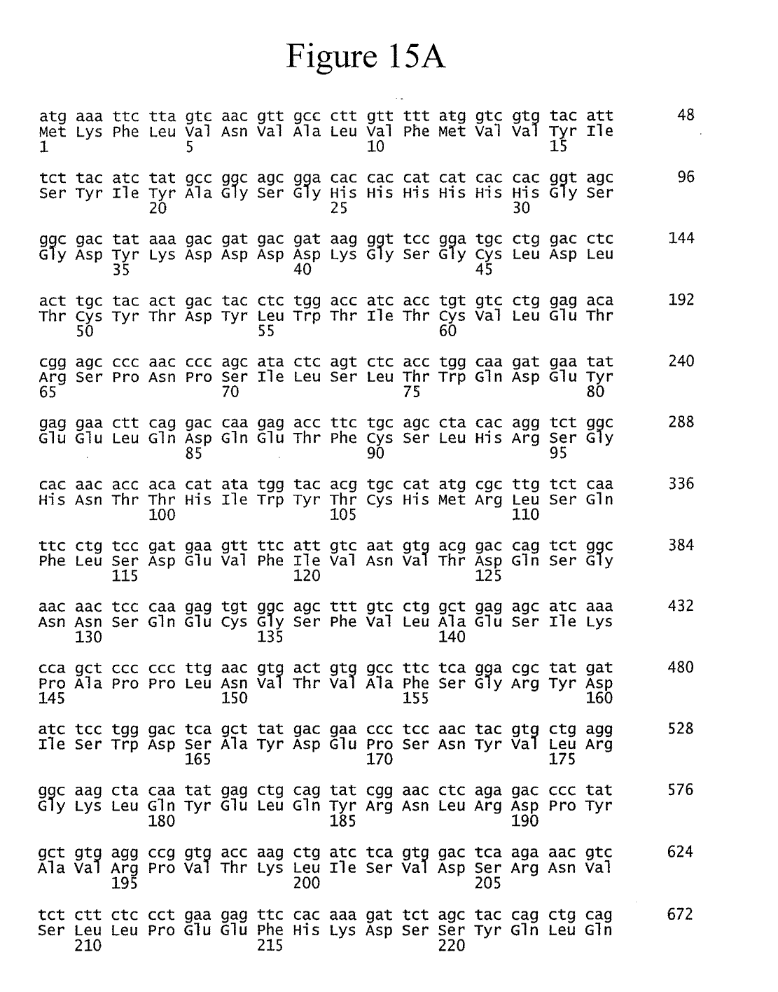

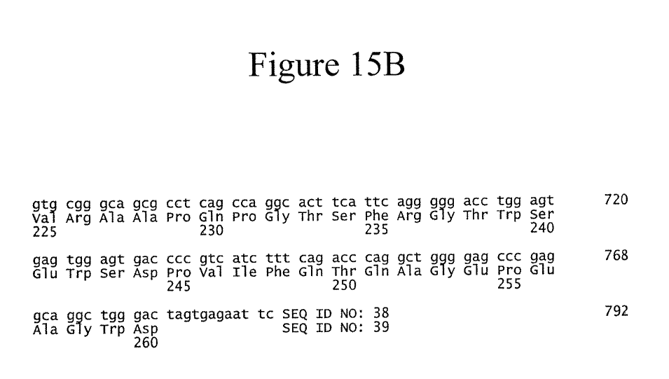

Examples of antagonistic fusion proteins that can be used in the methods of the invention are shown in FIGS. 7-15. In one embodiment, the fusion protein includes an amino acid sequence chosen from, e.g., SEQ ID NO:23, SEQ ID NO:25, SEQ ID NO:27, SEQ ID NO:29, SEQ ID NO:31, SEQ ID NO:33, SEQ ID NO:35, SEQ ID NO:37, or SEQ ID NO:39, or a sequence at least 85%, 90%, 95%, 98% or more identical thereto. In other embodiments, the fusion protein includes an amino acid sequence encoded by a nucleotide sequence chosen from, e.g., SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38, or a sequence at least 85%, 90%, 95%, 98% or more identical thereto. Preferred fusion proteins have the amino acid sequence shown as SEQ ID NO:25 or SEQ ID NO:29 (FIGS. 8A-8C and 10A-10C, respectively), or a sequence at least 85%, 90%, 95%, 98% or more identical thereto. In other embodiments, the fusion protein includes an amino acid sequence encoded by a nucleotide sequence chosen from, e.g., SEQ ID NO:24 or SEQ ID NO:28 (FIGS. 8A-8C and 10A-10C, respectively), or a sequence at least 85%, 90%, 95%, 98% or more identical thereto. Most preferably, the fusion protein has the amino acid sequence shown as SEQ ID NO:29, or has an amino acid sequence encoded by a nucleotide sequence shown as SEQ ID NO:28 (FIG. 10A-10C).

In other embodiments, the IL-21/IL-21R antagonists are antibodies, or antigen-binding fragments thereof, that bind to IL-21 or IL-21R, preferably, mammalian (e.g., human or murine) IL-21 or IL-21R.

MU-1 proteins of the invention may also be used to immunize animals to obtain polyclonal and monoclonal antibodies which specifically react with the MU-1 protein and which may inhibit binding of ligands to the receptor. Such antibodies may be obtained using the entire MU-1 as an immunogen, or by using fragments of MU-1. Smaller fragments of the MU-1 may also be used to immunize animals. The peptide immunogens additionally may contain a cysteine residue at the carboxyl terminus and are conjugated to a hapten such as keyhole limpet hemocyanin (KLH). Additional peptide immunogens may be generated by replacing tyrosine residues with sulfated tyrosine residues. Methods for synthesizing such peptides are well known in the art.

Neutralizing or nonneutralizing antibodies (preferably monoclonal antibodies) binding to MU-1 protein may also be useful in the treatment of conditions described above. These neutralizing monoclonal antibodies may be capable of blocking ligand binding to the MU-1 receptor chain.

The present invention further provides for compositions comprising an antibody that specifically reacts with an IL-21 or an IL-21R.

Human monoclonal antibodies (mAbs) directed against IL-21 or IL-21R can be generated using transgenic mice carrying the human immunoglobulin genes, rather than the mouse system. Splenocytes from these transgenic mice immunized with the antigen of interest are used to produce hybridomas that secrete human mAbs with specific affinities for epitopes from a human protein (see, e.g., Wood et al., International Publication WO 91/00906; Kucherlapati et al., International Publication WO 91/10741; Lonberg et al., International Publication WO 92/03918; Kay et al., International Publication WO 92/03917; Lonberg et al. (1994) Nature 368:856-59; Green et al. (1994) Nat. Genet. 7:13-21; Morrison et al. (1994) Proc. Natl. Acad. Sci. U.S.A. 81:6851-55; Bruggeman et al. (1993) Year Immunol. 7:33-40; Tuaillon et al. (1993) Proc. Natl. Acad. Sci. U.S.A. 90:3720-24; Bruggeman et al. (1991) Eur. J. Immunol. 21:1323-1326).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies; this method is well known in the art. After immunizing an animal with an immunogen, the antibody repertoire of the resulting B cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al. (1991) Biotechniques 11:152-56). A similar strategy can also been used to amplify human heavy and light chain variable regions from human antibodies (Larrick et al. (1991) Methods: Companion to Methods in Enzymology 2:106-10).

Chimeric antibodies, including chimeric immunoglobulin chains, can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see, e.g., Robinson et al., International Patent Publication PCT/US86/02269; Akira et al., European Patent Application 184,187; Taniguchi, European Patent Application 171,496; Morrison et al., European Patent Application 173,494; Neuberger et al., International Publication WO 86/01533; Cabilly et al. U.S. Pat. No. 4,816,567; Cabilly et al., European Patent Application 125,023; Better et al. (1988) Science 240:1041-43; Liu et al. (1987) Proc. Natl. Acad. Sci. U.S.A. 84:3439-43; Liu et al. (1987) J. Immunol. 139:3521-26; Sun et al. (1987) Proc. Natl. Acad. Sci. U.S.A. 84:214-18; Nishimura et al. (1987) Canc. Res. 47:999-1005; Wood et al. (1985) Nature 314:446-49; Shaw et al. (1988) J. Natl. Cancer Inst. 80:1553-59).

An antibody or an immunoglobulin chain can be humanized by methods known in the art. Humanized antibodies, including humanized immunoglobulin chains, can be generated by replacing sequences of the Fv variable region that are not directly involved in antigen binding with equivalent sequences from human Fv variable regions. General methods for generating humanized antibodies are provided by Morrison (1985) Science 229:1202-07; Oi et al. (1986) BioTechniques 4:214; and Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acids are well known to those skilled in the art and, for example, may be obtained from a hybridoma producing an antibody against a predetermined target. The recombinant DNA encoding the humanized antibody, or fragment thereof, can then be cloned into an appropriate expression vector.

Humanized or CDR-grafted antibody molecules or immunoglobulins can be produced by CDR-grafting or CDR substitution, wherein one, two, or all CDRs of an immunoglobulin chain can be replaced (see, e.g., U.S. Pat. No. 5,225,539; Jones et al. (1986) Nature 321:552-25; Verhoeyan et al. (1988) Science 239:1534; Beidler et al. (1988) J. Immunol. 141:4053-60; Winter, U.S. Pat. No. 5,225,539, the contents of all of which are hereby incorporated by reference. Winter describes a CDR-grafting method that may be used to prepare the humanized antibodies of the present invention (U.K. Patent Application GB 2188638A, filed on Mar. 26, 1987; Winter U.S. Pat. No. 5,225,539, the contents of which are hereby incorporated by reference). All of the CDRs of a particular human antibody may be replaced with at least a portion of a nonhuman CDR or only some of the CDRs may be replaced with nonhuman CDRs. It is only necessary to replace the number of CDRs required for binding of the humanized antibody to a predetermined antigen.

Monoclonal, chimeric and humanized antibodies, which have been modified by, e.g., deleting, adding, or substituting other portions of the antibody, e.g., the constant region, are also within the scope of the invention. For example, an antibody can be modified by: (i) deleting the constant region; (ii) replacing the constant region with another constant region, e.g., a constant region meant to increase half-life, stability or affinity of the antibody, or a constant region from another species or antibody class; or (iii) modifying one or more amino acids in the constant region to alter, for example, the number of glycosylation sites, effector cell function, Fc receptor (FcR) binding, complement fixation, among others.

Methods for altering an antibody constant region are known in the art. Antibodies with altered function, e.g. altered affinity for an effector ligand, such as FcR on a cell, or the C1 component of complement, can be produced by replacing at least one amino acid residue in the constant portion of the antibody with a different residue (see, e.g., E.P. 388,151 A1, U.S. Pat. No. 5,624,821 and U.S. Pat. No. 5,648,260, the contents of all of which are hereby incorporated by reference). Similar types of alterations could be described that, if applied to the murine or other species immunoglobulin, would reduce or eliminate these functions.

For example, it is possible to alter the affinity of an Fc region of an antibody (e.g., an IgG, such as a human IgG) for an FcR (e.g., Fc gamma R1), or for C1q binding, by replacing the specified residue(s) with a residue(s) having an appropriate functionality on its side chain, or by introducing a charged functional group, such as glutamate or aspartate, or perhaps an aromatic nonpolar residue such as phenylalanine, tyrosine, tryptophan or alanine (see, e.g., U.S. Pat. No. 5,624,821).

Amino acid sequences of IL-21 polypeptides are publicly known. For example, the nucleotide sequence and amino acid sequence of a human IL-21 is available at GENBANK® Acc. No. X_{—}011082. The disclosed human IL-21 nucleotide sequence is presented below:

(SEQ ID NO: 18)

| | |
|---|---|
| 1 | gctgaagtga aaacgagacc aaggtctagc tctactgttg |
| | gtacttatga gatccagtcc |
| 61 | tggcaacatg gagaggattg tcatctgtct gatggtcatc |
| | ttcttgggga cactggtcca |
| 121 | caaatcaagc tcccaaggtc aagatcgcca catgattaga |
| | atgcgtcaac ttatagatat |
| 181 | tgttgatcag ctgaaaaatt atgtgaatga cttggtccct |
| | gaatttctgc cagctccaga |
| 241 | agatgtagag acaaactgtg agtggtcagc tttttcctgc |
| | tttcagaagg cccaactaaa |
| 301 | gtcagcaaat acaggaaaca atgaaaggat aatcaatgta |
| | tcaattaaaa agctgaagag |
| 361 | gaaaccacct tccacaaatg cagggagaag acagaaacac |
| | agactaacat gcccttcatg |
| 421 | tgattcttat gagaaaaaac cacccaaaga attcctagaa |
| | agattcaaat cacttctcca |
| 481 | aaagatgatt catcagcatc tgtcctctag aacacacgga |
| | agtgaagatt cctgaggatc |
| 541 | taacttgcag ttggacacta tgttacatac tctaatatag |
| | tagtgaaagt catttctttg |
| 601 | tattccaagt ggaggag |

The amino acid sequence of the disclosed human IL-21 polypeptide is presented below:

(SEQ ID NO: 19)

MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS

The invention also encompasses nucleic acids that hybridize to the nucleotide sequence set forth in SEQ ID NO:1, SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38, under highly stringent conditions (for example, 0.1×SSC at 65° C.). Isolated polynucleotides which encode MU-1 proteins or fusion proteins, but which differ from the nucleotide sequence set forth in SEQ ID NO:1, SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38, by virtue of the degeneracy of the genetic code are also encompassed by the present invention. Variations in the nucleotide sequence as set forth in SEQ ID NO:1, SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38, which are caused by point mutations or by induced modifications are also included in the invention.

The isolated polynucleotides of the invention may be operably linked to an expression control sequence, such as the pMT2 or pED expression vectors disclosed in Kaufman et al. (1991) Nucleic Acids Res. 19:4485-90, in order to produce the MU-1 protein recombinantly. Many suitable expression control sequences are known in the art. General methods of expressing recombinant proteins are also known and are exemplified in Kaufman (1990) Methods in Enzymology 185:537-66. As defined herein “operably linked” means enzymatically or chemically ligated to form a covalent bond between the isolated polynucleotide of the invention and the expression control sequence, in such a way that the MU-1 protein is expressed by a host cell which has been transformed (transfected) with the ligated polynucleotide/expression control sequence.

The term “vector”, as used herein, is intended to refer to a nucleic acid molecule capable of transporting another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “regulatory sequence” is intended to include promoters, enhancers and other expression control elements (e.g., polyadenylation signals) that control the transcription or translation of the antibody chain genes. Such regulatory sequences are described, for example, in Goeddel (1990) Gene Expression Technology: Methods in Enzymology 185, Academic Press, San Diego, Calif. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from FF-1a promoter and BGH poly A, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. Nos. 5,168,062; 4,510,245; 4,968,615.

The recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

A number of types of cells may act as suitable host cells for expression of the MU-1 protein or fusion protein thereof. Any cell type capable of expressing functional MU-1 protein may be used. Suitable mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK, Rat2, BaF3, 32D, FDCP-1, PC12, M1x or C2C12 cells.

The MU-1 protein or fusion protein thereof may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif. (e.g., the MAXBAC® kit), and such methods are well known in the art, as described in Summers and Smith, Texas Agricultural Experiment Station Bulletin No. 1555 (1987), incorporated herein by reference. Soluble forms of the MU-1 protein may also be produced in insect cells using appropriate isolated polynucleotides as described above.

Alternatively, the MU-1 protein or fusion protein thereof may be produced in lower eukaryotes such as yeast or in prokaryotes such as bacteria. Suitable yeast strains include Saccharomyces cerevisiae, Schizosaccharomyces pombe, Kluyveromyces strains, Candida, or any yeast strain capable of expressing heterologous proteins. Suitable bacterial strains include Escherichia coli, Bacillus subtilis, Salmonella typhimurium, or any bacterial strain capable of expressing heterologous proteins.

Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent. When cysteine residues are present in the primary amino acid sequence of the protein, it is often necessary to accomplish the refolding in an environment that allows correct formation of disulfide bonds (a redox system). General methods of refolding are disclosed in Kohno (1990) Meth. Enzym. 185:187-95; E.P. 0433225 and U.S. Pat. No. 5,399,677 describe other appropriate methods.

The MU-1 protein or fusion protein thereof may also be expressed as a product of transgenic animals, e.g., as a component of the milk of transgenic cows, goats, pigs, or sheep which are characterized by somatic or germ cells containing a polynucleotide sequence encoding the MU-1 protein or fusion protein thereof.

The MU-1 protein or fusion protein thereof may be prepared by growing culture transformed host cells under culture conditions necessary to express the desired protein. The resulting expressed protein may then be purified from the culture medium or cell extracts. Soluble forms of the MU-1 protein or fusion protein thereof can be purified from conditioned media. Membrane-bound forms of MU-1 protein of the invention can be purified by preparing a total membrane fraction from the expressing cell and extracting the membranes with a nonionic detergent such as TRITON® X-100.

The MU-1 protein or fusion protein can be purified using methods known to those skilled in the art. For example, the MU-1 protein of the invention can be concentrated using a commercially available protein concentration filter, for example, an AMICON® or PELLICON® ultrafiltration unit (Millipore, Billerica, Mass.). Following the concentration step, the concentrate can be applied to a purification matrix such as a gel filtration medium. Alternatively, an anion exchange resin can be employed, for example, a matrix or substrate having pendant diethylaminoethyl (DEAE) or polyethyleneimine (PEI) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred (e.g., S-SEPHAROSE® columns). The purification of the MU-1 protein or fusion protein from culture supernatant may also include one or more column steps over such affinity resins as concanavalin A-agarose, heparin-TOYOPEARL® or Cibacron blue 3GA SEPHAROSE®; or by hydrophobic interaction chromatography using such resins as phenyl ether, butyl ether, or propyl ether; or by immunoaffinity chromatography. Finally, one or more reverse-phase high performance liquid chromatography (RP-HPLC) steps employing hydrophobic RP-HPLC media, e.g., silica gel having pendant methyl or other aliphatic groups, can be employed to further purify the MU-1 protein. Affinity columns including antibodies to the MU-1 protein can also be used in purification in accordance with known methods. Some or all of the foregoing purification steps, in various combinations or with other known methods, can also be employed to provide a substantially purified isolated recombinant protein. Preferably, the isolated MU-1 protein is purified so that it is substantially free of other mammalian proteins.

MU-1 proteins or fusion proteins of the invention may also be used to screen for agents that are capable of binding to MU-1. Binding assays using a desired binding protein, immobilized or not, are well known in the art and may be used for this purpose using the MU-1 protein of the invention. Purified cell-based or protein-based (cell free) screening assays may be used to identify such agents. For example, MU-1 protein may be immobilized in purified form on a carrier and binding or potential ligands to purified MU-1 protein may be measured.

### Pharmaceutical Compositions

IL-21/IL-21R-antagonists may be used as a pharmaceutical composition when combined with a pharmaceutically acceptable carrier. Such a composition may contain, in addition to the IL-21/IL-21R-antagonists and carrier, various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration.

The pharmaceutical composition of the invention may be in the form of a liposome in which an IL-21/IL-21R-antagonist(s) is combined, in addition to other pharmaceutically acceptable carriers, with amphipathic agents such as lipids that exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, e.g., in U.S. Pat. Nos. 4,235,871; 4,501,728; 4,837,028; and 4,737,323, all of which are incorporated herein by reference.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of an IL-21/IL-21R-antagonist is administered to a subject, e.g., mammal (e.g., a human). An IL-21/IL-21R-antagonist(s) may be administered in accordance with the method of the invention either alone or in combination with other therapies as described in more detail herein. When coadministered with one or more agents, an IL-21- and/or IL-21R-antagonist may be administered either simultaneously with the second agent, or sequentially. If administered sequentially, the attending physician will decide on the appropriate sequence of administering the IL-21/IL-21R-antagonist(s) in combination with other agents.

Administration of an IL-21/IL-21R-antagonist used in the pharmaceutical composition or to practice the method of the present invention can be carried out in a variety of conventional ways, such as oral ingestion, inhalation, or cutaneous, subcutaneous, or intravenous injection. Intravenous administration to the patient is preferred.

When a therapeutically effective amount of an IL-21/IL-21R-agonist or antagonist is administered orally, the binding agent will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% binding agent, and preferably from about 25 to 90% binding agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the binding agent, and preferably from about 1 to 50% the binding agent.

When a therapeutically effective amount of an IL-21/IL-21R-antagonist is administered by intravenous, cutaneous or subcutaneous injection, binding agent will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to binding agent an isotonic vehicle such as sodium chloride injection, Ringer's injection, dextrose injection, dextrose and sodium chloride injection, lactated Ringer's injection, or other vehicles as known in the art. The pharmaceutical composition of the present invention may also contain stabilizers, preservatives, buffers, antioxidants, or other additive known to those of skill in the art.

The amount of an IL-21/IL-21R-antagonist in the pharmaceutical composition of the present invention will depend upon the nature and severity of the condition being treated, and on the nature of prior treatments that the patient has undergone. Ultimately, the attending physician will decide the amount of binding agent with which to treat each individual patient. Initially, the attending physician will administer low doses of binding agent and observe the patient's response. Larger doses of binding agent may be administered until the optimal therapeutic effect is obtained for the patient, and at that point the dosage is not generally increased further. It is contemplated that the various pharmaceutical compositions used to practice the method of the present invention should contain about 0.1 μg to about 100 mg IL-21/IL-21R-antagonist per kg body weight.

The duration of intravenous therapy using the pharmaceutical composition of the present invention will vary, depending on the severity of the disease being treated and the condition and potential idiosyncratic response of each individual patient. It is contemplated that the duration of each application of the IL-21/IL-21R-antagonist will be in the range of 12 to 24 hours of continuous intravenous administration. Ultimately the attending physician will decide on the appropriate duration of intravenous therapy using the pharmaceutical composition of the present invention.

The polynucleotides and proteins of the present invention are expected to exhibit one or more of the uses or biological activities (including those associated with assays cited herein) identified herein. Uses or activities described for proteins of the present invention may be provided by administration or use of such proteins or by administration or use of polynucleotides encoding such proteins (such as, for example, in gene therapies or vectors suitable for introduction of DNA).

### Uses of IL-21/IL-21R Antagonists to Decrease Immune Cell Activity

In yet another aspect, the invention features a method for inhibiting the activity of an immune cell, e.g., mature T cells (mature CD8+ T cells, mature CD4+ T cells), mature NK cells, B cells, macrophages and megakaryocytes, or a population thereof, by contacting a population of T cells with an IL-21/IL-21R antagonist in an amount sufficient to inhibit the activity of the immune cell or population. Antagonists of IL-21 and/or IL-21R (e.g., a fusion protein or a neutralizing antibody, as described herein) can also be administered to subjects for which inhibition of an immune response is desired. These conditions or disorders include, e.g., autoimmune disorders (e.g., arthritic disorders, RA, IBD), SLE, asthma, glomerulonephritis, psoriasis, or graft/organ transplantation (and rejection related thereto).

Applicants have shown that a reduction of IL-21R activity by using a neutralizing fusion protein that includes the extracellular domain of the IL-21R fused to an Fc immunoglobulin region ameliorates inflammatory symptoms in collagen-induced arthritis (CIA) animal models (Example 7), as well as animal models for Crohn's disease, ulcerative colitis, and IBD (Examples 9 and 11), graft rejection (Example 10), psoriasis (Example 11), and lupus (Example 13). Expression of IL-21R mRNA is upregulated in the paws of CIA mice (Example 8). Mice deficient in IL-21R show a reduction in antigen-induced airway inflammation (Example 12). Accordingly, IL-21R binding agents that antagonize IL-21/IL-21R activity can be used to induce immune suppression in vivo, e.g., for treating or preventing immune cell-associated pathologies, including autoimmune disorders (e.g., arthritic disorders, RA, IBD), SLE, glomerulonephritis, asthma, psoriasis, or graft/organ transplantation.

The IL-21R DNA also maps to the chromosomal locus for Crohn's disease, thus providing additional support for the use of IL-21/IL-21R antagonists to treat Crohn's disease and other inflammatory bowel diseases.

The subject method can also be used to modulate (e.g., inhibit) the activity, e.g., proliferation, differentiation, survival, of an immune cell, and, thus, can be used to treat or prevent a variety of immune disorders. Nonlimiting examples of the disorders that can be treated or prevented include, but are not limited to, transplant rejection, autoimmune diseases (including, for example, diabetes mellitus, arthritis (including RA, juvenile RA, osteoarthritis (OA), psoriatic arthritis), multiple sclerosis, encephalomyelitis, myasthenia gravis, SLE, glomerulonephritis, autoimmune thyroiditis, dermatitis (including atopic dermatitis and eczematous dermatitis), psoriasis and related skin conditions (e.g., conditions associated with UV damage, e.g., photoaging, atopic dermatitis, cutaneous T cell lymphoma such as mycosis fangoides, allergic and irritant contact dermatitis, lichen planus, alopecia areata, vitiligo, ocular cicatricial pemphigoid, and urticaria), Sjogren's syndrome, Crohn's disease, aphthous ulcer, iritis, ulcerative colitis, spondyoarthropathy, ankylosing spondylitis, intrinsic asthma, allergic asthma, chronic obstructive pulmonary disease (COPD), interstitial lung fibrosis, cutaneous lupus erythematosus, scleroderma, drug eruptions, autoimmune uveitis, allergic encephalomyelitis, Wegener's granulomatosis, hepatitis, Stevens-Johnson syndrome, idiopathic sprue, Graves' disease, sarcoidosis, liver fibrosis, primary biliary cirrhosis, uveitis posterior, graft-versus-host disease, and allergy, such as atopic allergy. Preferred disorders that can be treated using the IL-21/IL-21R antagonists include arthritic disorders (e.g., RA, juvenile RA, OA, psoriatic arthritis, and ankylosing spondylitis (preferably, rheumatoid arthritis)), multiple sclerosis, type I diabetes, lupus (SLE), IBD (Crohn's disease, ulcerative colitis), asthma, vasculitis, allergy, scleroderma, glomerulonephritis and psoriasis.

In another embodiment, IL-21/IL-21R antagonists, alone or in combination with other therapeutic agents as described herein (e.g., TNF antagonists), can be used to treat multiple myeloma and related B lymphocytic malignancies (Brenne et al. (2002) Blood 99(10):3756-62).

Using the IL-21/IL-21R antagonists, it is possible to modulate immune responses in a number of ways. Downregulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, nonantigen-specific, process that requires continuous exposure of the T cells to the suppressive agent. Tolerance, which involves inducing nonresponsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Downregulating or preventing immune functions, e.g., using IL-21/IL-21R antagonists, will be useful in situations of tissue, skin and organ transplantation and in graft-versus-host disease (GVHD). For example, inhibiting T cell function may reduce tissue destruction in tissue transplantation. Typically, in tissue transplants, rejection of the transplant is initiated through its recognition as foreign by T cells, followed by an immune reaction that destroys the transplant. The administration of an IL-21/IL-21R antagonist, alone or in combination with a molecule which inhibits or blocks interaction of other immune effectors prior to, during, or following transplantation, can serve to reduce immune responses.

The efficacy of IL-21/IL-21R antagonists in preventing organ transplant rejection or GVHD can be assessed using animal models that are predictive of efficacy and dosing in humans. Examples of appropriate systems which can be used include allogeneic cardiac grafts in rats and xenogeneic pancreatic islet cell grafts in mice, both of which have been used to examine the immunosuppressive effects of CTLA4 Ig fusion proteins in vivo, as described in Lenschow et al. (1992) Science 257:789-92 and Turka et al. (1992) Proc. Natl. Acad. Sci U.S.A., 89:11102-05. IL-21/IL-21R antagonists can also be evaluated in other animal models, e.g., in murine models for vascularized cardiac allografts, and full thickness skin allografts. The model can test rejection of tissues that have full MHC mismatches, and can combine IL-21 blockade with donor specific lymphocyte transfusion. In addition, murine models of GVHD (see, e.g., Paul ed., Fundamental Immunology, Raven Press, New York (1989) pp. 846-47) can be used to determine the effect of IL-21/IR-21R antagonists in vivo on the development of GVHD or SLE. The efficacy of IL-21/IL-21R antagonists in preventing organ transplant rejection or GVHD can also be assessed in combination with other therapeutic agents, e.g., an immunosuppressant, such as rapamycin, cyclosporine, or CTLA4 Ig.

IL-21/IL-21R antagonists may also be therapeutically useful for treating autoimmune diseases. Many autoimmune disorders are the result of inappropriate activation of T cells that are reactive against self tissue and that promote the production of cytokines and autoantibodies involved in the pathology of the diseases. Preventing the activation of autoreactive T cells may reduce or eliminate disease symptoms. Administration of IL-21/IL-21R antagonists, alone or in combination with other agents (e.g., as described herein) can be used to inhibit T cell activation and prevent production of autoantibodies or T cell-derived cytokines that may be involved in the disease process. Additionally, IL-21/IL-21R antagonists, alone or in combination with other agents (e.g., as described herein) increase antigen-specific tolerance of autoreactive T cells and lead to long-term relief from the disease. The efficacy of these agents in preventing or alleviating autoimmune disorders can be determined using a number of well-characterized animal models of human autoimmune diseases. Examples include murine experimental autoimmune encephalitis, systemic lupus erythematosus in MRL/lpr/lpr mice or NZB hybrid mice, murine autoimmune collagen arthritis, diabetes mellitus in NOD mice and BB rats, and murine experimental myasthenia gravis (see, e.g., Paul ed., Fundamental Immunology, Raven Press, New York (1989) pp. 840-56).

In one embodiment, the IL-21/IL-21R antagonists, e.g., pharmaceutical compositions thereof, are administered in combination therapy, i.e., combined with other agents, e.g., therapeutic agents, which are useful for treating pathological conditions or disorders, such as immune and inflammatory disorders. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment or in the subject.

For example, the combination therapy can include one or more IL-21/IL-21R antagonists, e.g., an antibody or an antigen-binding fragment thereof (e.g., a chimeric, humanized, human, or in vitro-generated antibody or antigen-binding fragment thereof) against IL-21 or IL-21 receptor, an IL-21 fusion protein, a soluble IL-21 receptor, peptide inhibitor or a small molecule inhibitor) coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail herein. Furthermore, one or more IL-21/IL-21R antagonists described herein may be used in combination with two or more of the therapeutic agents described herein. Such combination therapies may advantageously utilize lower dosages of the administered therapeutic agents, thus avoiding possible toxicities or complications associated with the various monotherapies. Moreover, the therapeutic agents disclosed herein act on pathways that differ from the IL-21/IL-21R receptor pathway, and thus are expected to enhance and/or synergize with the effects of the IL-21/IL-21R antagonists.

Preferred therapeutic agents used in combination with an IL-21/IL-21R antagonist are those agents that interfere at different stages in the autoimmune and subsequent inflammatory response. In one embodiment, one or more IL-21/IL-21R antagonists described herein may be coformulated with, and/or coadministered with, one or more additional agents such as other cytokine or growth factor antagonists (e.g., soluble receptors, peptide inhibitors, small molecules, ligand fusions); or antibodies or antigen-binding fragments thereof that bind to other targets (e.g., antibodies that bind to other cytokines or growth factors, their receptors, or other cell surface molecules); and anti-inflammatory cytokines or agonists thereof. Nonlimiting examples of the agents that can be used in combination with the IL-21/IL-21R antagonists described herein, include, but are not limited to, antagonists of one or more interleukins (ILs) or their receptors, e.g., antagonists of IL-1, IL-2, IL-6, IL-7, IL-8, IL-12, IL-13, IL-15, IL-16, IL-18, and IL-22; antagonists of cytokines or growth factors or their receptors, such as tumor necrosis factor (TNF), LT, EMAP-II, GM-CSF, FGF and PDGF. IL-21/IL-21R antagonists can also be combined with inhibitors of, e.g., antibodies to, cell surface molecules such as CD2, CD3, CD4, CD8, CD25, CD28, CD30, CD40, CD45, CD69, CD80 (B7.1), CD86 (B7.2), CD90, or their ligands, including CD154 (gp39 or CD40L), or LFA-1/ICAM-1 and VLA-4/VCAM-1 (Yusuf-Makagiansar et al. (2002) Med. Res. Rev. 22(2):146-67). Preferred antagonists that can be used in combination with IL-21/IL-21R antagonists described herein include antagonists of IL-1, IL-6, IL-12, TNFα, IL-15, IL-17, IL-18, and IL-22.

Examples of those agents include IL-12 antagonists, such as chimeric, humanized, human or in vitro-generated antibodies (or antigen-binding fragments thereof) that bind to IL-12 (preferably human IL-12), e.g., the antibody disclosed in WO 00/56772, Genetics Institute/BASF); IL-12 receptor inhibitors, e.g., antibodies to human IL-12 receptor; and soluble fragments of the IL-12 receptor, e.g., human IL-12 receptor. Examples of IL-6 antagonists include antibodies (or antigen-binding fragments thereof) against IL-6 or its receptor, e.g., chimeric, humanized, human or in vitro-generated antibodies to human IL-6 or its receptor, soluble fragments of the IL-6 receptor, and IL-6-binding proteins. Examples of IL-15 antagonists include antibodies (or antigen-binding fragments thereof) against IL-15 or its receptor, e.g., chimeric, humanized, human or in vitro-generated antibodies to human IL-15 or its receptor, soluble fragments of the IL-15 receptor, and IL-15-binding proteins. Examples of IL-18 antagonists include antibodies, e.g., chimeric, humanized, human or in vitro-generated antibodies (or antigen-binding fragments thereof), to human IL-18, soluble fragments of the IL-18 receptor, and IL-18 binding proteins (IL-18BP, Mallat et al. (2001) Circ. Res. 89:e41-45). Examples of IL-1 antagonists include interleukin-1-converting enzyme (ICE) inhibitors, such as Vx740, IL-1 antagonists, e.g., IL-1RA (ANIKINRA™, Amgen), sIL1RII (Immunex), and anti-IL-1 receptor antibodies (or antigen-binding fragments thereof).

Examples of TNF antagonists include chimeric, humanized, human or in vitro-generated antibodies (or antigen-binding fragments thereof) to TNF (e.g., human TNFα), such as D2E7, (human TNFα antibody, U.S. Pat. No. 6,258,562; BASF), CDP-571/CDP-870/BAY-10-3356 (humanized anti-TNFα antibody; Celltech/Pharmacia), cA2 (chimeric anti-TNFα antibody; REMICADE™, Centocor); anti-TNF antibody fragments (e.g., CPD870); soluble fragments of the TNF receptors, e.g., p55 or p75 human TNF receptors or derivatives thereof, e.g., 75 kdTNFR-IgG (75 kDa TNF receptor-IgG fusion protein, ENBREL™; Immunex; see, e.g., Arthritis & Rheumatism (1994) Vol. 37, S295; J. Invest. Med. (1996) Vol. 44, 235A), p55 kdTNFR-IgG (55 kDa TNF receptor-IgG fusion protein (Lenercept)); enzyme antagonists, e.g., TNFα converting enzyme (TACE) inhibitors (e.g., an alpha-sulfonyl hydroxamic acid derivative, WO 01/55112, and N-hydroxyformamide TACE inhibitor GW 3333, -005, or -022); and TNF-bp/s-TNFR (soluble TNF binding protein; see, e.g., Arthritis & Rheumatism (1996) Vol. 39, No. 9 (supplement), S284; Amer. J. Physiol.—Heart and Circulatory Physiology (1995) Vol. 268, pp. 37-42). Preferred TNF antagonists are soluble fragments of the TNF receptors, e.g., p55 or p75 human TNF receptors or derivatives thereof, e.g., 75 kdTNFR-IgG, and TNF-α converting enzyme (TACE) inhibitors.

In other embodiments, the IL-21-/IL-21R antagonists described herein can be administered in combination with one or more of the following: IL-13 antagonists, e.g., soluble IL-13 receptors (sIL-13) and/or antibodies against IL-13; IL-2 antagonists, e.g., DAB 486-IL-2 and/or DAB 389-IL-2 (IL-2 fusion proteins; Seragen; see, e.g., Arthritis & Rheumatism (1993) Vol. 36, 1223), and/or antibodies to IL-2R, e.g., anti-Tac (humanized anti-IL-2R; Protein Design Labs, Cancer Res. (1990) March 1; 50(5):1495-502). Yet another combination includes IL-21 antagonists in combination with nondepleting anti-CD4 inhibitors (IDEC-CE9.1/SB 210396 (nondepleting primatized anti-CD4 antibody; IDEC/SmithKline)). Yet other preferred combinations include antagonists of the costimulatory pathway CD80 (B7.1) or CD86 (B7.2) including antibodies, soluble receptors or antagonistic ligands; as well as p-selectin glycoprotein ligand (PSGL), anti-inflammatory cytokines, e.g., IL-4 (DNAX/Schering); IL-10 (SCH 52000; recombinant IL-10 DNAX/Schering); IL-13 and TGF, and agonists thereof (e.g., agonist antibodies).

In other embodiments, one or more IL-21/IL-21R antagonists can be coformulated with, and/or coadministered with, one or more anti-inflammatory drugs, immunosuppressants, or metabolic or enzymatic inhibitors. Nonlimiting examples of the drugs or inhibitors that can be used in combination with the IL-21 antagonists described herein, include, but are not limited to, one or more of: nonsteroidal anti-inflammatory drug(s) (NSAIDs), e.g., ibuprofen, tenidap (see, e.g., Arthritis & Rheumatism (1996) Vol. 39, No. 9 (supplement), S280)), naproxen (see, e.g., Neuro Report (1996) Vol. 7, pp. 1209-1213), meloxicam, piroxicam, diclofenac, and indomethacin; sulfasalazine (see, e.g., Arthritis & Rheumatism (1996) Vol. 39, No. 9 (supplement), S281); corticosteroids such as prednisolone; cytokine suppressive anti-inflammatory drug(s) (CSAIDs); inhibitors of nucleotide biosynthesis, e.g., inhibitors of purine biosynthesis, folate antagonists (e.g., methotrexate (N-[4-[[(2,4-diamino-6-pteridinyl)methyl]methylamino]benzoyl]-L-glutamic acid); and inhibitors of pyrimidine biosynthesis, e.g., dihydroorotate dehydrogenase (DHODH) inhibitors (e.g., leflunomide (see, e.g., Arthritis & Rheumatism (1996) Vol. 39, No. 9 (supplement), S131; Inflammation Research (1996) Vol. 45, pp. 103-107). Preferred therapeutic agents for use in combination with IL-21/IL-21R antagonists include NSAIDs, CSAIDs, (DHODH) inhibitors (e.g., leflunomide), and folate antagonists (e.g., methotrexate).

Examples of additional inhibitors include one or more of: corticosteroids (oral, inhaled and local injection); immunosuppressants, e.g., cyclosporin, tacrolimus (FK-506); and mTOR inhibitors, e.g., sirolimus (rapamycin) or rapamycin derivatives, e.g., soluble rapamycin derivatives (e.g., ester rapamycin derivatives, e.g., CCI-779 (Elit (2002) Current Opinion Investig. Drugs 3(8):1249-53; Huang et al. (2002) Current Opinion Investig. Drugs 3(2):295-304); agents which interfere with signaling by proinflammatory cytokines such as TNFα or IL-1 (e.g. IRAK, NIK, IKK, p38 or MAP kinase inhibitors); COX2 inhibitors, e.g., celecoxib and variants thereof, MK-966, see, e.g., Arthritis & Rheumatism (1996) Vol. 39, No. 9 (supplement), S81); phosphodiesterase inhibitors, e.g., R973401 (phosphodiesterase Type IV inhibitor; see, e.g., Arthritis & Rheumatism (1996) Vol. 39, No. 9 (supplement), S282); phospholipase inhibitors, e.g., inhibitors of cytosolic phospholipase 2 (cPLA2) (e.g., trifluoromethyl ketone analogs (U.S. Pat. No. 6,350,892)); inhibitors of vascular endothelial cell growth factor or growth factor receptor, e.g., VEGF inhibitor and/or VEGF-R inhibitor; and inhibitors of angiogenesis. Preferred therapeutic agents for use in combination with IL-21/IL-21R antagonists include immunosuppressants, e.g., cyclosporin, tacrolimus (FK-506); and mTOR inhibitors, e.g., sirolimus (rapamycin) or rapamycin derivatives, e.g., soluble rapamycin derivatives (e.g., ester rapamycin derivatives, e.g., CCI-779; COX2 inhibitors, e.g., celecoxib and variants thereof); and phospholipase inhibitors, e.g., inhibitors of cytosolic phospholipase 2 (cPLA2) (e.g., trifluoromethyl ketone analogs).

Additional examples of therapeutic agents that can be combined with an IL-21/IL-21R antagonist include one or more of: 6-mercaptopurines (6-MP); azathioprine sulphasalazine; mesalazine; olsalazine chloroquine/hydroxychloroquine; pencillamine; aurothiomalate (intramuscular and oral); azathioprine; colchicine; beta-2 adrenoreceptor agonists (salbutamol, terbutaline, salmeteral); xanthines (theophylline, aminophylline); cromoglycate; nedocromil; ketotifen; ipratropium and oxitropium; mycophenolate mofetil; adenosine agonists; antithrombotic agents; complement inhibitors; and adrenergic agents.

The use of the IL-21/IL-21R antagonists disclosed herein in combination with other therapeutic agents to treat or prevent specific immune disorders is discussed in further detail herein.

Nonlimiting examples of agents for treating or preventing arthritic disorders (e.g., RA, inflammatory arthritis, juvenile RA, OA and psoriatic arthritis), with which an IL-21/IL-21R antagonist can be combined include one or more of the following: IL-12 antagonists as described herein, NSAIDs; CSAIDs; TNFs, e.g., TNFα, antagonists as described herein; nondepleting anti-CD4 antibodies as described herein; IL-2 antagonists as described herein; anti-inflammatory cytokines, e.g., IL-4, IL-10, IL-13 and TGFα, or agonists thereof; IL-1 or IL-1 receptor antagonists as described herein); phosphodiesterase inhibitors as described herein; COX-2 inhibitors as described herein; Iloprost (see, e.g., Arthritis & Rheumatism (1996) Vol. 39, No. 9 (supplement), S82); methotrexate; thalidomide (see, e.g., Arthritis & Rheumatism (1996) Vol. 39, No. 9 (supplement), S282) and thalidomide-related drugs (e.g., Celgen); leflunomide; inhibitor of plasminogen activation, e.g., tranexamic acid (see, e.g., Arthritis & Rheumatism (1996) Vol. 39, No. 9 (supplement), S284); cytokine inhibitor, e.g., T-614; see, e.g., Arthritis & Rheumatism (1996) Vol. 39, No. 9 (supplement), S282); prostaglandin E1 (see, e.g., Arthritis & Rheumatism (1996) Vol. 39, No. 9 (supplement), S282); azathioprine (see, e.g., Arthritis & Rheumatism (1996) Vol. 39, No. 9 (supplement), S281); an inhibitor of interleukin-1 converting enzyme (ICE); zap-70 and/or lck inhibitor (inhibitor of the tyrosine kinase zap-70 or lck); an inhibitor of vascular endothelial cell growth factor or vascular endothelial cell growth factor receptor as described herein; an inhibitor of angiogenesis as described herein; corticosteroid anti-inflammatory drugs (e.g., SB203580); TNF-convertase inhibitors; interleukin-11 (see, e.g., Arthritis & Rheumatism (1996) Vol. 39, No. 9 (supplement), S296); IL-13 (see, e.g., Arthritis & Rheumatism (1996) Vol. 39, No. 9 (supplement), S308); IL-17 inhibitors (see, e.g., Arthritis & Rheumatism (1996) Vol. 39, No. 9 (supplement), S120); gold; penicillamine; chloroquine; hydroxychloroquine; chlorambucil; cyclophosphamide; cyclosporine; total lymphoid irradiation; anti-thymocyte globulin; CD5-toxins; orally-administered peptides and collagen; lobenzarit disodium; Cytokine Regulating Agents (CRAs) HP228 and HP466 (Houghten Pharmaceuticals, Inc.); ICAM-1 antisense phosphorothioate oligodeoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline; ante IL-2R antibodies; marine and botanical lipids (fish and plant seed fatty acids; see, e.g., DeLuca et al. (1995) Rheum. Dis. Clin. North Am. 21:759-777); auranofin; phenylbutazone; meclofenamic acid; flufenamic acid; intravenous immune globulin; zileuton; mycophenolic acid (RS-61443); tacrolimus (FK-506); sirolimus (rapamycin); amiprilose (therafectin); cladribine (2-chlorodeoxyadenosine); and azaribine. Preferred combinations include one or more IL-21 antagonists in combination with methotrexate or leflunomide, and in moderate or severe rheumatoid arthritis cases, cyclosporine.

Preferred examples of inhibitors to use in combination with IL-21/IL-21R antagonists to treat arthritic disorders include TNF antagonists (e.g., chimeric, humanized, human or in vitro-generated antibodies, or antigen-binding fragments thereof, that bind to TNF; soluble fragments of a TNF receptor, e.g., p55 or p75 human TNF receptor or derivatives thereof, e.g., 75 kdTNFR-IgG (75 kDa TNF receptor-IgG fusion protein, ENBREL™), p55 kDa TNF receptor-IgG fusion protein; TNF enzyme antagonists, e.g., TNFα converting enzyme (TACE) inhibitors); antagonists of IL-6, IL-12, IL-15, IL-17, IL-18, IL-22; T cell and B cell depleting agents (e.g., anti-CD4 or anti-CD22 antibodies); small molecule inhibitors, e.g., methotrexate and leflunomide; sirolimus (rapamycin) and analogs thereof, e.g., CCI-779; Cox-2 and cPLA2 inhibitors; NSAIDs; p38 inhibitors, TPL-2, Mk-2 and NFκb inhibitors; RAGE or soluble RAGE; P-selectin or PSGL-1 inhibitors (e.g., small molecule inhibitors, antibodies thereto, e.g., antibodies to P-selectin); estrogen receptor beta (ERB) agonists or ERB-NFkb antagonists. Most preferred additional therapeutic agents that can be coadministered and/or coformulated with one or more IL-21/IL-21R antagonists include one or more of: a soluble fragment of a TNF receptor, e.g., p55 or p75 human TNF receptor or derivatives thereof, e.g., 75 kdTNFR-IgG (75 kDa TNF receptor-IgG fusion protein, ENBREL™); methotrexate, leflunomide, or a sirolimus (rapamycin) or an analog thereof, e.g., CCI-779.

Nonlimiting examples of agents for treating or preventing multiple sclerosis with which an IL-21-/IL-21R antagonist can be combined include the following: interferons, e.g., interferon-alphala (e.g., AVONEX™; Biogen) and interferon-1b (BETASERON™; Chiron/Berlex); Copolymer 1 (Cop-1; COPAXONE™; Teva Pharmaceutical Industries, Inc.); hyperbaric oxygen; intravenous immunoglobulin; clabribine; TNF antagonists as described herein; corticosteroids; prednisolone; methylprednisolone; azathioprine; cyclophosphamide; cyclosporine; methotrexate; 4-aminopyridine; and tizanidine. Additional antagonists that can be used in combination with IL-21 include antibodies to or antagonists of other human cytokines or growth factors, for example, TNF, LT, IL-1, IL-2, IL-6, IL-7, IL-8, IL-12, IL-15, IL-16, IL-18, EMAP-11, GM-CSF, FGF, and PDGF. IL-21 antagonists as described herein can be combined with antibodies to cell surface molecules such as CD2, CD3, CD4, CD8, CD25, CD28, CD30, CD40, CD45, CD69, CD80, CD86, CD90 or their ligands. The IL-21 antagonists may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents that interfere with signaling by proinflammatory cytokines as described herein, IL-1b converting enzyme inhibitors (e.g., Vx740), anti-P7s, PSGL, TACE inhibitors, T cell signaling inhibitors such as kinase inhibitors, metalloproteinase inhibitors, sulfasalazine, azathloprine, 6-mercaptopurines, angiotensin converting enzyme inhibitors, soluble cytokine receptors and derivatives thereof, as described herein, and anti-inflammatory cytokines (e.g. IL-4, IL-10, IL-13 and TGF).

Preferred examples of therapeutic agents for multiple sclerosis with which the IL-21 antagonists can be combined include interferon-b, for example, IFNβ-1a and IFNβ-1b; copaxone, corticosteroids, IL-1 inhibitors, TNF inhibitors, antibodies to CD40 ligand and CD80, IL-12 antagonists.

Nonlimiting examples of agents for treating or preventing inflammatory bowel disease (Crohn's disease; ulcerative colitis) with which an IL-21/IL-21R antagonist can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies; growth factors; elastase inhibitors; pyridinyl-imidazole compounds; TNF antagonists as described herein; IL-4, IL-10, IL-13 and/or TGFb cytokines or agonists thereof (e.g., agonist antibodies); IL-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of platelet activating factor (PAF); ciprofloxacin; and lignocaine.

In one embodiment, an IL-21/IL-21R antagonist can be used in combination with one or more antibodies directed at other targets involved in regulating immune responses, e.g., transplant rejection, graft-vs-host disease, or other immune response-related disorders. Nonlimiting examples of agents for treating or preventing immune responses with which an IL-21/IL-21R antagonist of the invention can be combined include the following: antibodies against cell surface molecules or their ligands, including but not limited to CD25 (IL-2 receptor-a), CD11a (LFA-1), CD54 (ICAM-1), CD4, CD40, CD40L, CD45, CD28/CTLA4, CD80 (B7-1) and/or CD86 (B7-2). In yet another embodiment, an IL-21/IL-21R antagonist can be used in combination with corticosteroids; sirolimus (rapamycin) and analogs thereof, e.g., CCI-779; cyclosporin A; FK506; FTY720; azathioprine; cyclophosphamide; methotrexate; anti-IL-2R antibodies, e.g., basiliximab, daclizumab; cA2 (chimeric anti-TNFα antibody; REMICADE™, Centocor); anti-CD3 antibodies (e.g., muromonab-CD3); Copolymer 1 (Cop-1; COPAXONE™; Teva Pharmaceutical Industries, Inc.); deoxyspergualin; and mycophenolate mofetil.

Nonlimiting examples of agents for treating or preventing psoriasis and other skin conditions with which an IL-21/IL-21R antagonist can be combined include one or more of the following: inhibitors of CD2 or LFA-3 interactions (e.g., soluble CD2- or LFA-polypeptides, such as Fc fusions, or antibodies against CD2 or LFA-3), cyclosporin A, prednisone, FK506, methotrexate, PUVA, UV light, steroids, retinoids, interferon, or nitrogen mustard. Examples of preferred agents that can be used in combination with an IL-21/IL-21R antagonist include cyclosporine A and methotrexate.

Nonlimiting examples of agents for treating or preventing asthma with which an IL-21/IL-21R antagonist can be combined include one or more of the following: inhaled bronchodilators, e.g., pirbuterol, bitolterol, metaproterenol; beta 2-adrenoceptor agonists, e.g., albuterol, terbutaline, salmeterol, formoterol; antimuscarinics, e.g., ipratropium, oxitropium; systemic corticosteroids, e.g., prednisone, prednisolone, dexamethasone; inhaled corticosteroids, e.g., fluticasone, budesonide, beclomethasone, mometasone; leukotriene antagonists, e.g., montelukast sodium, zafirlukast; mast cell stabilizers, e.g., cromolyn sodium, nedocromil; omalizumab (XOLAIR™; Genentech/Novartis); or COX-2 inhibitors, as described herein.

Nonlimiting examples of agents for treating or preventing lupus (e.g., SLE) with which an IL-21/IL-21R antagonist can be combined include one or more of the following: IL-6/IL-6R antagonists, e.g. anti-IL-6 or anti-IL-6R antibodies; NSAIDs; corticosteroids, e.g., dexamethasone, hydrocortisone, methylprednisolone, prednisolone, prednisone; azathioprine, cyclophosphamide, hydroxychloroquine, or chloroquine.

Another aspect of the present invention accordingly relates to kits for carrying out the combined administration of the IL-21/IL-21R antagonists with other therapeutic compounds. In one embodiment, the kit comprises one or more binding agents formulated in a pharmaceutical carrier, and at least one agent, e.g., therapeutic agent, formulated as appropriate, in one or more separate pharmaceutical preparations.

### Exemplary Disorders

Rheumatoid arthritis is an autoimmune inflammatory disease that causes pain, swelling, stiffness, and loss of function in the joints. Rheumatoid arthritis often presents in a symmetrical pattern. The disease can affect the wrist joints and the finger joints closest to the hand. It can also affect other parts of the body besides the joints. In addition, people with rheumatoid arthritis may have fatigue, occasional fevers, and a general malaise. Positive factors for diagnosis of rheumatoid arthritis include the “rheumatoid factor” blood antibody and citrulline antibody. IL-21/IL-21R antagonists can be useful in treating, preventing, or alleviating rheumatoid arthritis or one or more symptoms of rheumatoid arthritis.

Systemic lupus erythematosus (SLE) is an autoimmune disorder that leads to inflammation and damage to various body tissues. SLE can be mediated by self-antibodies directed against one's own DNA. Lupus can affect many parts of the body, including the joints, skin, kidneys, heart, lungs, blood vessels, and brain. Although various symptoms may present, some of the most common include extreme fatigue, painful or swollen joints (arthritis), unexplained fever, skin rashes, and kidney problems (e.g., glomerulonephritis). Exemplary symptoms of lupus include painful or swollen joints, unexplained fever, and extreme fatigue. A characteristic red skin rash may appear across the nose and cheeks. Rashes may also occur on the face and ears, upper arms, shoulders, chest, and hands. Other symptoms of lupus include chest pain, hair loss, anemia, mouth ulcers, and pale or purple fingers and toes from cold and stress. Some people also experience headaches, dizziness, depression, confusion, or seizures. Positive factors for SLE diagnosis include circulating anti-nuclear antibodies, anti-DNA antibodies, and anti-Sm antibodies. IL-21/IL-21R antagonists can be useful in treating, ameliorating (alleviating), or preventing SLE or one or more symptoms of SLE.

Ankylosing spondylitis is an autoimmune disorder that not only affects the spine, but may also affect the hips, shoulders, and knees as the tendons and ligaments around the bones and joints become inflamed, resulting in pain and stiffness. Ankylosing spondylitis tends to affect people in late adolescence or early adulthood. IL-21/IL-21R antagonists can be useful in treating, preventing, or alleviating ankylosing spondylitis, or one or more symptoms thereof.

Inflammatory bowel disease (IBD) is the general name for diseases that cause inflammation in the intestines. Two examples of inflammatory bowel disease are Crohn's disease and ulcerative colitis. IL-21/IL-21R antagonists can be useful in treating, preventing, or alleviating inflammatory bowel disease or one or more symptoms of inflammatory bowel disease.

Crohn's disease causes inflammation in the small intestine. Crohn's disease usually occurs in the lower part of the small intestine (the ileum), but it can affect any part of the digestive tract, from the mouth to the anus. The inflammation can extend deep into the lining of the affected organ, causing pain and making the intestines empty frequently, resulting in diarrhea. The most common symptoms of Crohn's disease are abdominal pain, often in the lower right area, and diarrhea. Rectal bleeding, weight loss, and fever may also occur. Bleeding may be serious and persistent, leading to anemia. Direct visualization of the bowel may be useful to determine the extent of inflammation.

Ulcerative colitis is a disease that causes inflammation and sores, called ulcers, in the lining of the large intestine. The inflammation usually occurs in the rectum and lower part of the colon, but it may affect the entire colon. Ulcerative colitis rarely affects the small intestine except for the end section, called the terminal ileum. The inflammation makes the colon empty frequently, causing diarrhea. Ulcers form in places where the inflammation has killed the cells lining the colon; the ulcers bleed and produce pus. The most common symptoms of ulcerative colitis are abdominal pain and bloody diarrhea. Patients also may experience fatigue, weight loss, loss of appetite, rectal bleeding, and loss of body fluids and nutrients. About half of patients have mild symptoms. Others suffer frequent fever, bloody diarrhea, nausea, and severe abdominal cramps. Ulcerative colitis may also cause problems such as arthritis, inflammation of the eye, liver disease (hepatitis, cirrhosis, and primary sclerosing cholangitis), osteoporosis, skin rashes, and anemia. Diagnosis of ulcerative colitis typically depends on identifying bloody stool and direct visualization of the colon.

Psoriasis is a chronic skin disease of scaling and inflammation. Psoriasis occurs when skin cells quickly rise from their origin below the surface of the skin and pile up on the surface before they have a chance to mature. Usually this movement (also called turnover) takes about a month, but in psoriasis it may occur in only a few days. In its typical form, psoriasis results in patches of thick, inflamed skin covered with silvery scales. These patches, which are sometimes referred to as plaques, usually itch or feel sore. They most often occur on the elbows, knees, other parts of the legs, scalp, lower back, face, palms, and soles of the feet, but they can occur on skin anywhere on the body. Diagnosis of psoriasis is based primarily on these characteristic symptoms. A skin biopsy can be useful in diagnosis. IL-21/IL-21R antagonists can be useful in treating, preventing, or alleviating psoriasis or one or more symptoms of psoriasis. Psoriatic arthritis occurs in some patients with psoriasis, a scaling skin disorder. Psoriatic arthritis often affects the joints at the ends of the fingers and toes and is accompanied by changes in the fingernails and toenails. Back pain may occur if the spine is involved. IL-21/IL-21R antagonists can be useful in treating, preventing, or alleviating psoriasis or one or more symptoms of psoriasis or psoriatic arthritis.

Glomerular diseases include both proliferative and nonproliferative disorders. Glomerulonephritis is a disorder presenting with intraglomerular inflammation and cell proliferation (see, e.g., Hricik et al. (1998) New Eng. J. Med. 339:888-99. Nonproliferative and sclerosing glomerulopathies include membranous glomerulopathy, diabetic nephropathy, focal segmental glomerulosclerosis, thin basement membrane disease, amyloidosis, light-chain nephropathy, HIV nephropathy, Alport's syndrome, drug-induced glomerulopathies, and minimal-change disease. The inflammation accompanying glomerular disease arises largely due to antibody-mediated glomerular injury that results from autoimmunity. Activation of humoral immunity can lead to the production of antibodies against glomerular cell surfaces (e.g., basement membranes), and circulating antigen-antibody complexes are deposited in the glomerulus, reported to contribute to glomerulonephritis pathology. Glomerular injury and glomerulonephritis thus often result from larger systemic autoimmune disorders, such as, e.g., SLE, hepatitis, and fibrotic disorders. Glomerulonephritis also may be associated with IgA nephropathy, Henoch-Schonlein purpura, infection (caused by, e.g., bacteria, virus, protozoa), vasculitides, cryoglobulinemia, inherited nephritis, granulomatosis (e.g., Wegener's granulomatosis, microscopic polyangitis, and Churg-Strauss syndrome), glomerular basement membrane disease, Goodpasture's syndrome, nephritic syndrome (as occurs with, e.g., diabetes mellitus, lupus (e.g., SLE), amyloidosis, drug use, cancer, and infection), lipodystrophy, sickle cell disease, complement deficiencies, membrane proliferative glomerulonephritis, lupus nephritis, and lupus membranous nephropathy. IL-21/IL-21R antagonists can be useful in treating, ameliorating, or preventing glomerulonephritis or one or more symptoms of glomerulonephritis, and other glomerular diseases.

IL-21/IL-21R antagonists can be used to prevent or treat tissue/graft rejection or symptoms associated with rejection, e.g., before, during, or after transplantation of an organ, tissue, or cells, e.g., heart, lung, liver, kidney, pancreas, or bone marrow. Transplant/graft rejection occurs when the immune system of the host organism raises an immune response against nonself antigens in the transplanted tissue, e.g., syngeneic, allogeneic, or xenogeneic tissue. Rejection can be mediated, for example, by antibodies, lymphocytes or both and can manifest itself in a variety of different ways, including, e.g., hyperacute rejection (e.g., during the early post-transplant period), acute rejection, and chronic rejection (generally, a slowly developing process causing a progressive decline in graft function). Rejection is often accompanied by inflammation and can result in the damage and/or failure of the transplanted tissue or organ, e.g., vasculopathy, fibrosis, or a loss of organ function. During rejection, the host may experience general discomfort, pain or swelling in the area of the transplant, and/or fever. Organ and tissue transplants can be monitored for rejection, e.g., by examination of biopsies for signs of rejection, or by assessing organ function. Histopathological signs of rejection include, e.g., increased expression of HLA class II antigens, e.g., in renal tubular cells following kidney transplantation. Liver function, e.g., can be assessed by measuring serum levels of bilirubin and hepatic enzymes, e.g., alkaline phosphatase; kidney function can be assessed, e.g., by measuring serum creatine levels.

Osteoarthritis (OA) is characterized by the breakdown of cartilage at the joints. This allows bones under the cartilage to rub together, causing pain, swelling, and loss of motion of the joint. Over time, the joint may lose its normal shape, and bone spurs or osteophytes may grow on the edges of the joint. Additionally, bits of bone or cartilage can break off and float inside the joint space causing more pain and damage. People with OA typically have joint pain and limited movement. Unlike some other forms of arthritis, OA affects only joints and not internal organs. Positive factors for diagnosis of OA include loss of cartilage as seen by X-ray. IL-21/IL-21R antagonists can be useful in treating, preventing, or alleviating OA or one or more symptoms of OA.

### Respiratory Disorders

IL-21/IL-21R antagonists can be used to treat respiratory disorders including, but not limited to, asthma (e.g., allergic and nonallergic asthma); bronchitis (e.g., chronic bronchitis); chronic obstructive pulmonary disease (COPD) (e.g., emphysema, e.g., cigarette-induced emphysema); conditions involving airway inflammation, eosinophilia, fibrosis and excess mucus production, e.g., cystic fibrosis, pulmonary fibrosis, and allergic rhinitis.

The methods for treating or preventing asthma include those for extrinsic asthma (also known as allergic asthma or atopic asthma), intrinsic asthma (also known as nonallergic asthma or nonatopic asthma) or combinations of both, which has been referred to as mixed asthma. Extrinsic or allergic asthma includes incidents caused by, or associated with, e.g., allergens, such as pollens, spores, grasses or weeds, pet danders, dust, mites, etc. As allergens and other irritants present themselves at varying points over the year, these types of incidents are also referred to as seasonal asthma. Also included in the group of extrinsic asthma is bronchial asthma and allergic bronchopulmonary aspergillosis.

Asthma that can be treated or alleviated by the present methods include those caused by infectious agents, such as viruses (e.g., cold and flu viruses, respiratory syncytial virus (RSV), paramyxovirus, rhinovirus and influenza viruses). RSV, rhinovirus and influenza virus infections are common in children, and viral infection is a leading cause of respiratory tract illnesses in infants and young children. Children with viral bronchiolitis can develop chronic wheezing and asthma, which can be treated using the methods of the invention. Also included are the asthma conditions that may be brought about in some asthmatics by exercise and/or cold air. The methods are useful for asthmas associated with smoke exposure (e.g., cigarette-induced and industrial smoke), as well as industrial and occupational exposures, such as smoke; ozone; noxious gases; sulfur dioxide; nitrous oxide; fumes, including isocyanates, from paint, plastics, polyurethanes, varnishes, etc.; wood, plant, or other organic dusts; etc. The methods are also useful for asthmatic incidents associated with food additives, preservatives, or pharmacological agents. Also included are methods for treating, inhibiting, or alleviating the types of asthma referred to as silent asthma or cough variant asthma.

The methods disclosed herein are also useful for treatment and alleviation of asthma associated with gastroesophageal reflux (GERD), which can stimulate bronchoconstriction. GERD, along with retained bodily secretions, suppressed cough, and exposure to allergens and irritants in the bedroom can contribute to asthmatic conditions and have been collectively referred to as nighttime asthma or nocturnal asthma. In methods of treatment, inhibition or alleviation of asthma associated with GERD, a pharmaceutically effective amount of the IL21/IL-21R antagonist can be used as described herein in combination with a pharmaceutically effective amount of an agent for treating GERD. These agents include, but are not limited to, proton pump inhibiting agents like PROTONIX® brand of delayed-release pantoprazole sodium tablets, PRILOSEC® brand omeprazole delayed release capsules, ACIPHEX® brand rebeprazole sodium delayed release tablets, or PREVACID® brand delayed release lansoprazole capsules.

### Atopic Disorders and Symptoms Thereof

“Atopic” refers to a group of diseases where there is often an inherited tendency to develop an allergic reaction. Examples of atopic disorders include allergy, allergic rhinitis, atopic dermatitis, and hay fever. An IL-21/IL-21R pathway antagonist can be administered to ameliorate an atopic disorder or one or more of the symptoms thereof.

Symptoms of allergic rhinitis (hay fever) include itchy, runny, sneezing, or stuffy noses, and itchy eyes. An IL-21/IL-21R pathway antagonist can be administered to ameliorate one or more of these symptoms.

Atopic dermatitis is a chronic disease that affects the skin. Information about atopic dermatitis is available, e.g., from NIH Publication No. 03-4272. In atopic dermatitis, the skin can become extremely itchy, leading to redness, swelling, cracking, weeping clear fluid, and finally, crusting and scaling. In many cases, there are periods of time when the disease is worse (called exacerbations or flares) followed by periods when the skin improves or clears up entirely (called remissions). Atopic dermatitis is often referred to as “eczema,” which is a general term for the several types of inflammation of the skin. Atopic dermatitis is the most common of the many types of eczema. Examples of atopic dermatitis include: allergic contact eczema or dermatitis (e.g., sometimes manifested as a red, itchy, weepy reaction where the skin has come into contact with a foreign substance, such as poison ivy or certain preservatives in creams and lotions); contact eczema (e.g., a localized reaction that includes redness, itching, and burning where the skin has come into contact with an allergen or with an irritant such as an acid, a cleaning agent, or other chemical); dyshidrotic eczema (e.g., an irritation of the skin on the palms of hands and soles of the feet characterized by clear, deep blisters that itch and burn); neurodermatitis (e.g., scaly patches of the skin on the head, lower legs, wrists, or forearms caused by a localized itch (such as an insect bite) that become intensely irritated when scratched ); nummular eczema (e.g., manifested as coin-shaped patches of irritated skin—most common on the arms, back, buttocks, and lower legs—that may be crusted, scaling, and extremely itchy); seborrheic eczema (e.g., manifested as yellowish, oily, scaly patches of skin on the scalp, face, and occasionally other parts of the body). Additional particular symptoms include stasis dermatitis, atopic pleat (e.g., Dennie-Morgan fold), cheilitis, hyperlinear palms, hyperpigmented eyelids: eyelids that have become darker in color from inflammation or hay fever, ichthyosis, keratosis pilaris, lichenification, papules, and urticaria. An IL-21/IL-21R pathway antagonist can be administered to ameliorate one or more of these symptoms.

### Fibrotic Disorders

Although production of collagen is a highly regulated process, its disturbance may lead to the development of tissue fibrosis. Abnormal accumulation of fibrous materials may ultimately lead to organ failure (Border et al. (1994) New Engl. J. Med. 331:1286-92). Injury to any organ leads to a stereotypical physiological response: platelet-induced hemostasis, followed by an influx of inflammatory cells and activated fibroblasts. Cytokines derived from these cell types drive the formation of new extracellular matrix and blood vessels (granulation tissue). The generation of granulation tissue is a carefully orchestrated program in which the expression of protease inhibitors and extracellular matrix proteins is upregulated, and the expression of proteases is reduced, leading to the accumulation of extracellular matrix.

The development of fibrotic conditions, whether induced or spontaneous, is caused at least in part by stimulation of fibroblast activity. The influx of inflammatory cells and activated fibroblasts into the injured organ depends on the ability of these cell types to interact with the interstitial matrix, which contains primarily collagens. Many of the diseases associated with the proliferation of fibrous tissue are both chronic and often debilitating, including for example, skin diseases such as scleroderma. Some, including pulmonary fibrosis, can be fatal due in part to the fact that the currently available treatments for this disease have significant side effects and are generally not efficacious in slowing or halting the progression of fibrosis (Nagler et al. (1996) Am. J. Respir. Crit. Care Med. 154:1082-86).

Fibrotic disorders include disorders characterized by fibrosis, e.g., fibrosis of an internal organ, a dermal fibrosing disorder, and fibrotic conditions of the eye. Fibrosis of internal organs (e.g., liver, lung, kidney, heart blood vessels, gastrointestinal tract), occurs in disorders such as pulmonary fibrosis, myelofibrosis, liver cirrhosis, mesangial proliferative glomerulonephritis, crescentic glomerulonephritis, diabetic nephropathy, renal interstitial fibrosis, renal fibrosis in patients receiving cyclosporin, and HIV associated nephropathy.

Dermal fibrosing disorders include, e.g., scleroderma, morphea, keloids, hypertrophic scars, familial cutaneous collagenoma, and connective tissue nevi of the collagen type. Fibrotic conditions of the eye include conditions such as diabetic retinopathy, postsurgical scarring (for example, after glaucoma filtering surgery and after cross-eye surgery), and proliferative vitreoretinopathy.

Additional fibrotic conditions that may be treated by the methods of the present invention include: rheumatoid arthritis, diseases associated with prolonged joint pain and deteriorated joints, systemic sclerosism (including progressive systemic sclerosis), polymyositis, dermatomyositis, eosinophilic fasciitis, morphea (localized scleroderma), Raynaud's syndrome, and nasal polyposis.

An IL-21/IL-21R pathway antagonist can be administered to treat or prevent fibrotic disorders or to ameliorate one or more of the symptoms of these disorders.

### Assays for Measuring the Activity of IL-21/IL-21R Antagonists as Modulators of Cytokine Production and Cell Proliferation/Differentiation

The activity of IL-21/IL-21R antagonists as modulators of cytokine production and cell proliferation/differentiation can be tested using any one of a number of routine factor-dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

Assays for T-cell or thymocyte proliferation include without limitation those described in: Current Protocols in Immunology, Ed. by J. E. Coligan, A. M. Kruisbeek, D. H. Margulies, E. M. Shevach, W Strober, Pub. Greene Publishing Associates and Wiley-Interscience (Chapter 3, In vitro assays for Mouse Lymphocyte Function 3.1-3.19; Chapter 7, Immunologic studies in Humans); Takai et al. (1986) J. Immunol. 137:3494-500; Bertagnolli et al. (1990) J. Immunol. 145:1706-12; Bertagnolli et al. (1991) Cellular Immunology 133:327-41; Bertagnolli et al. (1992) J. Immunol. 149:3778-83; Bowman et al. (1994) J. Immunol. 152:1756-61. Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E.e.a. Coligan eds. Vol 1 pp. 3.12.1-3.12.14, John Wiley and Sons, Toronto (1994); and Measurement of mouse and human Interferon gamma, Schreiber, R. D. In Current Protocols in Immunology. J. E.e.a. Coligan eds. Vol 1 pp. 6.8.1-6.8.8, John Wiley and Sons, Toronto (1994).

Assays for proliferation and differentiation of hematopoietic and lymphopoietic cells include, without limitation, those described in: Measurement of Human and Murine Interleukin 2 and Interleukin 4, Bottomly,. K., Davis, L. S. and Lipsky, P. E. In Current Protocols in Immunology. J. E.e.a. Coligan eds. Vol 1 pp. 6.3.1-6.3.12, John Wiley and Sons, Toronto (1991); deVries et al. (1991) J. Exp. Med. 173:1205-11; Moreau et al. (1988) Nature 336:690-92; Greenberger et al. (1983) Proc. Natl. Acad. Sci. U.S.A. 80:2931-38; Measurement of mouse and human interleukin 6, Nordan, R. In Current Protocols in Immunology. J. E.e.a. Coligan eds. Vol 1 pp. 6.6.1-6.6.5, John Wiley and Sons, Toronto (1991); Smith et al. (1986) Proc. Natl. Acad. Sci. U.S.A. 83:1857-61; Measurement of human Interleukin 11, Bennett, F., Giannotti, J., Clark, S. C. and Turner, K. J. In Current Protocols in Immunology. J. E.e.a. Coligan eds. Vol 1 pp. 6.15.1 John Wiley and Sons, Toronto (1991); Measurement of mouse and human Interleukin 9, Ciarletta, A., Giannotti, J., Clark, S. C. and Turner, K. J. In Current Protocols in Immunology. J. E.e.a. Coligan eds. Vol 1 pp. 6.13.1, John Wiley and Sons, Toronto (1991).

Assays for T cell clone responses to antigens (which will identify, among others, proteins that affect APC-T cell interactions as well as direct T cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In vitro assays for Mouse Lymphocyte Function; Chapter 6, Cytokines and their cellular receptors; Chapter 7, Immunologic studies in Humans); Weinberger et al. (1980) Proc. Natl. Acad. Sci. U.S.A. 77:6091-95; Weinberger et al. (1981) Eur. J. Immun. 11:405-11; Takai et al. (1986) J. Immunol. 137:3494-500; Takai et al. (1988) J. Immunol. 140:508-12.

### EXAMPLES

The invention will be further illustrated in the following nonlimiting examples.

### Example 1

### Isolation and Characterization of Murine MU-1 cDNAs

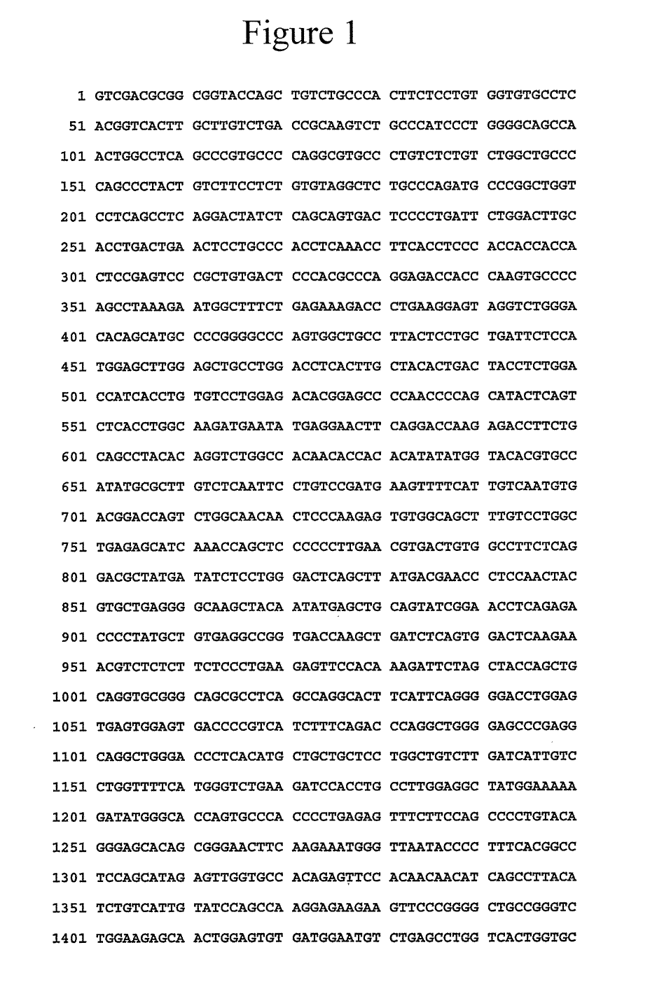

A partial fragment of the murine homolog of the MU-1 receptor was isolated by PCR using oligonucleotides derived from the human sequences. cDNA was prepared from RNA isolated from 17-day old murine thymus and from the murine 2D6 T cell line. A DNA fragment of approximately 300 nucleotides was amplified from the cDNA by PCR with the following oligonucleotides, corresponding to regions 584-603 and 876-896, respectively, of the human cDNA sequence in FIG. 1 (corresponding to SEQ ID NO:1):

| | | |
|---|---|---|
| AGCATCAAGCCGGCTCCCCC | (5p) | (SEQ ID NO: 11) |
| CTCCATTCACTCCAGGTCCC | (3p). | (SEQ ID NO: 12) |

Amplification was carried out using Taq polymerase in 1× Taq buffer containing 1.5 mM of magnesium chloride for 30 cycles at 94° C. for one minute, 50° C. for 1 minute, and 72° C. for one minute. The DNA sequence of this fragment was determined, and two oligonucleotides were derived from an internal portion of this fragment with the following sequences:

| | | |
|---|---|---|
| TTGAACGTGACTGRGGCCTT | (5P) | (SEQ ID NO: 13) |
| TGAATGAAGTGCCTGGCTGA | (3P). | (SEQ ID NO: 14) |

The oligonucleotides were used to amplify an internal 262-nucleotide fragment of the original PCR product (corresponding to nucleotides 781-1043 in of the murine cDNA sequence of FIG. 1, and SEQ ID NO:9) to use as a hybridization probe to screen a cDNA library isolated from the 2D6 T cell line. Filters were hybridized at 65° C. using standard 5×SSC hybridization conditions and washed into SSC at 65° C. Twenty clones were isolated that hybridized to the probe in a screen of 426,000 clones. DNA sequence was determined from two independent clones. Full-length sequence of clone #6 confirmed that it was the full-length murine homolog of human MU-1 (SEQ ID NO:9).

The full-length nucleotide sequence of murine MU-1 is shown in FIG. 1 (corresponding to SEQ ID NO:9). The nucleotide sequence has a predicted leader sequence at nucleotides 407-464, coding sequence at nucleotides 407-1993, termination codon at nucleotides 1994-1996. Nucleotides 1-406 correspond to the 5′ untranslated region, and nucleotides 1997-2628 correspond to the 3′ untranslated region (SEQ ID NO:9).

The predicted protein sequence of murine MU-1 is shown in FIG. 2 (corresponding to SEQ ID NO:10). This murine MU-1 protein contains a predicted leader sequence determined by SPScan (score=10.1) (corresponding to amino acids 1-19 of SEQ ID NO:10), and a predicted transmembrane domain (corresponding to amino acids 237-253 of SEQ ID NO:10). Predicted signaling motifs include the following regions in FIG. 2B: Box 1: amino acids 265-274 of SEQ ID NO:10; Box 2: amino acids 310-324 of SEQ ID NO:10, six tyrosine residues at positions 281, 319, 361, 368, 397, and 510 of SEQ ID NO:10. Potential STAT docking sites include: STAT5: EDDGYPA (SEQ ID NO:20); STAT3: YLQR.

### Example 2

### Comparison of Human and Murine MU-1

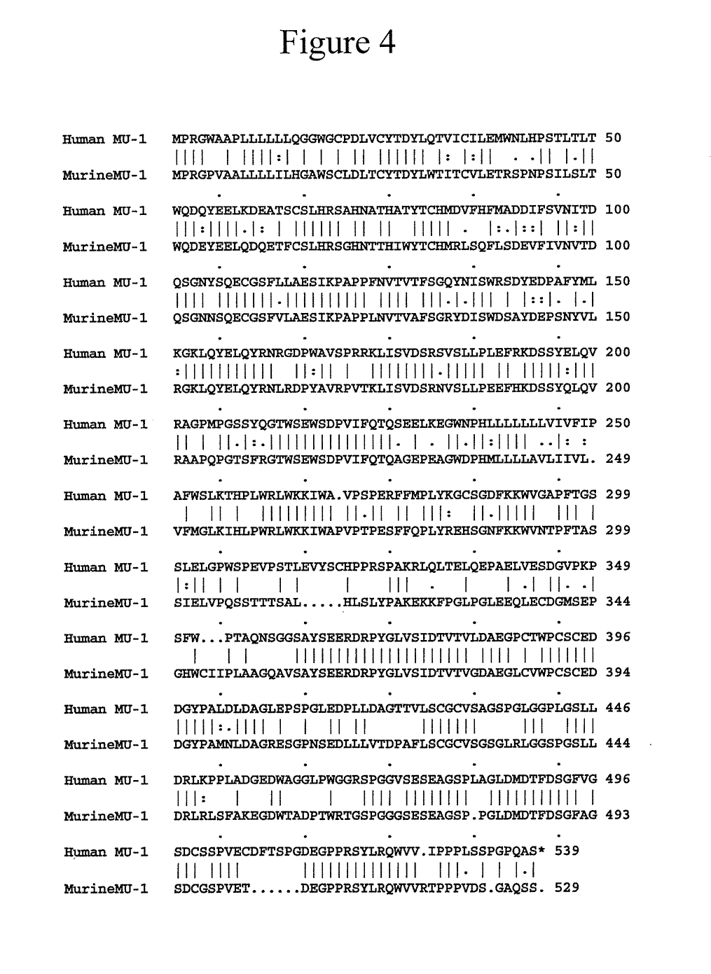

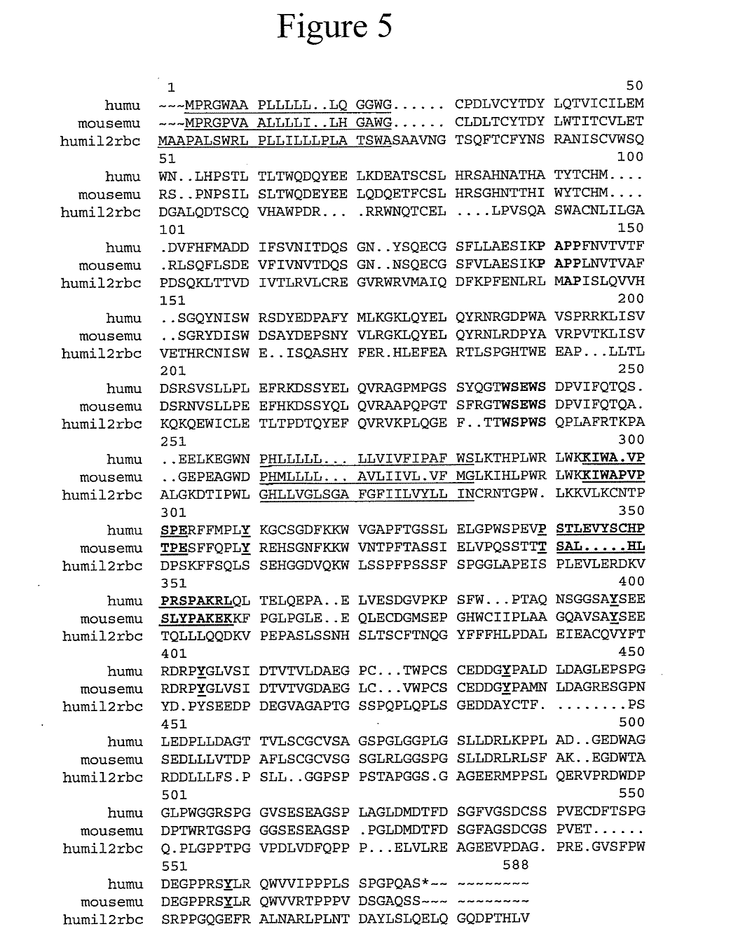

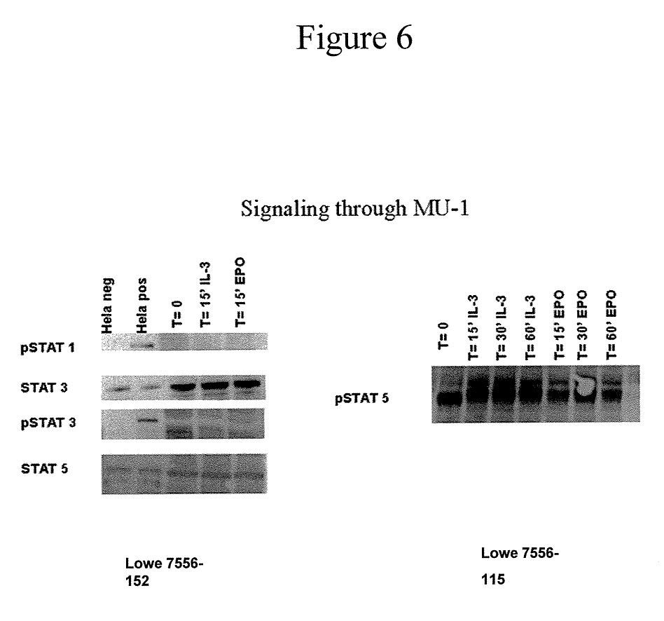

The GAP algorithm was used to compare the human and murine MU-1 amino acids. Human MU-1 was cloned using a 70-amino acid region of the human IL-5 receptor (SEQ ID NO:3) for searching a GenBank database, as well as primers for PCR (SEQ ID NOs:4 and 5), and hybridization oligonucleotides (SEQ ID NOs:6 and 7). A comparison of the murine and human predicted protein sequences is shown in FIG. 4. The amino acids were 65.267% identical using the GAP algorithm. The alignment was generated by BLOSUM62 amino acid substitution matrix (Henikoff and Henikoff (1992) Proc. Natl. Acad. Sci. U.S.A. 89: 10915-19). Gap parameters=Gap Weight: 8, Average Match=2.9 12, Length Weight=2, Average Mismatch=−2.003; Percent Similarity=69.466.

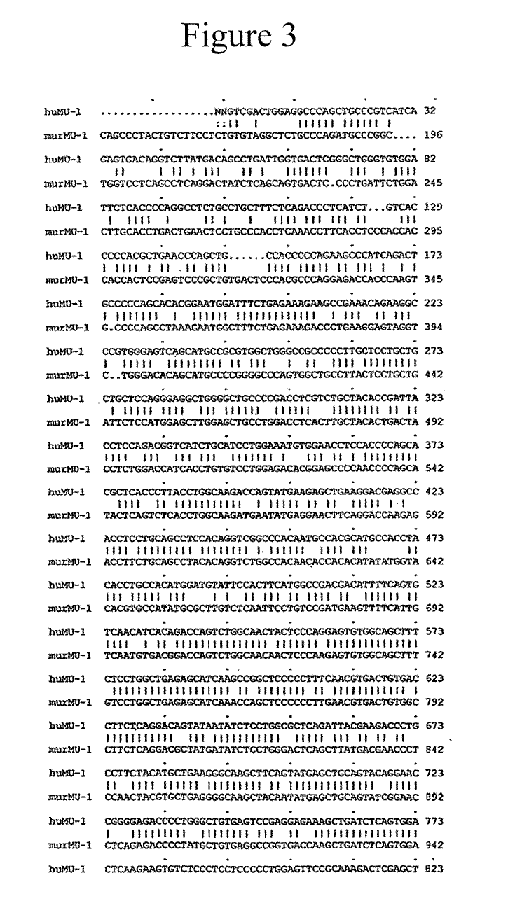

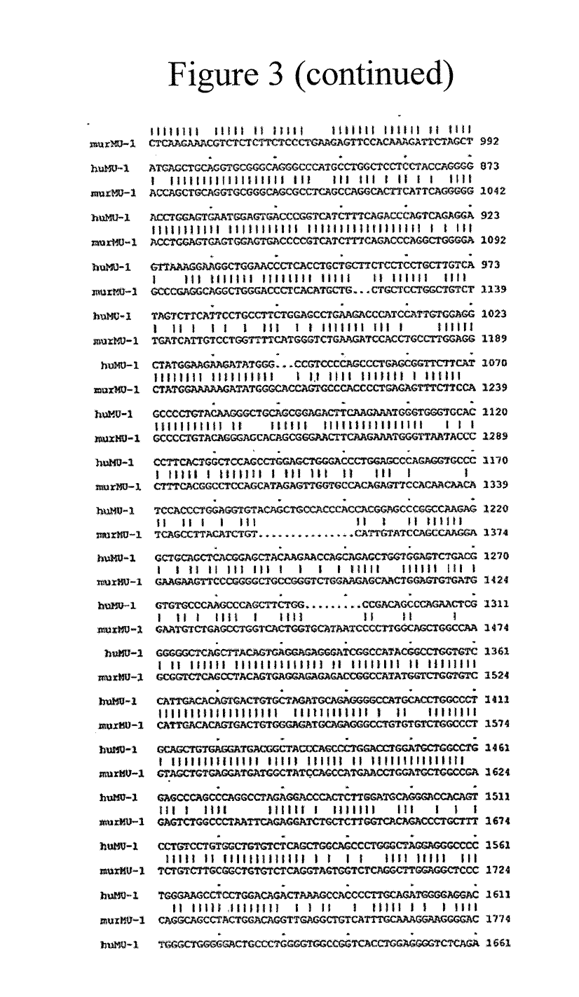

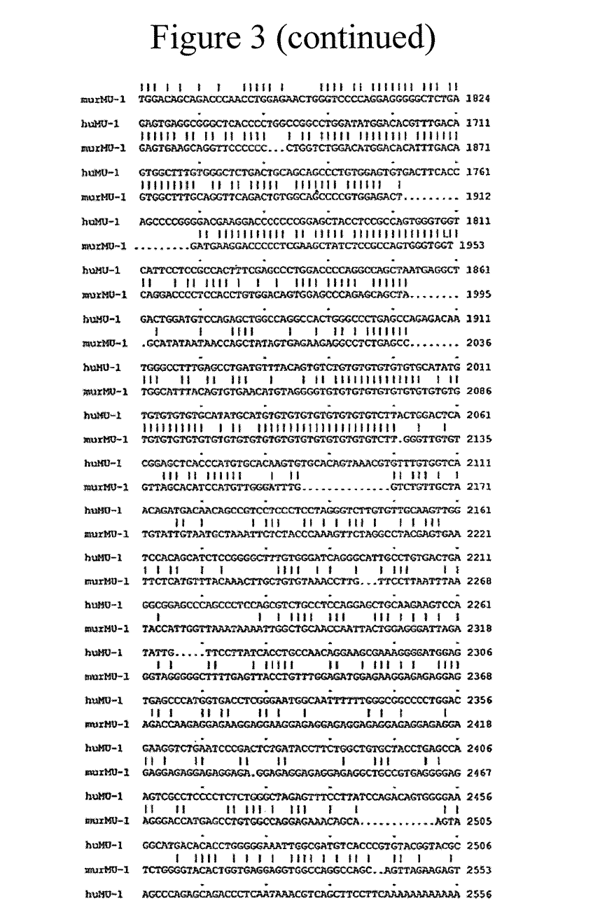

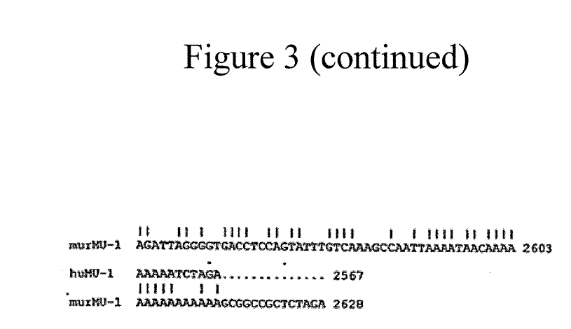

A comparison of the human and murine cDNA nucleotide sequences is shown in FIG. 3. The DNA sequences are 66.116% identical when aligned using the GAP algorithm. Gap Parameters: Gap Weight=50, Average Match 10.000, Length Weight=3, Average Mismatch=0.000, Percent Similarity=66.198.

Both human and mouse MU-1 proteins are members of the Type 1 cytokine receptor superfamily. Evaluation of the sequence of both murine and human MU-1 reveals the presence of potential Box-1 and Box-2 signaling motifs. Six tyrosine residues are present in the cytoplasmic domain and could also be important in signaling functions of MU-1. Comparison of the sequences of MU-1 with other members of the family suggested the presence of potential docking sites for STAT 5 and STAT 3.

### Example 3

### Determination of STAT Signaling Pathways Used by Human MU-1

BAF-3 cells were engineered to express a chimeric cytokine receptor consisting of the extracellular domain of the human EPO receptor and the intracellular domain of the MU-1 receptor. BAF-3 cells that expressed huEPORJMU-I(cyto) chimeric receptors proliferated in response to human soluble EPO. These cells were analyzed to determine which STAT molecules were phosphorylated in response to EPO signaling. Briefly, control unmodified parental BAF-3 cells and EPOR/MU chimeric BAF-3 cells were rested from IL-3 containing growth medium, and restimulated with either IL-3 or EPO for 0, 15, 30 and 60 minutes. The cells were pelleted and resuspended in ice-cold lysis buffer containing orthovanadate to preserve phosphorylated tyrosines. Equal amounts of cell lysate were electrophoresed by SDS-PAGE and blotted onto nitrocellulose membranes for western analysis. Duplicate blots were stained for phosphorylated and nonphosphorylated forms of STAT 1, 3, 5, and 6 by using antibodies specific for each form of the STAT molecule. HELA cells, nonactivated and activated with alpha-interferon, were used as positive controls.

These results indicated that under these specific conditions, signaling through MU-1 results in the phosphorylation of STAT 5 at all time points tested (T=0, T=15′, T=30′, T=60′). Treatment of controls or the chimeric BAF-3 cells with IL-3 resulted in phosphorylation of STAT 3, but not STAT 1 or 5.

### Example 4 Tissue Expression of Murine and Human MU-1

### Example 4.1

### Northern Analysis

Northern blots of polyA+ RNA from various tissues (Clonetech, Palo Alto, Calif.) were performed as recommended by the manufacturer. For the murine blots, a 262-nucleotide fragment corresponding to nucleotides 781-1043 of FIG. 1 and SEQ ID NO:9 was used for hybridization.

A single transcript of murine MU-1 was detected in adult murine spleen, lung, and heart tissues. The larger transcript observed in human tissues was not observed in mouse tissues.

Two transcripts of human MU-1 were detected in adult human lymphoid tissues, PBLs, thymus, spleen and lymph node, and in fetal lung.

### Example 4.2

### In Situ Hybridization

In situ hybridization studies were performed by Phylogency Inc. of Columbus, Ohio (according to the method of Lyons et al. (1990) J. Cell. Biol. 111:2427-36). Briefly, serial 5-7 micron paraffin sections were deparaffinized, fixed, digested with proteinase K, treated with tri-ethanolamine and dehydrated. cRNAs were prepared from linearized cDNA templates to generate antisense and sense probes. The cRNA transcripts were synthesized according to manufacturer's conditions (Ambion) and labeled with ³⁵S-UTP. Sections were hybridized overnight, washed under stringent conditions, and treated with RNase A and dipped in nuclear track emulsion and exposed for 2-3 weeks. Control sections were hybridized with sense probes to indicate the background level of the procedure. The murine probe consisted of a 186-bp fragment corresponding to nucleotides 860-1064 (SEQ ID NO:9). The human probe was a 23-bp PCR product generated from human MU-1 DNA.

Murine MU-1 expression was observed in the lymph nodes of the adult small intestine at germinal centers. Specialized lymph nodes and Peyers patches also exhibited murine MU-1 expression.

Human MU-1 expression was detected at germinal centers of the lymph nodules in the cortex. The medulla, which contains macrophages, was negative for human MU-1. In human spleen, human MU-1 expression was detected in the regions of white pulp but not red pulp.

### Example 5

### Expression of Human MU-1 in Cells and Cell Lines

RNase protection analysis was performed on resting and activated human T cells and the B cell lines, Raji and RPMI 8866, and the T cell line Jurkat. Human T cells were activated with anti-CD3 and anti-CD28. The cell lines were activated by phorbol ester and ionomycin. MU-1 riboprobe-producing plasmid was constructed by inserting a 23-bp PCR product (PCR was performed by using 5′ primer CACAAAGCTTCAGTATGAGCTGCAGTACAGGAACCGGGGA (SEQ ID NO:15) and 3′ primer CACAGGATCCCTTTAACTCCTCTGACTGGGTCTGAAAGAT (SEQ ID NO:16) into the BamHI and HindIII sites of pGEM3zf(−) (Promega, Madison, Wis.) vector). To make the riboprobe, the riboprobe-producing plasmid was linearized with HindIII. The resulting DNA was phenol/chloroform extracted and precipitated with ethanol. T7 RNA polymerase was used to make the riboprobe according to the protocol suggested by the vendor (PharMingen, San Diego, Calif.). The RNase protection assay was performed by using PharMingen's RIBOQUANT™ Multi-Probe Ribonuclease Protection Assay system. 2.0 μg of total RNA were included in each RPA reaction. After RNase digestion, the protected riboprobes were run on a QUICKPOINT™ rapid nucleic acid separation system (Novex, San Diego, Calif.). Gels were dried and exposed according to the suggestion of the vendor.

Human MU-1 RNA is upregulated in anti-CD3+ anti-CD28-stimulated human purified CD3+ cells when compared with unstimulated populations. MU-1 is also upregulated upon restimulation in Th1 and Th2-skewed T cell populations. The B cell lines, RPMI 8866 and Raji, constitutively express MU-1 while the Jurkat T cell line does not.

### Example 6

### Binding of Human MU-1 to Known Cytokines

Both human and murine Ig fusion proteins were constructed and immobilized on Biacore chips in an effort to identify the ligand for MU-1. A variety of cell culture conditioned media as well as a panel of known cytokines were evaluated for binding to MU-1. Some cytokines were also tested in combination with other receptor chains in the family to consider the possibility that MU-1 may require a second receptor chain for ligand binding. The following cytokines were tested and found to be negative for MU-1 binding: mIL-2, hIL-2, hIL-15, mIL-7, TSLP, TSLP+IL-7, TSLP+IL-7R, TSLP+IL-7g, TSLP+IL-2, TSLP+IL-2+IL-2Rbeta, IL2-Rbeta, IL-2Rgamma, IL-7R, IL-2+IL-2Rbeta, IL-2+IL-2Rgamma, IL-15+IL-2Rbeta, IL-15+IL-2Rgamma, IL-7+IL-2Rgamma, IL-2+IL-7R, IL-15+IL-7R, IL-7+IL-7R. Known receptors have been immobilized as well and tested for MUFc binding with negative results. IL-15 will bind to IL-2Rb but not IL-2Rg or MUFc.

### Example 7

### Inhibition of IL-21/IL-21R Activity Ameliorates the Severity of Symptoms in Collagen-Induced Arthritis (CIA) Mice

This example shows that IL-21R antagonists, e.g., IL-21R-Ig fusion proteins (murine IL-21RFc protein or “muIL-21RFc”) or anti-IL-21R antibodies, ameliorate symptoms in a CIA murine model.

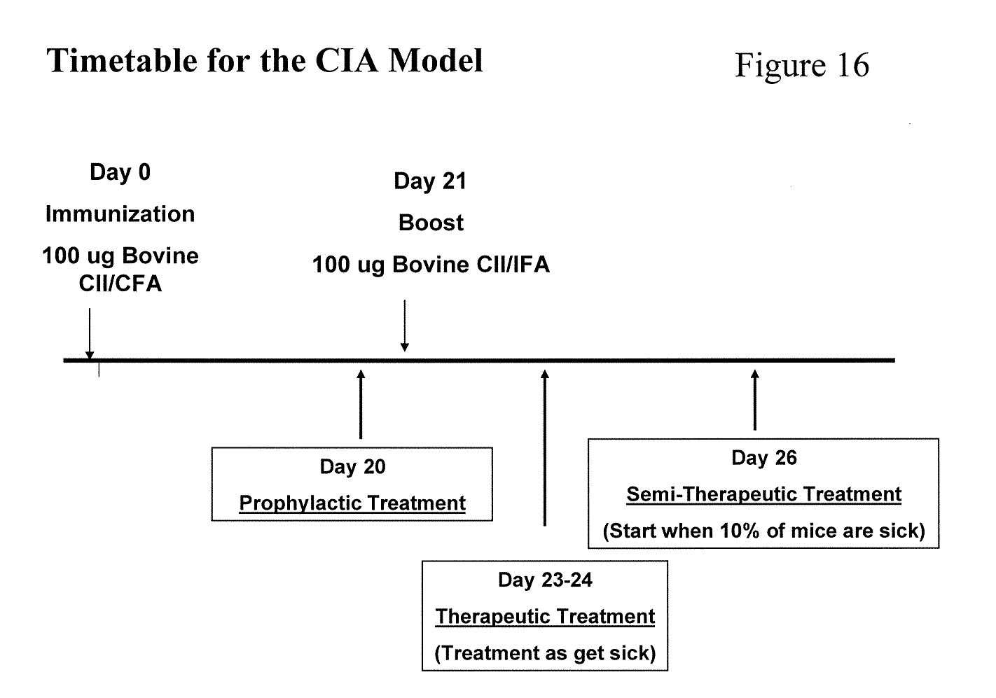

Male DBA/1 (Jackson Laboratories, Bar Harbor, Me.) mice were used for all experiments. Arthritis was induced with the use of bovine collagen type II (Chondrex, Redmond, Wash.). Bovine collagen type II (Chondrex) was dissolved in 0.1 M acetic acid and emulsified in an equal volume of complete Freund's adjuvant (Sigma) containing 1 mg/ml Mycobacterium tuberculosis (strain H37RA). 100 μg of bovine collagen was injected subcutaneously in the base of the tail on day 0. On day 21, mice were injected subcutaneously, in the base of the tail, with a solution containing 100 μg of bovine collagen in 0.1 M acetic acid that had been mixed with an equal volume of incomplete Freund's adjuvant (Sigma). Naive animals received the same sets of injections, minus collagen. The dosing protocol is shown schematically in FIG. 16. MuIL-21RFc was administered prophylactically or therapeutically to DBA mice. In the therapeutic regimen, treatment was initiated if disease was observed for two consecutive days in a mouse.

Mice were monitored at least three times a week for disease progression. Individual limbs were assigned a clinical score based on the index: 0=normal, no swelling; 1=visible erythema accompanied by 1-2 swollen digit, or mild swelling in ankle; 2=pronounced erythema, characterized by mild to moderate paw swelling and/or two swollen digits; 3=extensive swelling of the entire paw, i.e., extending into ankle or wrist joint; 4=resolution of swelling, ankylosis of the paw; difficulty in use of limb or joint rigidity. Thus, the sum of all limb scores for any given mouse yielded a maximum total body score of 16.

At various stages of disease, animals were euthanized, tissues were harvested and paws were fixed in 10% formalin for histology or 4% paraformaldehyde, pH 7.47, decalcified in 20% EDTA (pH 8.0) and embedded in paraffin for in situ hybridization. Using light microscopy the paws were scored on a 5-grade scoring method (0-4) to characterize the intensity and extent of arthritis. Inflammatory infiltrates were used for scoring in addition to other changes related to the inflammation, such as pannus formation, fibrous of the synovial membrane, articular cartilage erosion and/or subchondral bone destruction. Histology grades were determined using readings of individual paws: NAD=0 or nothing abnormal discovered; 1=slight to moderate; 2=mild to moderate; 3=marked; and 4=massive.

A reduction in the severity of the symptoms was observed after prophylactic treatment of CIA mice using muIL-21RFc (100 μg or 200 μg) administered intraperitoneally (IP) every other day starting one day before the collagen boost (data not shown).

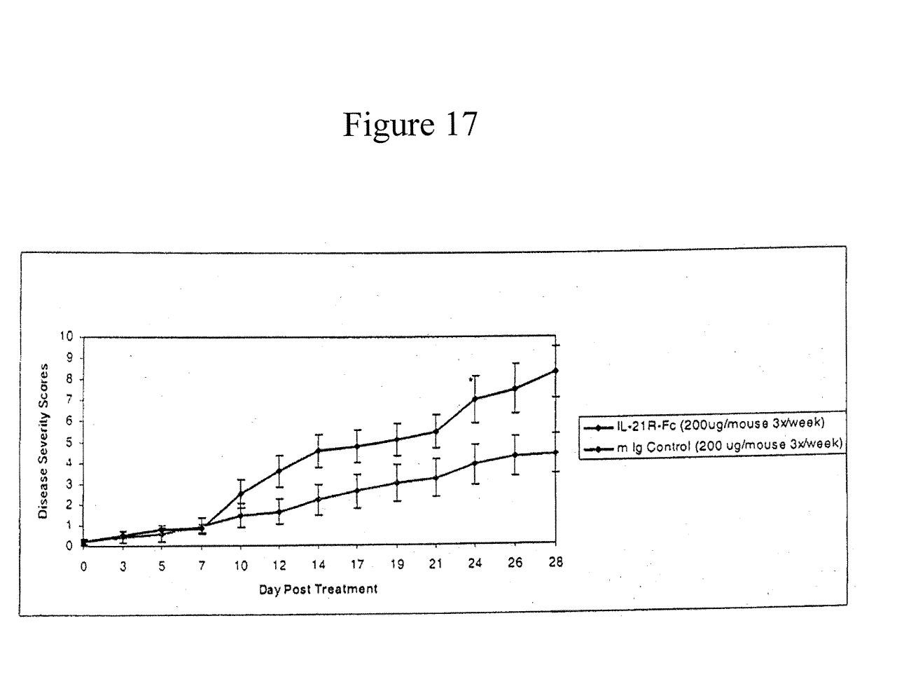

The effects of muIL-21RFc (200 μg/mouse 3×/week) on a semi-therapeutic CIA mouse as a function of day post-treatment are shown in FIG. 17. Mouse Ig (200 μg/mouse 3×/week) was used as a control. A reduction in the severity score is shown starting from day 7 post-treatment.

These experiments demonstrate that administration of an IL-21R antagonist, e.g., IL-21R-Fc fusion proteins, to CIA mice either prophylactically or semi-therapeutically significantly ameliorated arthritic symptoms.

### Example 8

### In Situ Hybridization of IL-21R Transcripts

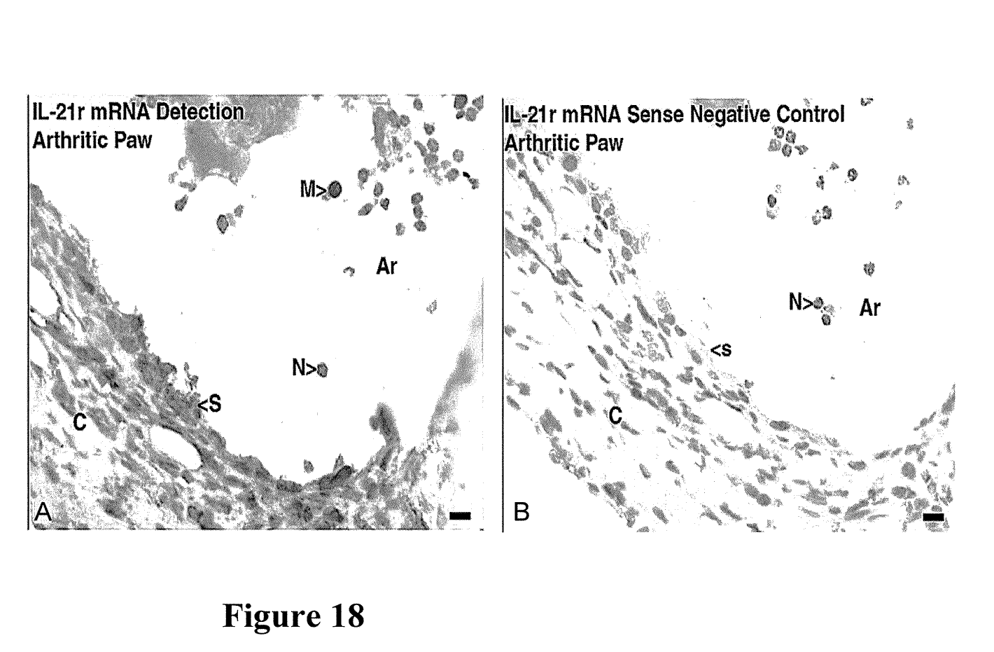

The expression of IL-21R mRNA in arthritic paws of mice with CIA was determined. Anti-sense murine IL-21R riboprobes were used (FIG. 18A); sense probes were used as negative controls (FIG. 18B). Digoxygenik-labeled probes were prepared with the use of a DIG RNA labeling mix (Roche Diagnostics, Mannheim, Germany), as described by the manufacturer. Expression of IL-21 receptor mRNA was detected in macrophages, neutrophils, fibroblasts, a subpopulation of lymphocytes, synoviocytes and epidermis (FIG. 18A). Decreased staining was seen in the control paws or with sense probes (FIG. 18B). mIL-21R mRNA positive cells were: neutrophils (N), and macrophages (M). In situ hybridization shows enhanced expression of IL-21R in the paws of arthritic mice.

### Example 9

### Inhibition of IL-21/IL-21R Activity Ameliorates the Severity of IBD-Like Symptoms in the HLA-B27 Rat Model

This example shows that IL-21R antagonists, e.g., IL-21R-Ig fusion proteins (murine IL-21RFc protein or “muIL-21RFc”) or anti-IL-21R antibodies, ameliorate IBD-like symptoms in HLA-B27 rat model.

A murine IL-21Receptor-Fc fusion polypeptide (MuIL-21RFc) was generated as described herein and was evaluated for its ability to alleviate inflammation of the bowel in the HLA-B27 rat model. The HLA-B27 rat model has been extensively used to evaluate IBD therapies because the bowel inflammation observed in the model shares several clinical, histological, and immunological features with IBD in humans (reviewed in, e.g., Elson et al. (1995) Gastroenterology, 109:1344-67; Blanchard et al. (2001) European Cytokine Network 12:111-18; Kim et al. (1999) Arch. Pharm. Res. 22:354-60). For example, the HLA-B27 rat overexpresses human major histocompatibility complex I allele B27 and B2-microglobulin gene products. Such gene products are associated with the development of chronic inflammatory diseases, such as IBD.

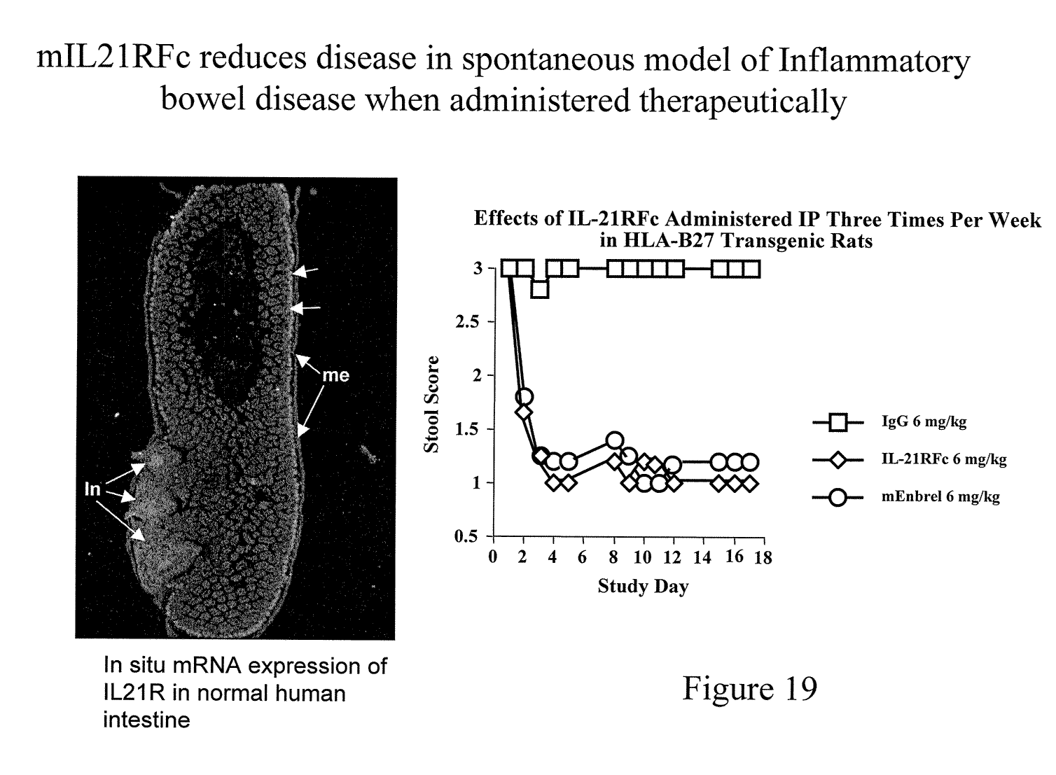

Rats utilized in the study had developed chronic inflammation of the gastrointestinal tract (GI) as evidenced by clinical signs of persistent diarrhea. Stools were assigned a clinical score (0-3) based on the index: 0=normal with formed stool pellets; 1=soft, with formed stool pellets; 2=loose, no formation of stool pellets; and 3=watery diarrhea (see FIG. 19). The rats were monitored for 18 days during which stools were evaluated for disease progression. A clinical score of 3 is indicative of persistent diarrhea (shown as IgG control). MuIL-21RFc was administered (6 mg/kg IP, 3× week) to five HLA-B27 transgenic rats/group for a period of 18 days. Another group was given 6 mg/ml mEnbrel (soluble TNF-receptor Fc fusion), a positive control. A third group, consisting of an equal number of mice, was administered IgG as a control in the same manner and dosage.

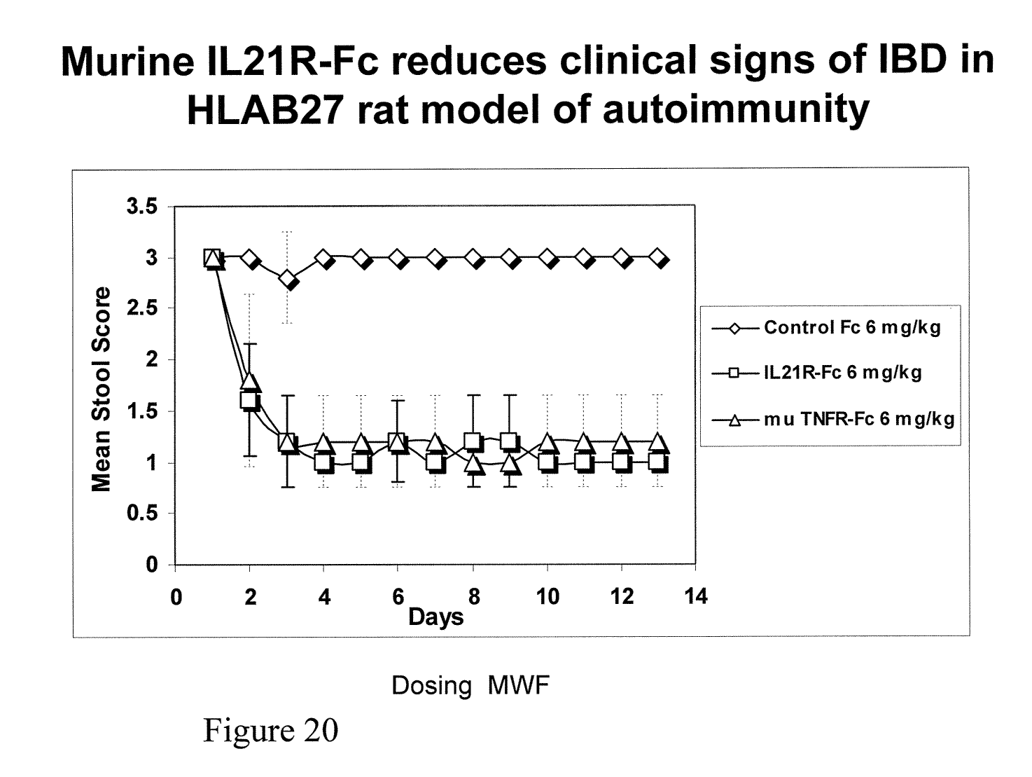

A marked reduction in the clinical score was detected in the groups treated with MuIL-21RFc and mEnbrel, compared to the IgG control (see FIGS. 19 and 20). Administration of MuIL-21RFc showed an efficacy similar to mEnbrel in ameliorating IBD-like symptoms. Results from this study demonstrate that the administration of MuIL-21RFc decreases bowel inflammation with similar efficacy as mEnbrel in a HLA-B27 rat model relative to rats administered control IgG (see FIGS. 19 and 20).

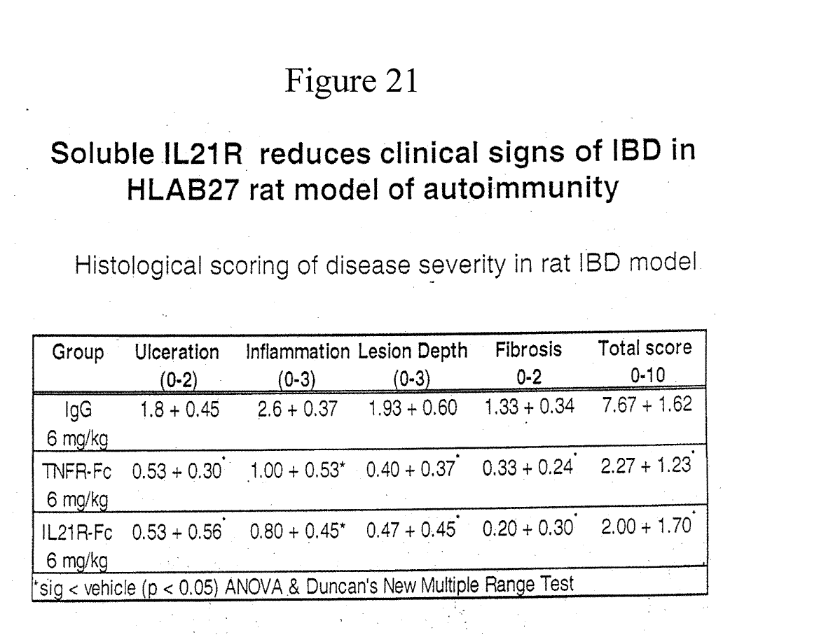

The alleviation of symptoms expressed in terms of improved stool score was confirmed by histological analysis. Rats treated with MuIL21RFc scored significantly lower disease severity than those treated with control, IgG, in regards to ulceration, inflammation, lesions depth, and fibrosis (see FIG. 21). The histological analysis was assigned a clinical score from 0-2 or 0-3, as indicated, where a higher score is indicative of increased severity in the rat IBD model. A significant decrease of inflammation in the bowel was detected in all categories examined in groups treated with MuIL-21RFc and mEnbrel relative to control. MuIL-21RFc showed a similar efficacy as mEnbrel in ameliorating the histological signs of disease severity. To support an extension of the results shown above to humans, FIG. 19 (right side panel) shows in situ hybridization of MU-1 mRNA in the lymphocytes and lymph nodes of the normal human intestine, indicating expression of MU-1 mRNA in the organ relevant to the disease.

### Example 10

### Inhibition of IL-21/IL-21R Activity Delays Allogeneic Skin Graft Rejection in Mice

This example shows that IL-21R antagonists, e.g., IL-21R-Ig fusion proteins (murine IL-21RFc protein or “muIL-21RFc”) or anti-IL-21R antibodies, delay allogeneic skin graft rejection in mice, and thus prolongs graft survival.

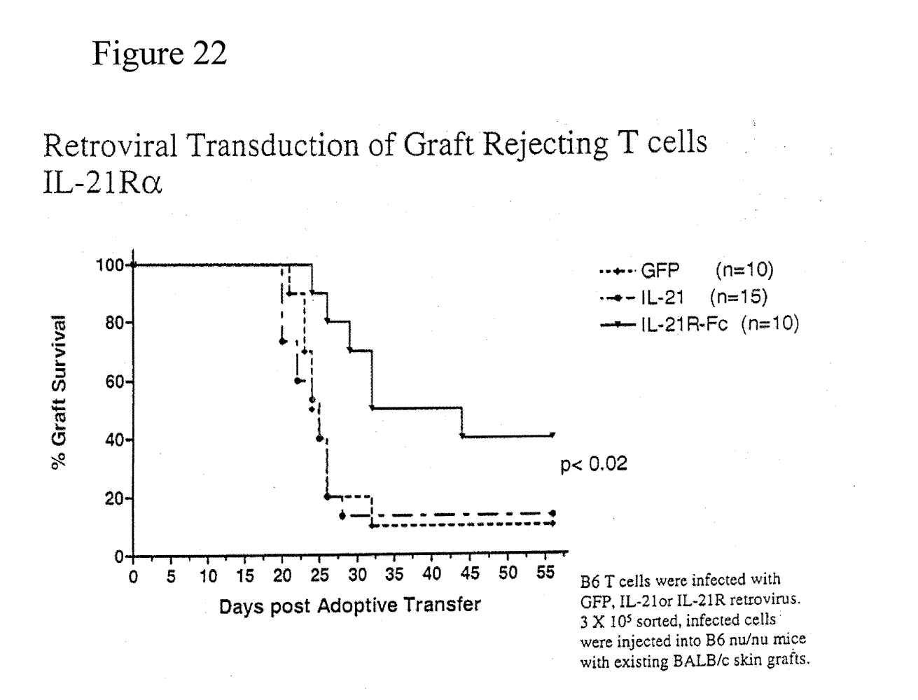

Administration of MuIL-21RFc was shown to delay allogeneic skin graft rejection in mice injected with retrovirally transduced T cells. FIG. 22 depicts a graph showing the percentage of graft survival relative to days post-adoptive transfer. In this model, nude mice show healed allogeneic skin grafts because the mice have no detectable T cells. When activated B6 T cells that had been retrovirally engineered to secrete control GFP or IL-21 were injected into the nude mice, grafts were rejected (see FIG. 22). If the T cells were engineered to secrete MuIL-21RFc (which is expected to neutralize IL-21-made by these cells), the grafts survived for longer time intervals as shown in FIG. 22 (indicated by the IL-21R-Fc compared to the GFP and IL-21 controls). Ten mice were used for the GFP and MuIL-21RFc, respectively; fifteen mice were used for the IL-21 controls. These results demonstrate a role for IL-21R antagonists in prolonging graft survival.

### Example 11

### Inhibition of IL-21/IL-21R Activity Reduces Disease Symptoms in a CD45RB^{hi} Adoptive Transfer Model

This example shows that IL-21R antagonists, e.g., IL-21R-Ig fusion proteins (murine IL-21RFc protein or “muIL-21RFc”) or anti-IL-21R antibodies, ameliorate symptoms in a mouse model of psoriasis and inflammatory bowel disease (IBD).

Transfer of CD45RB^{hi} CD4⁺ naïve T cells into severe combined immunodeficient (SCID) mice results in colitis and/or skin lesions resembling psoriasis, depending upon cage housing conditions. BALBc CD45RB^{hi} CD4⁺ T cells (naive population) were sorted from spleen cells first by negative selection on columns for CD4⁺ T cells and then further sorted by flow cytometry, selecting for high CD45 expression. 4×10⁵ cells of this population were transferred into female C.B-17 SCID mice, and the mice were scored for several weeks for clinical signs of psoriasis and IBD. Mice housed under static cage conditions develop inflammatory bowel disease; mice housed under regular conditions with air flow changes also develop psoriasis. Mice were scored for psoriasis on a scale from 1-6:1=mild, moderate erythema (usually eyelids and ears), <2% of body; 2=mild scaling and moderate to severe erythema (usually ear and face), 2-10% of body; 3=severe erythema and scaling (ear face and trunk), 10-20% of body; 4=very severe erythema throughout body, 20-40% of body; 5=very severe erythema throughout body, 40-60% of body; 6=very severe erythema throughout body, 60-100% of body. Mice were scored for IBD by weight loss and stool score: 0=normal; 1=soft; 2=diarrhea; 3=diarrhea containing blood and mucus.

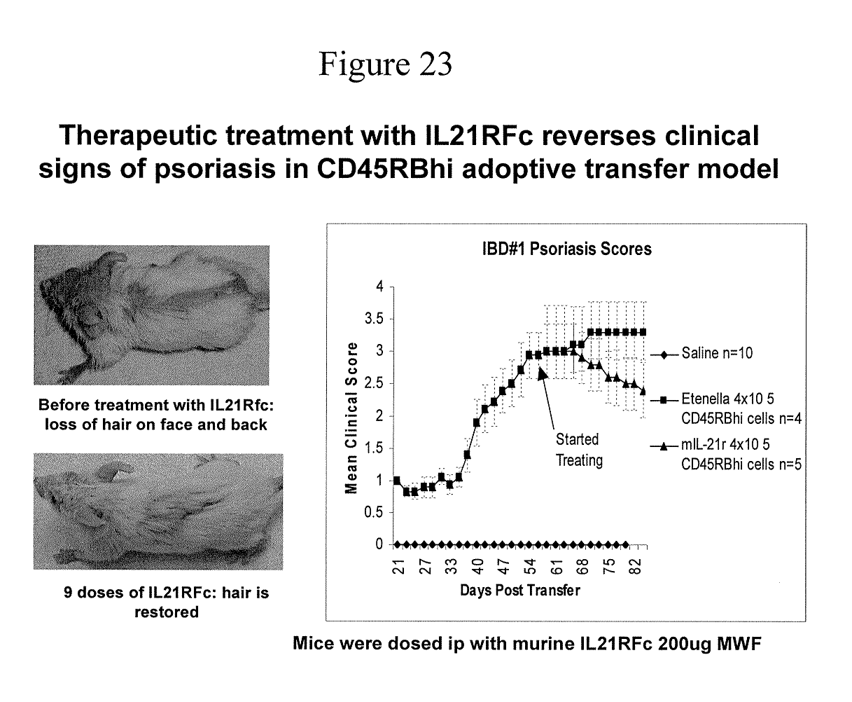

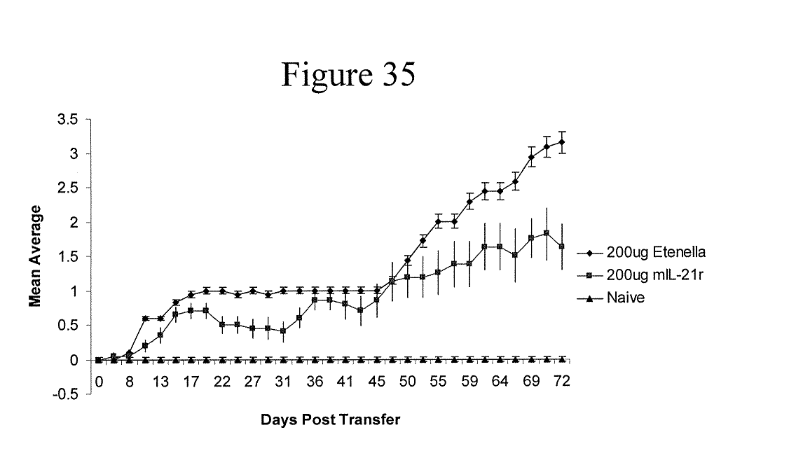

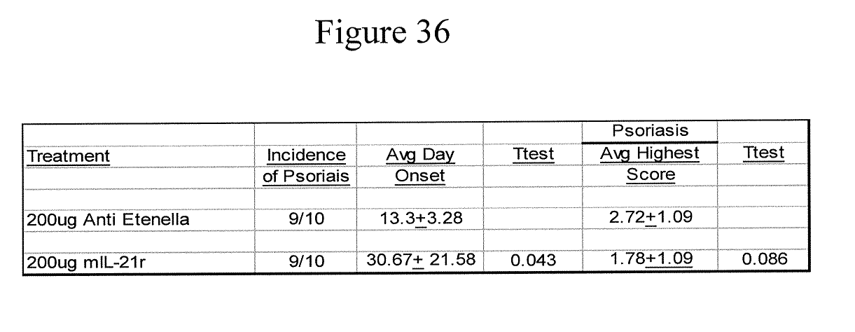

Treatment using muIL-21RFc was effective in ameliorating psoriasis-like symptoms. In mice that developed skin inflammation, treatment by intraperitoneal injection with 200 μg muIL-21RFc 3× per week beginning eight weeks after CD45RB^{hi} cell transfer resulted in reduced erythema, scaling and hair loss when compared to control mice treated with anti-E. tenella Ig (FIG. 23). Treatment of CD45RB^{hi} recipient mice with 200 μg muIL-21RFc 3× per week at the time of cell transfer resulted in delayed onset of psoriasis and less severe clinical disease compared to controls over the course of the experiment (FIG. 35). The results of the experiment are summarized in FIG. 36.

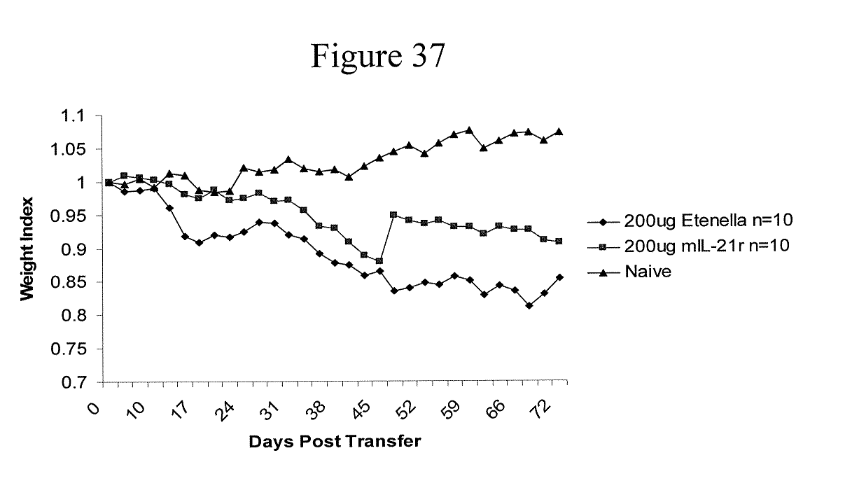

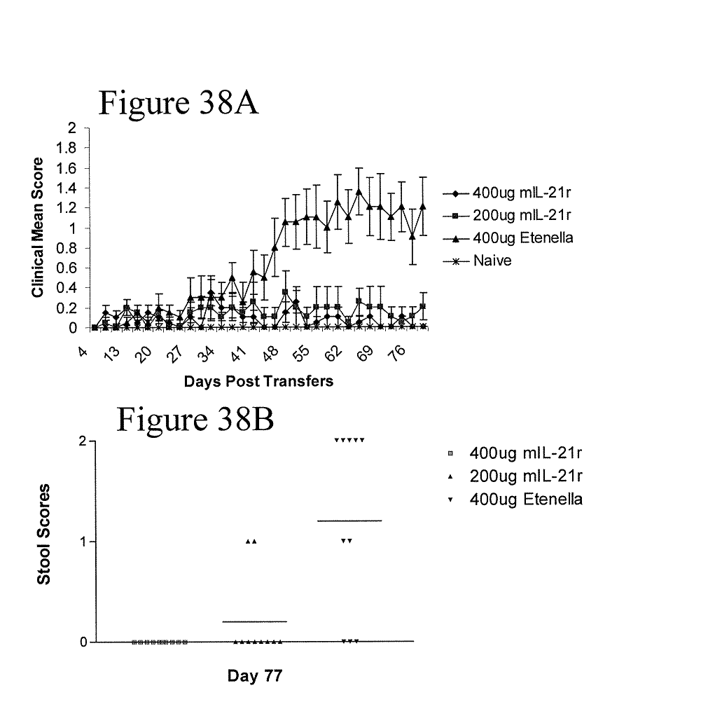

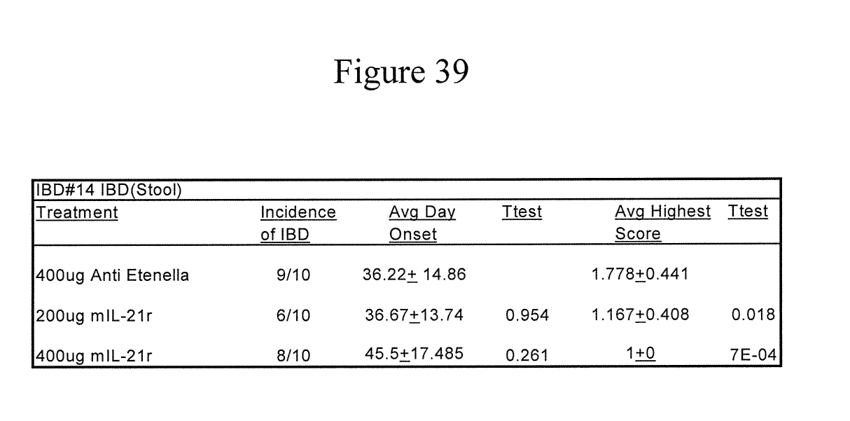

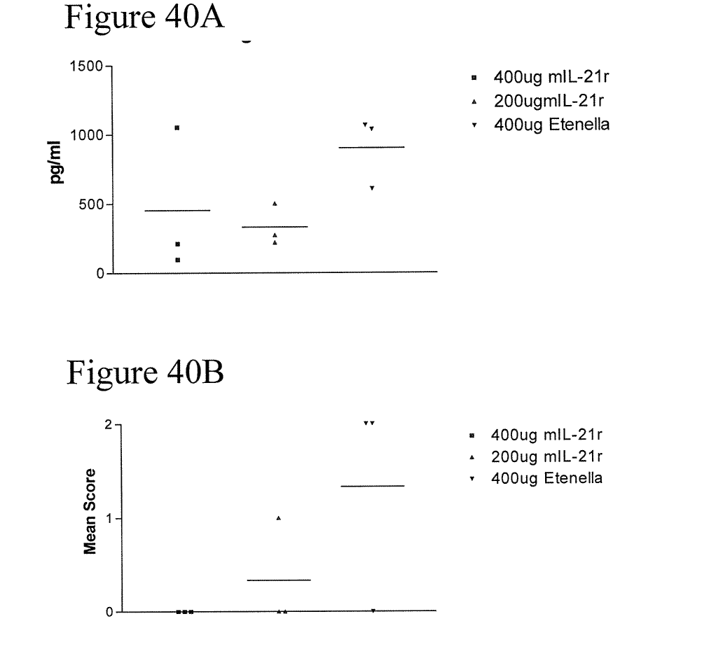

Treatment using muIL-21RFc was also effective in ameliorating inflammatory bowel symptoms. Treatment of CD45RB^{hi} recipient mice with 200 μg or 400 μg muIL-21RFc three times per week at the time of cell transfer resulted in a significant reduction of clinical signs of colitis as measured by body weight loss (FIG. 37) and stool score (FIG. 38) when compared with Ig control-treated mice. The results are summarized in FIG. 39. Macroscopic evaluation of colons from control-treated CD45RB^{hi} recipients showed severe thickening and swelling which was almost completely suppressed in mice treated with muIL-21RFc. Microscopically, control-treated mice also exhibited a greater degree of epithelial hyperplasia and leukocyte infiltration in the lamina propria/submucosa when compared with muIL-21RFc-treated mice. Additionally, serum cytokines were measured from control-treated mice and muIL-21RFc-treated mice. Of several cytokines measured, only gamma interferon (IFN-γ) was detectable in the serum. Treatment with muIL-21RFc at 200 μg or 400 μg doses resulted in significantly reduced serum levels of IFN-γ when compared with Ig control-treated mice (FIG. 40). IFN-γ can be used as a biomarker for IL-21R antagonist efficacy in IBD.

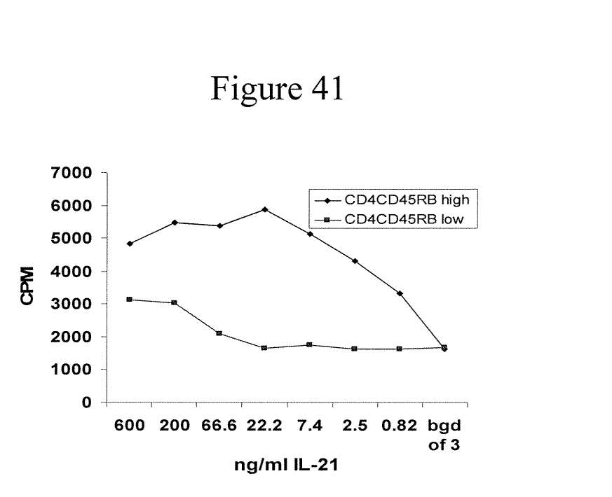

CD45RB^{hi} (naive) and CD45RB^{lo} (memory) subsets were tested by a proliferation assay for their response to IL21. In the IBD transfer model, only the naive cells cause disease, and disease can be suppressed by the addition of the memory population. In this assay, purified populations were stimulated with plate-bound anti-CD3 and tested for ³H-thymidine incorporation in response to IL-21. The naïve population showed a significantly increased response to IL-21 compared to the memory population (FIG. 41). This suggests that IL-21 is an important cytokine for the expansion of this population in vivo.

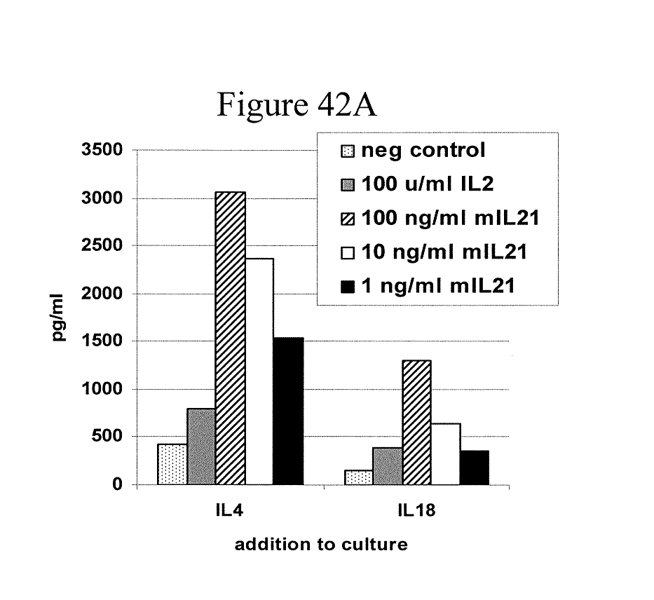

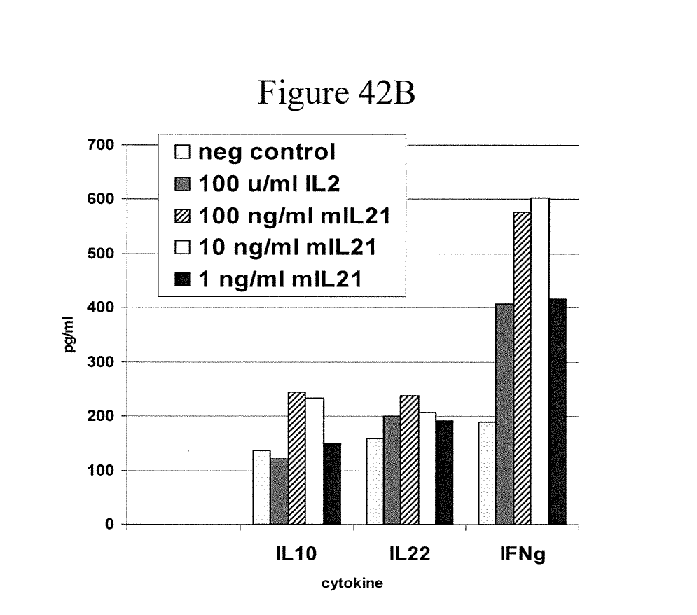

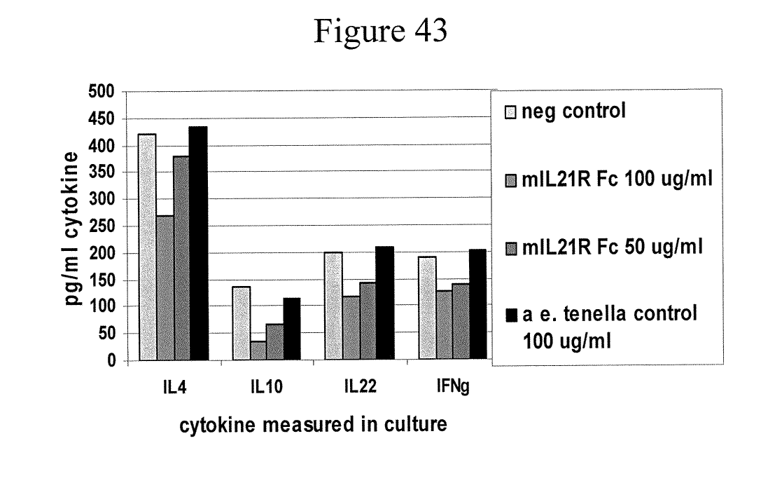

Addition of IL-21 to activated CD4⁺ CD45RB^{hi} cells in culture induced the secretion of multiple cytokines. Anti-CD3-stimulated CD45RB^{hi} CD4⁺ T cells were treated with 100 units/ml IL-2 or 1 ng/ml, 10 ng/ml or 100 ng/ml IL-21. In response to IL-21, CD45RB^{hi} cells secreted increased levels of IL-2, IL-4, IL-10, IL-17, IL-18, IL-22, IFN-γ and TNFα (FIG. 42). Blockade of endogenous IL-21 by addition of 50 μg/ml or 100 μg/ml muIL-21RFc resulted in decreased levels of cytokines in these cultures compared to cultures treated with an Ig control (FIG. 43).

Taken together, these results indicate that IL-21 is a potent potential player in the inflammatory responses in this model and that IL-21R antagonists can be of therapeutic benefit in Th1-mediated diseases such as Crohn's and psoriasis.

### Example 12

### Mice Lacking IL-21R Show a Reduction in Antigen-Induced Airway Inflammation

This example shows that transgenic knockout mice lacking the IL-21 receptor (IL-21R −/−) have a significantly reduced response to antigen-induced airway inflammation and airway hyperresponsiveness.

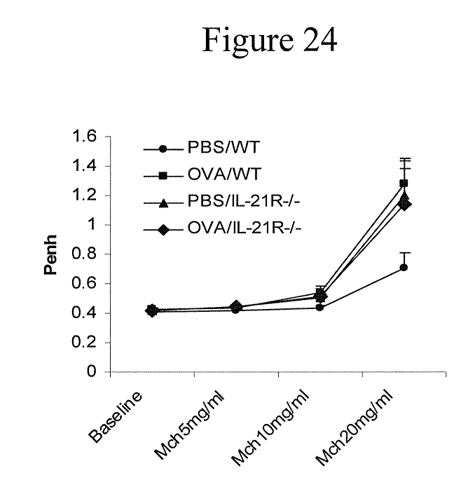

IL-21R −/− and wild type (WT +/+) C57BL/6 mice (8-12 weeks old) were immunized by intraperitoneal injection of 20 μg OVA emulsified in 2.25 mg alum (Alum Inject; Pierce) on days 0 and 14. On days 26, 27 and 28, the airways were challenged with an aerosol of 5% OVA in PBS for 30 min. Forty-eight hours after the last OVA challenge, animals were assessed for changes in lung resistance and dynamic compliance to aerosolized methacholine. OVA sensitization and challenge resulted in a significant increase in airway hyperresponsiveness after aerosolization of methacholine in WT +/+ mice when compared with OVA-sensitized PBS-challenged WT +/+ mice (FIG. 24). However, there was no difference of airway hyperresponsiveness in OVA-sensitized/OVA-challenged IL-21R −/−mice to aerosolized methacholine over the entire dose range compared to OVA-sensitized/OVA-challenged WT +/+ mice (FIG. 24).

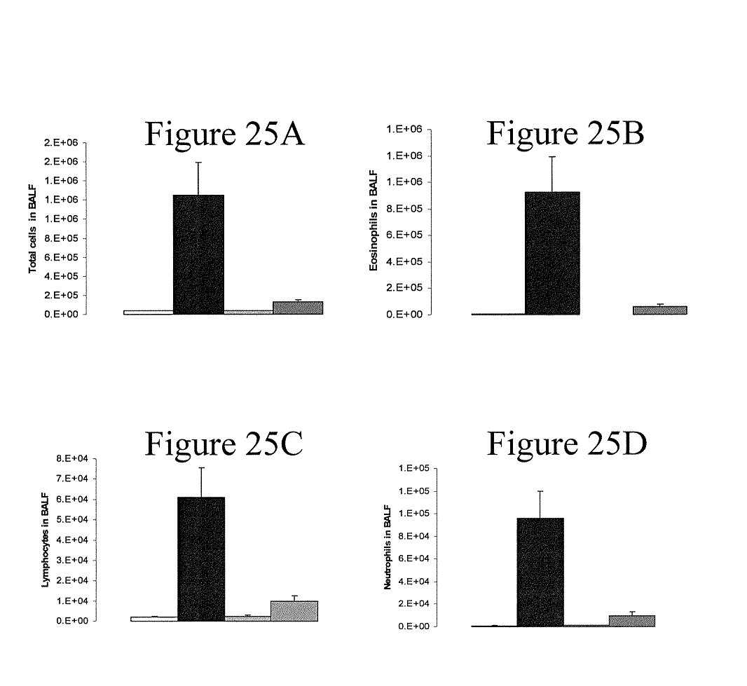

Animals were then sacrificed and blood and bronchoalveolar lavage fluid (BALF) collected for analysis of pulmonary inflammation, cytokine levels and total and anti-OVA IgE titers. BALF was collected by bronchoalveolar lavage with 3×0.7 ml of PBS. Total BALF cell numbers were increased approximately 36 fold after OVA challenge in WT +/+ mice, compared with PBS-challenged controls in contrast to a 3-fold increase over PBS-challenged controls in IL-21R −/− animals (FIG. 25A). Furthermore, total cell numbers within the BALF of OVA-sensitized/OVA-challenged IL-21R −/− mice were significantly lower than those observed in OVA-sensitized/OVA-challenged WT +/+ animals. There was no difference in BALF total cell numbers in OVA-sensitized/PBS-challenged IL-21R −/− and WT +/+ mice (FIG. 25A). OVA challenge resulted in a significant increase in BALF eosinophils in both WT +/+ and IL-21R −/− mice, compared to identically sensitized but PBS-challenged controls. Absolute numbers of BALF eosinophils were significantly attenuated in IL-21 −/− animals compared to those observed in OVA-sensitized/OVA-challenged WT +/+ animals (FIG. 25B). Deletion of IL-21R also significantly attenuated the increases in numbers of BALF lymphocytes (FIG. 25C) and neutrophils (FIG. 25D) after OVA challenge.

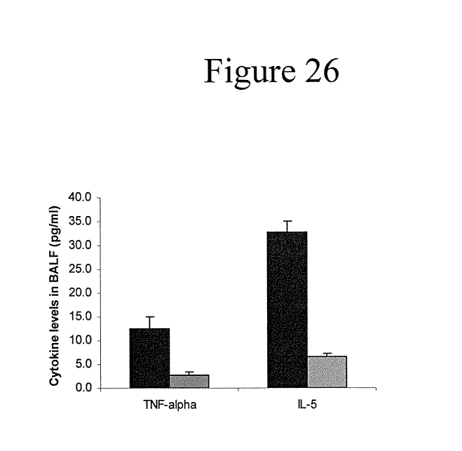

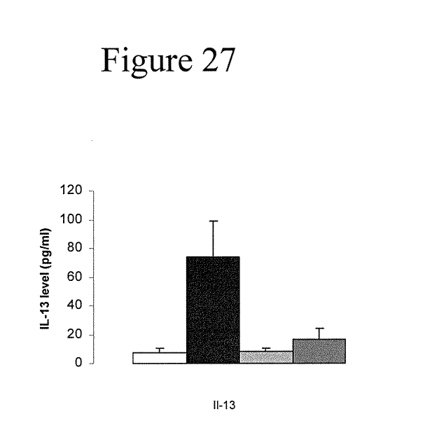

Levels of IL-5, IL-13 and TNFα within the BALF increased significantly in OVA-sensitized/challenged WT +/+ mice compared with PBS-challenged controls (FIGS. 26 and 27). In contrast, OVA-sensitization and challenge induced a very modest increase in the levels of these cytokines in the BALF of IL-21R −/− mice as compared with PBS-challenged controls and levels were significantly lower than those observed in OVA-sensitized/OVA-challenged WT animals (FIGS. 26 and 27). TNFα and IL-5 levels in BALF were quantified using a cytometric bead array kit (Mouse Th1/Th2 Cytokine CBA, BD Biosciences, San Diego, Calif.). IL-13 levels in BALF were quantified by ELISA.

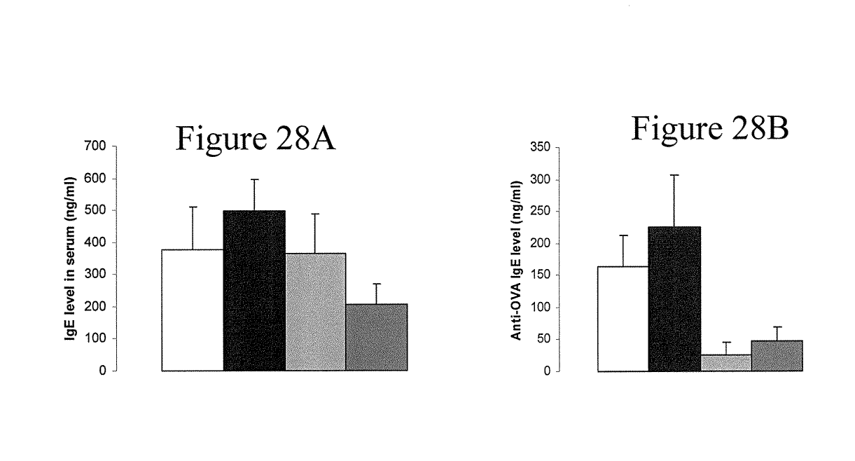

As shown in FIGS. 28A-B, serum total IgE and anti-OVA IgE levels after OVA sensitization/OVA challenge in IL-21R −/− were much lower compared with identically treated WT+/+ mice. However, there was no significant difference in the IL-21R −/− and WT +/+ mice when either total or OVA-specific IgE levels were compared after PBS challenge.

These results suggest that inhibition of IL-21-mediated responses can provide therapeutic value in the treatment of allergy and asthma.

### Example 13

### Inhibition of IL-21/IL-21R Activity Ameliorates the Severity of Symptoms in an MRL-FAS^{lpr} Lupus Model

This example shows that IL-21R antagonists, e.g., IL-21R-Ig fusion proteins (murine IL-21RFc protein or “muIL-21RFc”) or anti-IL-21R antibodies, ameliorate systemic lupus erythematosus (SLE)-like symptoms in an MRL-Fas^{lpr} mouse model.

Male MRL-Fas^{lpr} mice were used for all experiments. These mice present multiple symptoms similar to human SLE, including DNA autoantibodies, destruction of multiple tissues, and immune complex glomerulonephritis. 400 μg MuIL-21RFc or an isotype control was injected intraperitoneally three times per week beginning at 10 weeks of age, and the mice were analyzed weekly for disease progression. At 15 weeks, mice were sacrificed for further analysis. Each treatment group contained 10 mice.

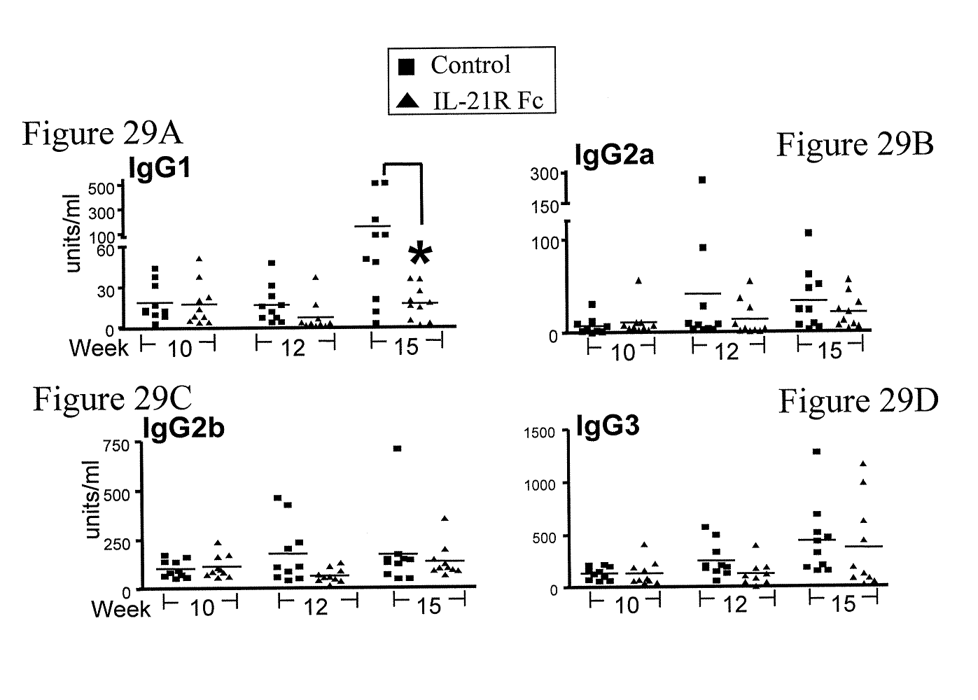

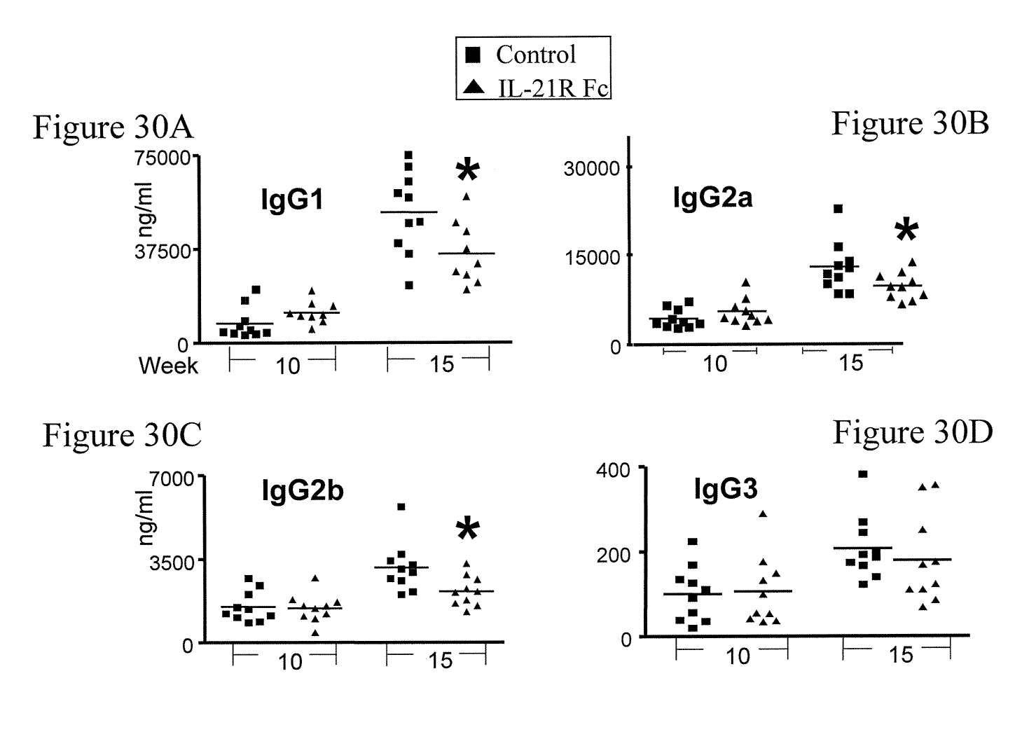

MuIL-21RFc treatment significantly reduced the levels of circulating anti-dsDNA autoantibodies (FIG. 29) and serum total IgG (FIG. 30) in MRL-Fas^{lpr} mice, as measured by ELISA. Briefly, for measurement of antidsDNA autoantibodies, dsDNA was coated on a titer plate, serum antibodies were added, and antibodies were detected using an anti-mouse secondary antibody. For measurement of total IgG, serum was adhered to a titer plate, followed by detection using an anti-mouse secondary antibody.

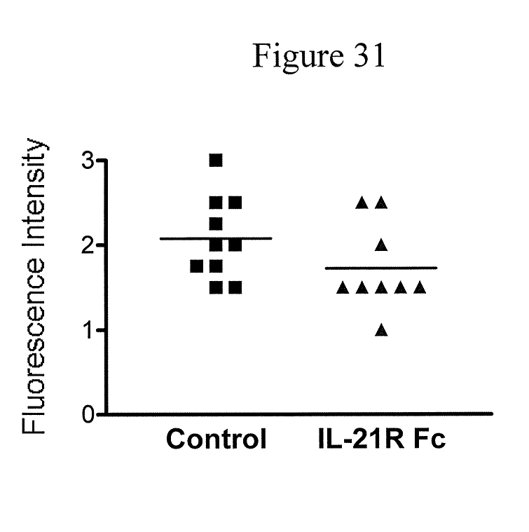

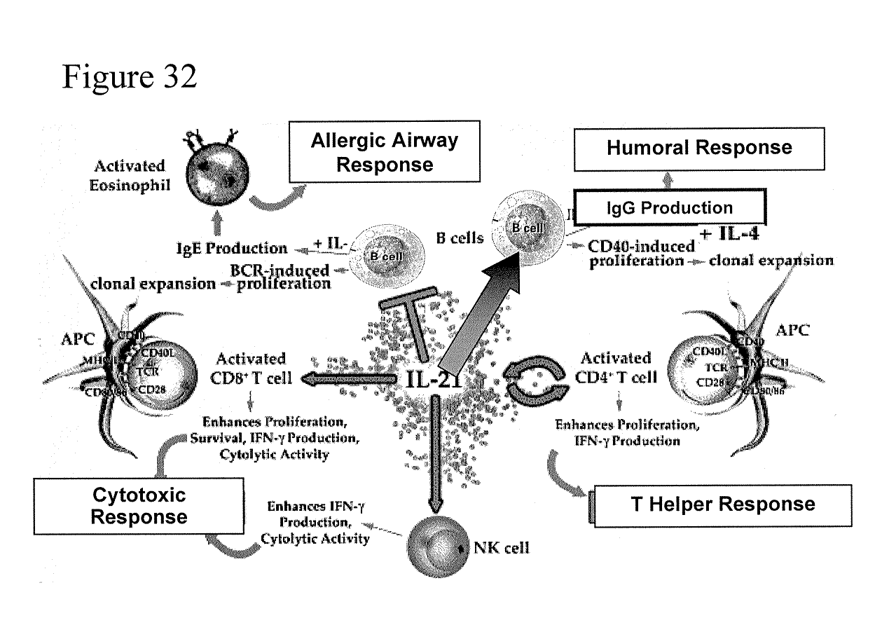

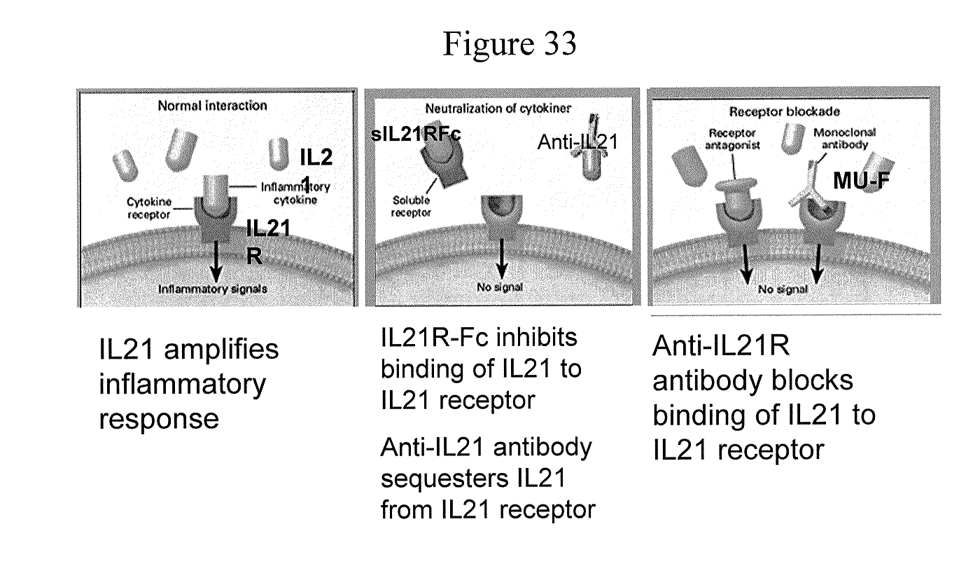

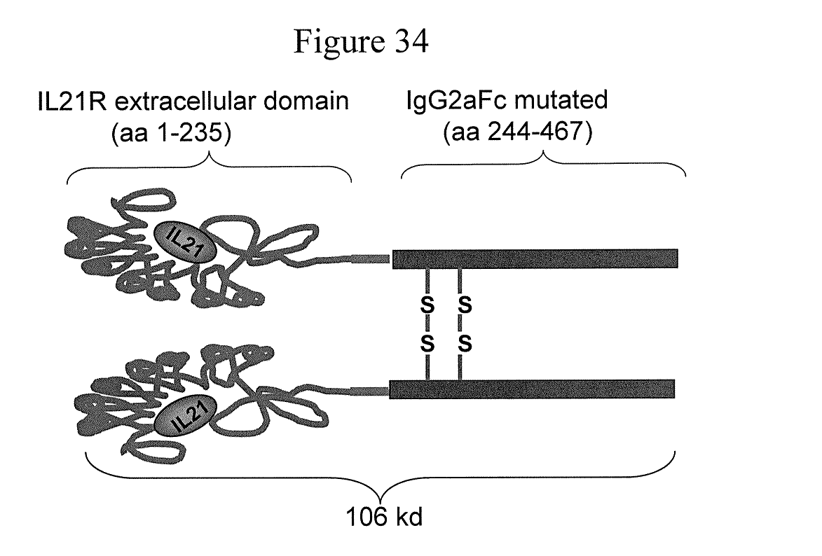

Treatment with MuIL-21RFc also reduced the accumulation of IgG deposits in MRL-Fas^{lpr} mouse kidney. At 15 weeks, mice were sacrificed and frozen kidney sections (5 μm) were stained with goat anti-mouse IgG-FITC. Fluorescence intensity was scored on a scale of 0 to 3. FIG. 31 shows the total fluorescence intensity measured in kidney sections from treated and control mice.

These results show that therapeutic treatment with an IL-21R antagonist can alleviate lupus-like symptoms.

### Example 14

### Animal Model of Lupus and GVHD: Lack of Autoantibody Formation and IgG Deposition in the Kidneys of IL-2R Deficient Mice Engrafted with B6 bm12 Spleen Cells

Experiments were conducted to investigate the response of IL-21R knockout (KO) mice in the chronic graft-versus-host-disease (GVHD) model of systemic lupus erythematosus (SLE) (Chen et al. (1998) J. Immunol. 161:5880-85). This model comprises representative aspects of both SLE and GVHD.

The animals used were: B6.C-H2<bm12>/KhEG (bm12), Jackson Labs (spleen cells); IL-21R-2 KO mice, Charles River Labs (CRL); C57/BL6 wild type (WT) mice, Charles River Labs; and C57/BL6 wild type mice, Taconic (TAC) (Germantown, N.Y.).

Appropriate donor mice were sacrificed on the day of disease induction via CO₂ exposure. Spleens were harvested and mulched. Red blood cells were lysed using 0.16M NHCl: 0.17M TrisCl (9:1) at 1 ml lysis solution per spleen, for a total of 5 minutes with occasional mixing. Cell suspensions were counted using trypan blue and adjusted to a final concentration of 2×10⁸ cells/ml using sterile phosphate buffered saline. 0.5 ml of the appropriate cell suspension was then injected intraperitoneally into the appropriate recipient mouse (as indicated in Table 2, below). The recipient mice were then monitored weekly for urine protein and weight gain/loss. Every two weeks, each mouse was bled via retro-orbital sinus, and the sera were stored for further analysis. ELISA assays were performed on all sera collected at each of the time points (as described in Zouali and Stollar (1986) J. Immunol. Methods 90:105-10) for the detection of autoantibodies against double-stranded DNA.

At 12 weeks post-disease induction, half of the animals from each group were euthanized, and the spleen and both kidneys were collected. The left kidney was preserved (intact) in 10% nonbuffered formalin and stained with H&E. Scoring for staining was performed according to the method of Chen et al., supra. Score parameters included: perivascular lymphocytic infiltration, interstitial lymphocytic infiltration, hypercellularity and basement membrane thickening. The right kidney was cut longitudinally and each half was embedded cut side down in a tissue block cassette. The right kidney was then analyzed using immunohistochemical techniques for the presence of immune deposits, specifically IgG, IgM and C3.

TABLE 2

| | Group | Donor | Recipient | n |
|---|---|---|---|---|
| 1 | IL-21R KO | bm12 | CRL IL-21R KO | 8 |
| 2 | CRL-GVHD (C-GVHD) | bm12 | CRL B6 | 10 |
| 3 | TAC-GVHD (T-GVHD) | bm12 | TAC B6 | 10 |
| 4 | CRL-Control (C-Control) | CRL B6 | CRL B6 | 5 |
| 5 | TAC-Control (T-Control) | TAC B6 | TAC B6 | 5 |

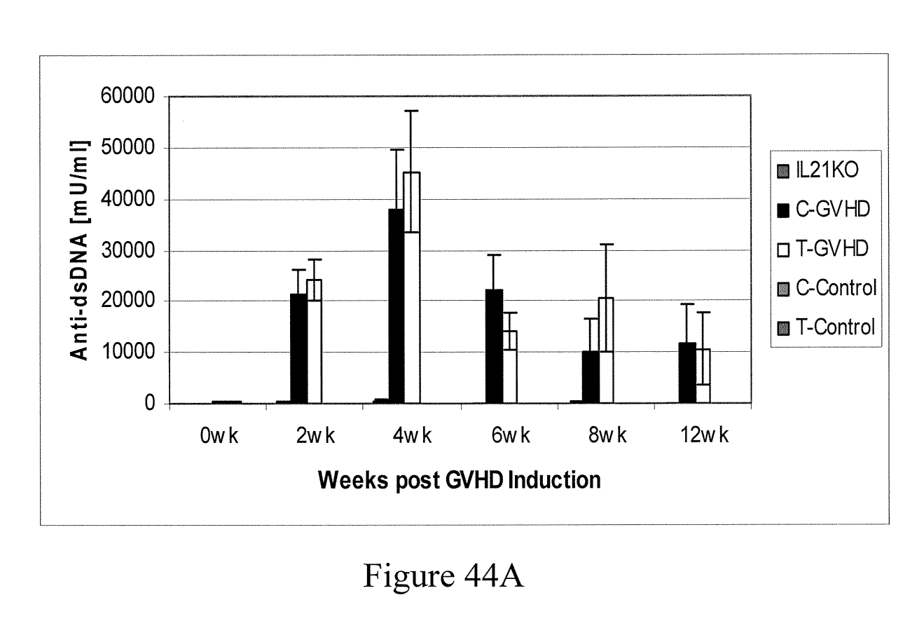

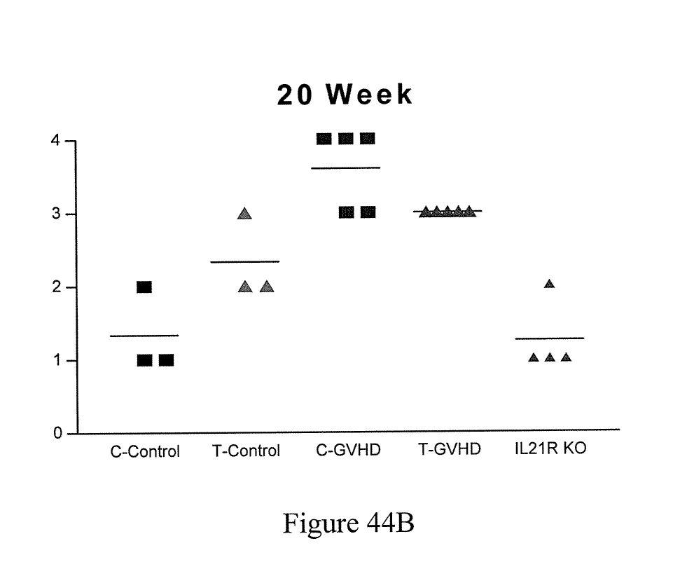

The results from these experiments are shown in FIG. 44. No anti-dsDNA autoantibodies were detected in any of the IL-21R knockout mice at any time point (FIG. 44A). In addition, FIG. 44B shows that at twenty weeks post disease induction, IgG deposition is not observed in the kidneys of IL-21R-deficient mice when compared with GVHD mice. Thus, mice deficient for IL-21R do not generate autoantibodies in the GVHD-SLE model, nor do they form IgG deposits in kidneys. Accordingly, treatment of individuals with IL-21 /IL-21R antagonists may provide an effective therapy for both SLE and GVHD.

The contents of all references, pending patent applications (inclusive of 60/599,086, filed Aug. 5, 2004 and 60/639,176, filed Dec. 23, 2004), published patent applications (inclusive of 2003/0108549, filed Oct. 4, 2002), and published patents cited throughout this application are hereby incorporated by reference.

### Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. A method of treating, ameliorating, or preventing an autoimmune or inflammatory disorder in a mammalian subject, comprising administering to the subject an IL-21/IL-21R antagonist selected from the group consisting of an anti-IL-21R antibody, an anti-IL-21 antibody, an antigen-binding fragment of an anti-IL-21R antibody, an antigen-binding fragment of an anti-IL-2 antibody, and an IL-21R soluble fragment, in an amount sufficient to treat, ameliorate, or prevent the disorder.

2. A method of treating, ameliorating, or preventing a disorder selected from the group consisting of an arthritic disorder, an atopic disorder, a respiratory disorder, a skin inflammatory disorder, an intestinal inflammatory disorder, a fibrotic disorder, systemic lupus erythematosus, transplant/graft rejection, and a disorder associated with transplant/graft rejection, in a mammalian subject, comprising administering to the subject an IL-21/IL-21R antagonist selected from the group consisting of an anti-IL-21R antibody, an anti-IL-21 antibody, an antigen-binding fragment of an anti-IL-21R antibody, an antigen-binding fragment of an anti-IL-21 antibody, and an IL-21R soluble fragment, in an amount sufficient to treat, ameliorate, or prevent the disorder.

3. The method of claim 2, wherein the anti-IL-21R antibody is capable of binding to an IL-21R comprised of an amino acid sequence at least 90% identical to the sequence set forth in SEQ ID NO:2, and wherein the IL-21R is capable of binding IL-21.

4. The method of claim 3, wherein the arthritic disorder is selected from the group consisting of rheumatoid arthritis, juvenile rheumatoid arthritis, osteoarthritis, psoriatic arthritis, and ankylosing spondylitis.

5. The method of claim 4, wherein the arthritic disorder is rheumatoid arthritis.

6. The method of claim 3, wherein the respiratory disorder is asthma or chronic obstructive pulmonary disease.

7. The method of claim 3, wherein the fibrotic disorder is selected from the group consisting of fibrosis of an internal organ, a dermal fibrosing disorder, a fibrotic condition of the eye, systemic sclerosis, polymyositis, dermatomyositis, eosinophilic fasciitis, Raynaud's syndrome, glomerulonephritis and nasal polyposis.

8. The method of claim 3, wherein the intestinal inflammatory disorder is selected from the group consisting of inflammatory bowel disease, ulcerative colitis, and Crohn's disease.

9. The method of claim 3, wherein the skin inflammatory disorder is psoriasis.

10. The method of claim 3, wherein the atopic disorder is selected from the group consisting of allergic asthma, atopic dermatitis, urticaria, eczema, allergic rhinitis, and allergic enterogastritis.

11. The method of claim 10, wherein the atopic disorder is allergic asthma.

12. The method of claim 3, wherein the disorder associated with transplant/graft rejection is graft versus host disease.

13. The method of claim 3, wherein the disorder is transplant/graft rejection.

14. The method of claim 3, wherein the disorder is systemic lupus erythematosus.

15. The method of claim 2, wherein the mammalian subject is a human.

16. The method of claim 2, wherein the IL-21R soluble fragment is comprised of an IL-21R extracellular domain and an Fc immunoglobulin fragment.

17. The method of claim 16, wherein the IL-21R extracellular domain comprises about amino acids 1-235 of SEQ ID NO:2.

18. The method of claim 2, wherein the IL-21R soluble fragment is comprised of an amino acid sequence at least 90% identical to the sequence set forth in SEQ ID NO:29.

19. The method of claim 2, wherein the IL-21/IL-21R antagonist is an anti-IL-21R antibody, or an antigen-binding fragment thereof.

20. The method of claim 2, wherein the IL-21/IL-21R antagonist is an anti-IL-21 antibody, or an antigen-binding fragment thereof.

21. A fusion protein comprised of an extracellular domain of an IL-21R and an Fc immunoglobulin fragment, wherein the IL-21R has an amino acid sequence at least 90% identical to the sequence set forth in SEQ ID NO:2, and wherein the fusion protein is capable of binding IL-21.

22. The fusion protein of claim 21, comprised of an amino acid sequence at least 90% identical to the sequence set forth in SEQ ID NO:29.

23. A vector having a nucleotide sequence encoding the fusion protein of claim 21.

24. A recombinant host cell comprising the vector of claim 23.

25. A method of producing a fusion protein comprising:
(a) culturing the recombinant host cell of claim 24 under conditions such that the fusion protein is expressed; and
(b) recovering the fusion protein.

26. A pharmaceutical composition comprising an IL-21/IL-21R antagonist and a pharmaceutically acceptable carrier.

27. The pharmaceutical composition of claim 26, wherein the IL-21/IL-21R antagonist is selected from the group consisting of an anti-IL-21R antibody, an anti-IL-21 antibody, an antigen-binding fragment of an anti-IL-21R antibody, an antigen-binding fragment of an anti-IL-21 antibody, and an IL-21R soluble fragment.

28. The pharmaceutical composition of claim 27, wherein the IL-21R soluble fragment is comprised of an extracellular domain of an IL-21R and an Fc immunoglobulin fragment.

29. A method of transplanting/grafting an organ, tissue, cell or group of cells to a mammalian subject comprising the steps of:
(a) administering to the subject an antagonist of IL-21/IL-21R selected from the group consisting of an anti-IL-21R antibody, an anti-IL-21 antibody, an antigen-binding fragment of an anti-IL-21R antibody, an antigen-binding fragment of an anti-IL-21 antibody, and an IL-21R soluble fragment, in an amount sufficient to reduce the risk of transplant/graft rejection; and
(b) transplanting/grafting an organ, tissue, cell or group of cells to the subject,
wherein the transplanting/grafting step (b) occurs either before, during, or after the administering step (a).

30. The method of claim 29, wherein the organ, tissue, cell or group of cells transplanted/grafted is selected from the group consisting of heart, kidney, liver, lung, pancreas, bone marrow, cartilage, cornea, neuronal tissue, and cells thereof.

31. A method of treating, preventing or ameliorating transplant/graft rejection in a mammalian transplant/graft recipient comprising:
(a) detecting a symptom of transplant/graft rejection in a transplant/graft recipient; and
(b) administering to the transplant/graft recipient an IL-21 /IL-21R antagonist selected from the group consisting of an anti-IL-21R antibody, an anti-IL-21 antibody, an antigen-binding fragment of an anti-IL-2 IR antibody, an antigen-binding fragment of an anti-IL-21 antibody, and an IL-21R soluble fragment.

32. The method of claim 31, wherein the symptom of transplant/graft rejection is selected from the group consisting of inflammation, decreased organ function, signs of rejection in biopsy, and fibrosis.
